(19) United States
(12) Patent Application Publication
(10) Pub. No.: US 2004/0152106 A1
(43) Pub. Date: Aug. 5, 2004

(54) Gene necessary for striatal function, uses thereof, and compounds for modulating same

(76) Inventors: Harold A. Robertson, Halifax (CA); Eileen M. Denovan-Wright, Halifax (CA)

Correspondence Address: BROMBERG & SUNSTEIN LLP, 125 SUMMER STREET, BOSTON, MA, 02110-1618, US

(21) Appl. No.: 10/659,770

(22) Filed: Sep. 10, 2003

Related U.S. Application Data
(63) Continuation of application No. 09/680,208, filed on Oct. 6, 2000, now abandoned.
(60) Provisional application No. 60/158,043, filed on Oct. 7, 1999.
(60) Provisional application No. 60/217,765, filed on Jul. 12, 2000.

(51) Int. Cl. C12Q 1/68; C12Q 1/42

(52) U.S. Cl. 435/6; 435/21

(57) ABSTRACT

PDE10A, a gene that is normally highly expressed in mammalian striatum and elsewhere, has been found to decrease in expression during the development of CAG repeat disorders such as Huntington's disease. The invention teaches a method for detecting the presence of or the predisposition for a CAG repeat disorder. Compounds which modulate CAG repeat disorders and their uses are taught. Methods for screening for further compounds to modulate CAG repeat disorders are also taught.

### CROSS REFERENCE TO RELATED APPLICATIONS

This patent is a continuation from U.S. patent application Ser. No. 09/680,208, which claims priority from U.S. provisional application No. 60/158,043 filed Oct. 7, 1999, and U.S. provisional application No. 60/217,765 filed Jul. 12, 2000, all of which are hereby incorporated by reference herein in their entirety.

### TECHNICAL FIELD AND BACKGROUND ART

The present invention relates to a polynucleotide, PDE10A, which is down-regulated during the development of CAG repeat disorders, such as Huntington's disease. The present invention also describes compounds that modulate CAG repeat disorders, processes for expressing PDE10A, and its agonists and antagonists, and uses of PDE10A, and its variants, derivatives, agonists and antagonists.

### BACKGROUND OF THE INVENTION

Very few if any effective treatments exist for neurological disorders characterized by progressive cell loss, known as neurodegenerative diseases, as well as those involving acute cell loss, such as stroke and trauma.

Huntington's disease (HD) is an inherited neurological disorder that is transmitted in autosomal dominant fashion. HD results from genetically programmed degeneration of neurons in certain areas of the brain. Huntington's disease is caused by a mutation of the gene IT-15 that codes for the protein huntingtin. The huntingtin gene contains a polymorphic stretch of repeated CAG trinucleotides that encode a polyglutamine tract within huntingtin. If this tract exceeds 35 in number, Huntington's disease results. Huntington's disease is only one of a number of neurological diseases which are characterised by these polyglutamine repeats (Ross, 1997). Schizophrenia, Alzheimer's disease, stroke, trauma, and Parkinson's disease also affect the basal ganglia.

Huntingtin has no sequence similarity to known proteins (Group THDCR, 1993; Sisodia, 1998). The function of the normal or mutated HD form of huntingtin has not been defined by the prior art. It is evident, however, that the expression of the HD form of huntingtin leads to progressive and selective neuronal loss. It has been demonstrated that the GABA- and enkephalin-containing medium spiny projection neurons of the caudate-putamen eventually die as a result of HD (Richfield et al., 1994). Patients with minimal cell loss, however, still present with motor and cognitive symptoms suggesting that neuronal dysfunction, and not simply cell loss, contribute to the symptoms of HD. The motor symptoms of HD include the development of chorea, dystonia, bradykinesia and tremors (Young et al., 1986). Voluntary movements may also be affected such that there may be disturbances in speech (Ludlow et al., 1987) and degradation of fine motor co-ordination (Young et al., 1986). In addition to motor decline, emotional disturbances and cognitive loss are also evident during the progression of HD (Caine et al., 1978).

Despite the fact that huntingtin is ubiquitously expressed, HD specifically affects cells of the basal ganglia, structures deep within the brain that have a number of important functions, including co-ordinating movement. The basal ganglia includes the caudate nucleus, the putamen, the nucleus accumbens and the olfactory tubercule. HD also affects the brain's outer surface, or cortex, which controls thought, perception, and memory. The mechanism by which only a small group of neurons in the striatum and cortex are rendered vulnerable to this ubiquitously expressed mutant protein is not known. There are no effective treatments for Huntington's disease.

Huntington's disease is widely believed to be a gain-of function disorder but neither the normal function nor the gained function of huntingtin is known. Because the function for huntingtin is not known, there is little insight into the disease process. It was believed that huntingtin was related to neuronal intranuclear inclusions (NII). However, recent results have cast doubt on our understanding of the role of the NII in Huntington's disease (Saudou et al., 1998) or in other CAG repeat disorders (Klement et al., 1998; see also commentary by Sisodia, 1998).

The development of a mouse carrying the 5′ end of the human Huntington's disease gene (the promoter and first exon; Mangiarini et al., 1996) was an important step in the development of the tools that will allow us to understand the function (and gain-of-function) associated with huntingtin. R6/2 mice exhibit a rapidly progressing neurological phenotype with onset at about 8 weeks. This phenotype includes a movement disorder characterised by shuddering, resting tremor, epileptic seizures and stereotyped behaviour. These symptoms suggest that the function of the basal ganglia is affected by the expression of the human exon 1 transgene prior to neuronal cell death. By 12 weeks the affected mice have significantly reduced brain weights and they die by about 13 weeks of age. Neuronal intranuclear inclusions (NII) develop at about 4 weeks (Davies et al., 1997). As is observed in human Huntington's disease patient, the R6/2 mice show changes in neuronal receptors (Cha et al., 1998). The present inventors have also demonstrated that changes in the expression of DARPP-32 and cannabinoid receptors change over time in HD mice; such changes have also been observed in human Huntington's disease patients (unpublished results). The loss of the cannabinoid receptor is one of the earliest documented changes that occur prior to neuronal degeneration in human HD patients. The R6/2 model, therefore, mimics the early phases of HD; a point in disease development where intervention would be most appropriate.

Human PDE10 was recently identified by identification of cDNA fragments published on the National Center for Biotechnology Information (NCBI) Expressed Sequence Tags (EST) database (Loughney et al., WO99/42596). While PDE10 was found to share homology with known PDEs, no function could be identified for PDE10.

### SUMMARY OF THE INVENTION

The present invention provides the function and uses of a nucleotide segment, PDE10A, and compounds which inhibit or promote the development of CAG repeat disorders such as Huntington's Disease.

The invention teaches a method for identifying a compound which inhibits or promotes a CAG repeat disorder, comprising the steps of: (a) selecting a control animal having PDE10A and a test animal having PDE10A; (b) treating said test animal using a compound; and (c) determining the relative quantity of RNA corresponding to PDE10A, as between said animals. In an embodiment, the animal is a mammal, preferably a mouse, and preferably a transgenic mouse. In an embodiment, the CAG repeat disorder is Huntington's disease.

The invention also teaches a method for identifying a compound which inhibits or promotes a CAG repeat disorder, comprising the steps of: (a) selecting a host cell containing PDE10A; (b) cloning said host cell and separating said clones into a test group and a control group; (c) treating said test group using a compound; and (c) determining the relative quantity of RNA corresponding to PDE10A, as between said test group and said control group. In an embodiment, the CAG repeat disorder is Huntington's disease.

The invention further teaches a method for detecting the presence of or the predisposition for a CAG repeat disorder, said method comprising determining the level of expression of RNA corresponding to PDE10A in an individual relative to a predetermined control level of expression, wherein a decreased expression of said RNA as compared to said control is indicative of a CAG repeat disorder. Preferably, the expression is measured by in situ hybridization, fluorescent in situ hybridization, polymerase chain reaction, or DNA fingerprinting technique. In an embodiment, the CAG repeat disorder is Huntington's disease.

The invention further teaches compositions for treating a CAG repeat disorder comprising a compound which modulates PDE10 expression and a pharmaceutically acceptable carrier. The compound can be selected from the group consisting of: quinpirole, alloxan, miconazole nitrate, MDL-12330A and tetracyline derivatives such as demeclocycline. The compound may be selected from the group consisting of: (6R,12aR)-2,3,6,7,12,12a-Hexahydro-6-(5-benzofuranyl)-2-methyl-pyrazino[2′,1′:6,1]pyrido[3,4-b]indole-1,4-dione,

(6R,12aR)-2,3,6,7,12,12a-Hexahydro-6-(5-benzofuranyl)-pyrazino[2′1′,:6,1]py rido[3,4-]indole-1,4-dione, (6R,12aR)-2,3,6,7,12,12a-Hexahydro-6-(5-benzofuranyl)-2-isopropyl-pyrazino[2′,1′:6,1]pyrido[3,4-b]indole-1,4-dione, (3S,6R,12aR)-2,3,6,7,12,12a-Hexahydro-6-(5-benzofuranyl)-3-methyl-pyrazino[2′,1′:6,1]pyrido[3,4-b]indole-1,4-dione, and

(3S,6R,12aR)-2,3,6,7,12,12a-Hexahydro-6-(5-benzofuranyl)-2,3-dimethyl-pyraz ino[2′,1′:6,1]pyrido[3,4-b]indole-1,4-dione, or from the group consisting of: KS-505, IC224, SCH 51866, IBMX and Dipyridamole. The disorder can be HD.

The invention also teaches the use of a composition which modulates PDE10 for treating a CAG repeat disorder comprising administering the composition to a subject in need of such treatment, and such use of the composition which modulates PDE10 for treating HD.

### BRIEF DESCRIPTION OF THE DRAWINGS

The foregoing features of the invention will be more readily understood by reference to the following detailed description, taken with reference to the accompanying drawings, in which:

FIG. 1 is a portion of an autoradiogram of the differential display reaction identifying PDE10A in mouse brain mRNA.

FIG. 2 is a northern blot confirming that PDE10A has a lower steady-state level of expression in the striatum of transgenic HD mice.

FIG. 3 is a nucleotide sequence of the differential display cDNA fragment of pPDE10A.

FIG. 4 shows the in situ hybridization of probe 1 to coronal and saggital brain sections of 10 week-old wild-type and HD mice.

FIG. 5 shows the in situ hybridization corresponding to spatial and temporal expression of PDE10A in brain sections of wild-type and HD mice over the period of time that the HD mice develop abnormal movements and postures.

FIG. 6 shows the in situ hybridization corresponding to expression of PDE10A in brain sections of one day old wild-type and HD mice.

FIG. 7 shows the in situ hybridization corresponding to distribution of the mRNA of PDE10A in mouse striatal neurons.

FIG. 8 is the in situ hybridization corresponding to mRNA distribution of the rat homologue of PDE10A in rat brain tissue.

FIG. 9 shows a Southern blot analysis of DNA from wild-type and transgenic HD mice hybridized to the pPDE10A cDNA probe.

FIG. 10 is a nucleotide sequence of cPDE10-1, and corresponds to SEQ ID NO. 1.

FIG. 11 is a restriction map of cPDE10-1.

FIG. 12 is a nucleotide sequence of cPDE10-2, and corresponds to SEQ ID NO. 2.

FIG. 13 is a restriction map of cPDE10-2.

FIG. 14 is a schematic diagram showing the alignment of cPDE10-1 and -2 and the regions that are identical and unique between the two clones.

FIG. 15 is a nucleotide sequence of cPDE10A and RACEs, corresponding to SEQ ID NO. 11.

FIG. 16 is a map of PDE10A coding sequence and restriction sites.

FIG. 17 is a map of PDE10A coding sequence and features.

FIG. 18 is a restriction map of PDE10A.

FIG. 19 is a nucleotide sequence of cPDE10A and corresponds to SEQ ID NO. 12.

### DETAILED DESCRIPTION OF SPECIFIC EMBODIMENTS

The following illustrative explanations are provided to facilitate understanding of certain terms used frequently herein. The explanations are provided as a convenience and are not limitative of the invention.

“Host cell” is a cell which has been transformed or transfected, or is capable of transformation or transfection by an exogenous polynucleotide sequence.

“Identity”, “similarity” or “homologous”, as used in the art, are relationships between two or more polynucleotide sequences, as determined by comparing the sequences. In the art, identity also means the degree of sequence relatedness between polynucleotide sequences, as the case may be, as determined by the match between strings of such sequences. Both identity and similarity can be readily calculated (Lesk, A. M., 1988; Smith, D. W., 1993; Griffin, A. M., and Griffin, H. G., 1994; von Heinje, G., 1987; and Gribskov, M. and Devereux, J., 1991). While there exist a number of methods to measure identity and similarity between two polynucleotide sequences, both terms are well known to skilled artisans (von Heinje, G., 1987; Gribskov, M. and Devereux, 1991; and Carillo, H., and Lipman, D., 1988). Methods commonly employed to determine identity or similarity between sequences include, but are not limited to those disclosed in Carillo, H., and Lipman, D. (1988). Methods to determine identity and similarity are codified in computer programs. Computer program methods to determine identity and similarity between two sequences include, but are not limited to, GCG program package (Devereux, J., et al., 1984), BLASTP, BLASTN, and FASTA (Atschul, S. F. et al., 1990).

“Isolated” means altered “by the hand of man” from its natural state; i.e., that, if it occurs in nature, it has been changed or removed from its original environment, or both. For example, a naturally occurring polynucleotide naturally present in a living organism in its natural state is not “isolated,” but the same polynucleotide separated from coexisting materials of its natural state is “isolated”, as the term is employed herein. As part of or following isolation, such polynucleotides can be joined to other polynucleotides, such as DNA, for mutagenesis, to form fusion proteins, and for propagation or expression in a host, for instance. The isolated polynucleotides, alone or joined to other polynucleotides such as vectors, can be introduced into host cells, in culture or in whole organisms. Introduced into host cells in culture or in whole organisms, such DNA still would be isolated, as the term is used herein, because they would not be in their naturally occurring form or environment. Similarly, the polynucleotides may occur in a composition, such as a media formulations, solutions for introduction of polynucleotides, for example, into cells, compositions or solutions for chemical or enzymatic reactions, for instance, which are not naturally occurring compositions, and, therein remain isolated polynucleotides within the meaning of that term as it is employed herein.

“Plasmids”. Starting plasmids disclosed herein are either commercially available, publicly available, or can be constructed from available plasmids by routine application of well known, published procedures. Many plasmids and other cloning and expression vectors that can be used in accordance with the present invention are well known and readily available to those of skill in the art. Moreover, those of skill readily may construct any number of other plasmids suitable for use in the invention.

“Polynucleotides(s)” of the present invention may be in the form of RNA, such as mRNA, or in the form of DNA, including, for instance, cDNA and genomic DNA obtained by cloning or produced by chemical synthetic techniques or by a combination thereof. The DNA may be double-stranded or single-stranded. Single-stranded polynucleotides may be the coding strand, also known as the sense strand, or it may be the non-coding strand, also referred to as the anti-sense strand. Polynucleotides generally refers to any polyribonucleotide or polydeoxribonucleotide, which may be unmodified RNA or DNA or modified RNA or DNA. Thus, for instance, polynucleotides as used herein refers to, among others, single-and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded, or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term polynucleotide also includes DNA or DNA that contain one or more modified bases. Thus, DNA or DNA with backbones modified for stability or for other reasons are “polynucleotides” as that term is intended herein. Moreover, DNA or DNA comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia. Polynucleotides embraces short polynucleotides often referred to as oligonucleotide(s). It will also be appreciated that RNA made by transcription of this doubled stranded nucleotide sequence, and an antisense strand of a nucleic acid molecule of the invention or an oligonucleotide fragment of the nucleic acid molecule, are contemplated within the scope of the invention. An antisense sequence is constructed by inverting the sequence of a nucleic acid molecule of the invention, relative to its normal presentation for transcription. Preferably, an antisense sequence is constructed by inverting a region preceding the initiation codon or an unconserved region. The antisense sequences may be constructed using chemical synthesis and enzymatic ligation reactions using procedures known in the art.

“Stringent hybridization conditions” are those which are stringent enough to provide specificity, reduce the number of mismatches and yet are sufficiently flexible to allow formation of stable hybrids at an acceptable rate. Such conditions are known to those skilled in the art and are described, for example, in Sambrook, et al, (1989). By way of example only, stringent hybridization with short nucleotides may be carried out at 5-10° below the T_{M} using high concentrations of probe such as 0.01-1.0 pmole/ml. Preferably, the term “stringent conditions” means hybridization will occur only if there is at least 95% and preferably at least 97% identity between the sequences.

“Variant(s)” of polynucleotides are polynucleotides that differ in nucleotide sequence from another, reference polynucleotide. Generally, differences are limited so that the nucleotide sequences of the reference and the variant are closely similar overall and, in many regions, identical. Changes in the nucleotide sequence of the variant may be silent. That is, they may not alter the amino acids encoded by the polynucleotide. Where alterations are limited to silent changes of this type a variant will encode a polypeptide or polynucleotide with the same amino acid sequence as the reference. Changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide or polynucleotide encoded by the reference sequence.

As hereinbefore mentioned, the present inventors have identified and sequenced a DNA sequence encoding PDE10A. The DNA sequence is shown in the Sequence Listing as SEQ ID NO:1, NO:2 and NO:11.

It will be appreciated that the invention includes nucleotide or amino acid sequences which have substantial sequence homology with the nucleotide sequences shown in the Sequence Listing as SEQ ID NO:1, NO:2 and NO:11. The term “sequences having substantial sequence homology” means those nucleotide and amino acid sequences which have slight or inconsequential sequence variations from the sequences disclosed in the Sequence Listing as SEQ ID NO:1, NO:2 and NO:11; i.e. the homologous sequences function in substantially the same manner to produce substantially the same polypeptides as the actual sequences. The variations may be attributable to local mutations or structural modifications. It is expected that a sequence having 85-90% sequence homology with the DNA sequence of the invention will provide a functional PDE10 polypeptide.

As used herein, “PDE10A” comprises a polynucleotide sequence which is down regulated in the course of CAG repeat disorders selected from the group consisting of: (a) a sequence comprising SEQ ID NO:1; (b) a sequence comprising SEQ ID NO:2; (c) a sequence comprising SEQ ID NO:11; (d) a sequence comprising nucleotides 257 to 2596 of SEQ ID NO:11; (e) a sequence which is at least 90% homologous with a sequence of (a), (b), (c) or (d), and; (f) a sequence which hybridizes to (a), (b), (c) or (d) under stringent conditions. In an embodiment, the isolated polynucleotide segment is cDNA. The invention also teaches an isolated polynucleotide segment, which retains substantially the same biological function or activity as the polynucleotide encoded by the polynucleotide sequence.

Further embodiments of the invention are polynucleotides that are at least 70% identical over their entire length to a polynucleotide encoding PDE10 polypeptide or polynucleotide, and polynucleotides which are complementary to such polynucleotides. Other embodiments are polynucleotides that comprise a region that is at least 80% identical over their entire length to a polynucleotide encoding PDE10 of SEQ ID NO.11 and polynucleotides complementary thereto. This includes polynucleotides at least 90% identical over their entire length to the same, and among these embodiments are polynucleotides with at least 95%. Furthermore, those with at least 97% are highly preferred among those with at least 95%, and among these those with at least 98% and at least 99% are particularly highly preferred, with at least 99% being the more preferred.

The polynucleotides of the present invention may be employed as research reagents and materials for discovery of treatments of and diagnostics for disease, particularly human disease, as further discussed herein.

Analysis of the complete nucleotide and amino acid sequences of the protein of the invention using the procedures of Sambrook et al., supra, have been used to determine the expressed region, initiation codon and untranslated sequences of the PDE10A gene. The transcription regulatory sequences of the gene are determined by analyzing fragments of the DNA for their ability to express a reporter gene such as the bacterial gene lacZ.

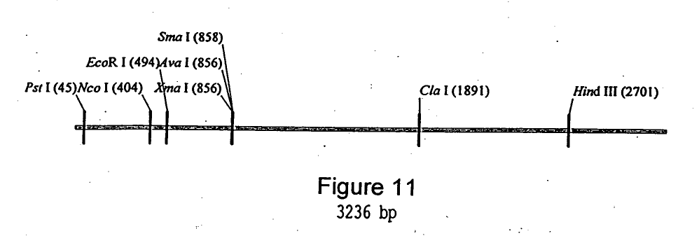

The nucleic acid molecules of the invention allow those skilled in the art to construct nucleotide probes for use in the detection of nucleotide sequences in biological materials. As shown in FIGS. 11, 13, 15 and 16, a number of unique restriction sequences for restriction enzymes are incorporated in the nucleic acid molecule identified in the Sequence Listing as SEQ ID NO:1, NO:2 and NO:11, and these provide access to nucleotide sequences which code for polypeptides unique to the PDE10A polypeptide of the invention. Nucleotide sequences unique to PDE10A or isoforms thereof, can also be constructed by chemical synthesis and enzymatic ligation reactions carried out by procedures known in the art.

A nucleotide probe may be labeled with a detectable marker such as a radioactive label which provides for an adequate signal and has sufficient half-life such as 32p, 3H, 14C or the like. Other detectable markers which may be used include antigens that are recognized by a specific labeled antibody, fluorescent compounds, enzymes, antibodies specific for a labeled antigen, and chemiluminescent compounds. An appropriate label may be selected having regard to the rate of hybridization and binding of the probe to the nucleotide to be detected and the amount of nucleotide available for hybridization. The nucleotide probes may be used to detect genes related to or analogous to PDE10A of the invention.

Accordingly, the present invention also provides a method of detecting the presence of nucleic acid molecules encoding a polypeptide related to or analogous to PDE10A in a sample comprising contacting the sample under hybridization conditions with one or more of the nucleotide probes of the invention labeled with a detectable marker, and determining the degree of hybridization between the nucleic acid molecule in the sample and the nucleotide probes.

Hybridization conditions which may be used in the method of the invention are known in the art and are described for example in Sambrook J, et al., supra. The hybridization product may be assayed using techniques known in the art. The nucleotide probe may be labeled with a detectable marker as described herein and the hybridization product may be assayed by detecting the detectable marker or the detectable change produced by the detectable marker.

The nucleic acid molecule of the invention also permits the identification and isolation, or synthesis of nucleotide sequences which may be used as primers to amplify a polynucleotide molecule of the invention, for example in polymerase chain reaction (PCR). The length and bases of the primers for use in the PCR are selected so that they will hybridize to different strands of the desired sequence and at relative positions along the sequence such that an extension product synthesized from one primer when it is separated from its template can serve as a template for extension of the other primer into a nucleic acid of defined length.

Primers which may be used in the invention are oligonucleotides i.e. molecules containing two or more deoxyribonucleotides of the nucleic acid molecule of the invention which occur naturally as in a purified restriction endonuclease digest or are produced synthetically using techniques known in the art such as, for example, phosphotriester and phosphodiester methods (See Good et al, 1977) or automated techniques (see, for example, Conolly, B. A., 1987). The primers are capable of acting as a point of initiation of synthesis when placed under conditions which permit the synthesis of a primer extension product which is complementary to the DNA sequence of the invention e.g. in the presence of nucleotide substrates, an agent for polymerization such as DNA polymerase and at suitable temperature and pH. Preferably, the primers are sequences that do not form secondary structures by base pairing with other copies of the primer or sequences that form a hair pin configuration. The primer may be single or double-stranded. When the primer is double-stranded it may be treated to separate its strands before using it to prepare amplification products. The primer preferably contains between about 7 and 25 nucleotides.

The primers may be labeled with detectable markers which allow for detection of the amplified products. Suitable detectable markers are radioactive markers such as P-32, S-35, I-125, and H-3, luminescent markers such as chemiluminescent markers, preferably luminol, and fluorescent markers, preferably dansyl chloride, fluorcein-5-isothiocyanate, and 4-fluor-7-nitrobenz-2-axa-1,3 diazole, enzyme markers such as horseradish peroxidase, alkaline phosphatase, .beta.-galactosidase, acetylcholinesterase, or biotin.

It will be appreciated that the primers may contain non-complementary sequences provided that a sufficient amount of the primer contains a sequence which is complementary to a nucleic acid molecule of the invention or oligonucleotide sequence thereof, which is to be amplified. Restriction site linkers may also be incorporated into the primers allowing for digestion of the amplified products with the appropriate restriction enzymes facilitating cloning and sequencing of the amplified product.

Thus, a method of determining the presence of a nucleic acid molecule having a sequence encoding PDE10A or a predetermined oligonucleotide fragment thereof in a sample, is provided comprising treating the sample with primers which are capable of amplifying the nucleic acid molecule or the predetermined oligonucleotide fragment thereof in a polymerase chain reaction to form amplified sequences, under conditions which permit the formation of amplified sequences and, assaying for amplified sequences.

The polymerase chain reaction refers to a process for amplifying a target nucleic acid sequence as generally described in Innis et al, Academic Press, 1989, in Mullis el al., U.S. Pat. No. 4,863,195 and Mullis, U.S. Pat. No. 4,683,202 which are incorporated herein by reference. Conditions for amplifying a nucleic acid template are described in M. A. Innis and D. H. Gelfand, 1989, which is also incorporated herein by reference.

The amplified products can be isolated and distinguished based on their respective sizes using techniques known in the art. For example, after amplification, the DNA sample can be separated on an agarose gel and visualized, after staining with ethidium bromide, under ultra violet (UV) light. DNA may be amplified to a desired level and a further extension reaction may be performed to incorporate nucleotide derivatives having detectable markers such as radioactive labeled or biotin labeled nucleoside triphosphates. The primers may also be labeled with detectable markers. The detectable markers may be analyzed by restriction and electrophoretic separation or other techniques known in the art.

The conditions which may be employed in the methods of the invention using PCR are those which permit hybridization and amplification reactions to proceed in the presence of DNA in a sample and appropriate complementary hybridization primers. Conditions suitable for the polymerase chain reaction are generally known in the art. For example, see M. A. Innis and D. H. Gelfand, 1989, which is incorporated herein by reference. Preferably, the PCR utilizes polymerase obtained from the thermophilic bacterium Thermus aquatics (Taq polymerase, GeneAmp Kit, Perkin Elmer Cetus) or other thermostable polymerase may be used to amplify DNA template strands.

It will be appreciated that other techniques such as the Ligase Chain Reaction (LCR) and Nucleic-Acid Sequence Based Amplification (NASBA) may be used to amplify a nucleic acid molecule of the invention. In LCR, two primers which hybridize adjacent to each other on the target strand are ligated in the presence of the target strand to produce a complementary strand (Barney, 1991 and European Published Application No. 0320308, published Jun. 14, 1989). NASBA is a continuous amplification method using two primers, one incorporating a promoter sequence recognized by an RNA polymerase and the second derived from the complementary sequence of the target sequence to the first primer (U.S. Pat. No. 5,130,238 to Malek).

The present invention also teaches vectors which comprise a polynucleotide or polynucleotides of the present invention, host cells which are genetically engineered with vectors of the invention and the production of polynucleotides of the invention by recombinant techniques.

In accordance with this aspect of the invention the vector may be, for example, a plasmid vector, a single or double-stranded phage vector, a single or double-stranded RNA or DNA viral vector. In certain embodiments in this regard, the vectors provide for specific expression. Such specific expression may be inducible expression or expression only in certain types of cells or both inducible and cell-specific. Particular among inducible vectors are vectors that can be induced for expression by environmental factors that are easy to manipulate, such as temperature and nutrient additives. A variety of vectors suitable to this aspect of the invention, including constitutive and inducible expression vectors for use in prokaryotic and eukaryotic hosts, are well known and employed routinely by those of skill in the art. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids, all may be used for expression in accordance with this aspect of the present invention.

The following vectors, which are commercially available, are provided by way of example. Among vectors for use in bacteria are pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia, and pBR322 (ATCC 37017). Among eukaryotic vectors are pWLNEO, pSV2CAT, pOG44, pXT1 and pSG available from Stratagene; and pSVK3, pBPV, pMSG and pSVL available from Pharmacia. These vectors are listed solely by way of illustration of the many commercially available and well known vectors that are available to those of skill in the art for use in accordance with this aspect of the present invention. It will be appreciated that any other plasmid or vector suitable for, for example, introduction, maintenance, propagation or expression of a polynucleotide or polypeptide of the invention in a host may be used in this aspect of the invention. Generally, any vector suitable to maintain, propagate or express polynucleotides to express a polypeptide or polynucleotide in a host may be used for expression in this regard.

The appropriate DNA sequence may be inserted into the vector by any of a variety of well-known and routine techniques. In general, expression constructs will contain sites for transcription initiation and termination, and, in the transcribed region, a ribosome binding site for translation. The coding portion of the mature transcripts expressed by the constructs will include a translation initiating AUG at the beginning and a termination codon appropriately positioned at the end of the polynucleotide to be translated.

The DNA sequence in the expression vector is operatively linked to appropriate expression control sequence(s), including, for instance, a promoter to direct mRNA transcription. Promoter regions can be selected from any desired gene using vectors that contain a reporter transcription unit lacking a promoter region, such as a chloramphenicol acetyl transferase (“CAT”) transcription unit, downstream of restriction site or sites for introducing a candidate promoter fragment; i.e., a fragment that may contain a promoter. As is well known, introduction into the vector of a promoter-containing fragment at the restriction site upstream of the cat gene engenders production of CAT activity, which can be detected by standard CAT assays. Vectors suitable to this end are well known and readily available, such as pKK232-8 and pCM7. Promoters for expression of polynucleotides of the present invention include not only well known and readily available promoters, but also promoters that readily may be obtained by the foregoing technique, using a reporter gene. Among known prokaryotic promoters suitable for expression of polynucleotides and polypeptides in accordance with the present invention are the E. coli lacI and lacZ and promoters, the T3 and T7 promoters, the gpt promoter, the lambda PR, PL promoters and the trp promoter. Among known eukaryotic promoters suitable in this regard are the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter.

Vectors for propagation and expression generally will include selectable markers and amplification regions, such as, for example, those set forth in Sambrook et al., supra.

As hereinbefore mentioned, the present invention also teaches host cells which are genetically engineered with vectors of the invention.

Polynucleotide constructs in host cells can be used in a conventional manner to produce the gene product encoded by the recombinant sequence. The PDE10A polynucleotide or polypeptide products or isoforms or parts thereof, may be obtained by expression in a suitable host cell using techniques known in the art. Suitable host cells include prokaryotic or eukaryotic organisms or cell lines, for example bacterial, mammalian, yeast, or other fungi, viral, plant or insect cells. Methods for transforming or transfecting cells to express foreign DNA are well known in the art (See for example, Itakura et al., U.S. Pat. No. 4,704,362; Hinnen et al., 1978; Murray et al., U.S. Pat. No. 4,801,542; Upshall et al., U.S. Pat. No. 4,935,349; Hagen et al., U.S. Pat. No. 4,784,950; Axel et al., U.S. Pat. No. 4,399,216; Goeddal et al., U.S. Pat. No. 4,766,075; and Sambrook et al, 1989, all of which are incorporated herein by reference). Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells.

Host cells can be genetically engineered to incorporate polynucleotides and express polynucleotides of the present invention. Introduction of polynucleotides into the host cell can be affected by calcium phosphate transfection, DEAE-dextran mediated transfection, transvection, microinjection, cationic lipid-mediated transfection, electroporation, transduction, scrape loading, ballistic introduction, infection or other methods. Such methods are described in many standard laboratory manuals, such as Davis et al. (1986) and Sambrook et al. (1989).

As hereinbefore mentioned, the present invention also teaches the production of polynucleotides of the invention by recombinant techniques.

The PDE10 polynucleotides encode a polypeptide which is the mature protein plus additional amino or carboxyl-terminal amino acids, or amino acids interior to the mature polypeptide (when the mature form has more than one polypeptide chain, for instance). Such sequences may play a role in processing of a protein from precursor to a mature form, may allow protein transport, may lengthen or shorten protein half-life or may facilitate manipulation of a protein for assay or production, among other things. As generally is the case in vivo, the additional amino acids may be processed away from the mature protein by cellular enzymes.

A precursor protein, having the mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

In sum, a polynucleotide of the present invention may encode a mature protein, a mature protein plus a leader sequence (which may be referred to as a preprotein), a precursor of a mature protein having one or more prosequences which are not the leader sequences of a preprotein, or a preproprotein, which is a precursor to a proprotein, having a leader sequence and one or more prosequences, which generally are removed during processing steps that produce active and mature forms of the polypeptide.

The polypeptides of the invention may be prepared by culturing the host/vector systems described above, in order to express the recombinant polypeptides. Recombinantly produced PDE10A based protein or parts thereof, may be further purified using techniques known in the art such as commercially available protein concentration systems, by salting out the protein followed by dialysis, by affinity chromatography, or using anion or cation exchange resins.

Mature proteins can be expressed in mammalian cells, yeast, bacteria, or other cells under the control of appropriate promoters. Cell-free translation systems can also be employed to produce such proteins using DNA derived from the DNA constructs of the present invention. Appropriate cloning and expression vectors for use with prokaryotic and eukaryotic hosts are described by Sambrook et al., supra.

Polynucleotides of the invention, encoding the heterologous structural sequence of a polynucleotide or polypeptide of the invention generally will be inserted into a vector using standard techniques so that it is operably linked to the promoter for expression. The polynucleotide will be positioned so that the transcription start site is located appropriately 5′ to a ribosome binding site. The ribosome binding site will be 5′ to the AUG that initiates translation of the polynucleotide or polypeptide to be expressed. Generally, there will be no other open reading frames that begin with an initiation codon, usually AUG, and lie between the ribosome binding site and the initiation codon. Also, generally, there will be a translation stop codon at the end of the expressed polynucleotide and there will be a polyadenylation signal in constructs for use in eukaryotic hosts. Transcription termination signal appropriately disposed at the 3′ end of the transcribed region may also be included in the polynucleotide construct.

For secretion of the translated protein into the lumen of the endoplasmic reticulum, into the periplasmic space or into the extracellular environment, appropriate secretion signals may be incorporated into the expressed polynucleotide or polypeptide. These signals may be endogenous to the polynucleotide or they may be heterologous signals. Microbial cells employed in expression of proteins can be disrupted by any convenient method, including freeze-thaw cycling, sonication, mechanical disruption, or use of cell lysing agents, such methods are well know to those skilled in the art. PDE10A polynucleotide or polypeptide can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polynucleotide is denatured during isolation and or purification.

In an embodiment, a nucleic acid molecule of the invention may be cloned into a glutathione S-transferase (GST) gene fusion system for example the pGEX-1 T, pGEX-2T and pGEX-3X of Pharmacia. The fused gene may contain a strong lac promoter, inducible to a high level of expression by IPTG, as a regulatory element. Thrombin or factor Xa cleavage sites may be present which allow proteolytic cleavage of the desired polypeptide from the fusion product. The glutathione S-transferase-PDE10A fusion protein may be easily purified using a glutathione sepharose 4B column, for example from Pharmacia. The 26 kd glutathione S-transferase polypeptide can be cleaved by thrombin (pGEX-1 or pGEX-2T) or factor Xa (pGEX-3X) and resolved from the using the polypeptide using the same affinity column. Additional chromatographic steps can be included if necessary, for example Sephadex or DEAE cellulose. The two enzymes may be monitored by protein and enzymatic assays and purity may be confirmed using SDS-PAGE.

The PDE10A protein or parts thereof may also be prepared by chemical synthesis using techniques well known in the chemistry of proteins such as solid phase synthesis (Merrifield, 1964) or synthesis in homogenous solution (Houbenweyl, 1987).

Within the context of the present invention, PDE10A polypeptide includes various structural forms of the primary protein which retain biological activity. For example, PDE10A polypeptide may be in the form of acidic or basic salts or in neutral form. In addition, individual amino acid residues may be modified by oxidation or reduction. Furthermore, various substitutions, deletions or additions may be made to the amino acid or nucleic acid sequences, the net effect being that biological activity of PDE10A is retained. Due to code degeneracy, for example, there may be considerable variation in nucleotide sequences encoding the same amino acid.

The polypeptide may be expressed in a modified form, such as a fusion protein, and may include not only secretion signals but also additional heterologous functional regions. Thus, for instance, a region of additional amino acids, particularly charged amino acids, may be added to the C- or N-terminus of the polypeptide to improve stability and persistence in the host cell, during purification or during subsequent handling and storage. Also, fusion proteins may be added to the polynucleotide or polypeptide to facilitate purification. Such regions may be removed prior to final preparation of the polynucleotide or polypeptide. The addition of peptide moieties to polynucleotide or polypeptides to engender secretion or excretion, to improve stability or to facilitate purification, among others, are familiar and routine techniques in the art. In drug discovery, for example, proteins have been fused with antibody Fc portions for the purpose of high-throughput screening assays to identify antagonists (see Bennett et al., 1995, and Johanson et al., 1995).

Detecting Presence of or Predisposition for CAG Repeat Disorders

This invention is also related to the use of the PDE10A polynucleotides to detect complementary polynucleotides as a diagnostic reagent. Detection of the level of expression of PDE10A in a eukaryote, particularly a mammal, and especially a human, will provide a method for diagnosis of a disease. Eukaryotes (herein also “individual(s)”), particularly mammals, and especially humans, exhibiting decreased levels of PDE10A may be detected by a variety of techniques. Nucleic acids for diagnosis may be obtained from an infected individual's cells and tissues, such as the striatum, nucleus accumbens and olfactory tubercule. RNA may be used directly for detection or may be amplified enzymatically by using PCR (Saiki et al., 1986) prior to analysis. As an example, PCR primers complementary to the nucleic acid encoding PDE10A can be used to identify and analyze PDE10A presence and/or expression. Using PCR, characterization of the level of PDE10A present in the individual may be made by comparative analysis.

The invention thus provides a process for detecting disease by using methods known in the art and methods described herein to detect decreased expression of PDE10 polynucleotide. For example, decreased expression of PDE10 polynucleotide can be measured using any on of the methods well known in the art for the quantification of polynucleotides, such as, for example, PCR, RT-PCR, DNAse protection, northern blotting and other hybridization methods. Thus, the present invention provides a method for detecting triplet-repeat disorders, and a method for detecting a genetic pre-disposition for triplet-repeat disorders and other disorders of the basal ganglia including schizophrenia, stroke, trauma, Parkinson's disease and Alzheimer's disease (AD). More generally, the present invention provides a method for detecting a genetic pre-disposition for neurological disorders characterized by progressive cell loss.

Drug Screening Assays

The invention also provides a method of screening compounds to identify those which enhance (agonist) or block (antagonist) the action of PDE10 polypeptides or polynucleotides, such as its interaction with PDE10-binding molecules. The identification of mutations in specific genes in inherited neurodegenerative disorders, combined with advances in the field of transgenic methods, provides those of skill in the art with the information necessary to further study human diseases. This is extraordinarily useful in modeling familial forms of triplet-repeat disorders and other disorders of the basal ganglia including schizophrenia, stroke, trauma, Parkinson's disease and Alzheimer's disease (AD). More generally, the present invention is useful for modeling neurological disorders characterized by progressive cell loss, as well as those involving acute cell loss, such as stroke and trauma.

For example, to screen for agonists or antagonists, a synthetic reaction mix, a cellular compartment, such as a membrane, cell envelope or cell wall, or a preparation of any thereof, may be prepared from a cell that expresses a molecule that binds PDE10. The preparation is incubated with labeled PDE10 in the absence or the presence of a candidate molecule which may be a PDE10 agonist or antagonist. The ability of the candidate molecule to bind the binding molecule is reflected in decreased binding of the labeled ligand.

PDE10-like effects of potential agonists and antagonists may by measured, for instance, by determining activity of a reporter system following interaction of the candidate molecule with a cell or appropriate cell preparation, and comparing the effect with that of PDE10 or molecules that elicit the same effects as PDE10. Reporter systems that may be useful in this regard include, but are not limited to, colorimetric labeled substrate converted into product, a reporter gene that is responsive to changes in PDE10 activity, and binding assays known in the art.

Another example of an assay for PDE10 antagonists is a competitive assay that combines PDE10 and a potential antagonist with membrane-bound PDE10-binding molecules, recombinant PDE10 binding molecules, natural substrates or ligands, or substrate or ligand mimetics, under appropriate conditions for a competitive inhibition assay. PDE10 can be labeled, such as by radioactivity or a colorimetric compound, such that the number of PDE10 molecules bound to a binding molecule or converted to product can be determined accurately to assess the effectiveness of the potential antagonist.

Potential antagonists include small organic molecules, peptides, polypeptides and antibodies that bind to a polynucleotide or polypeptide of the invention and thereby inhibit or extinguish its activity. Potential antagonists also may be small organic molecules, a peptide, a polypeptide such as a closely related protein or antibody that binds the same sites on a binding molecule, such as a binding molecule, without inducing PDE10-induced activities, thereby preventing the action of PDE10 by excluding PDE10 from binding.

Potential antagonists include a small molecule which binds to and occupies the binding site of the polypeptide thereby preventing binding to cellular binding molecules, such that normal biological activity is prevented. Examples of small molecules include but are not limited to small organic molecules, peptides or peptide-like molecules. Other potential antagonists include antisense molecules (see Okano, 1988, for a description of these molecules). Potential antagonists include compounds related to and derivatives of PDE10.

Developing modulators of the biological activities of specific PDEs requires differentiating PDE isozymes present in a particular assay preparation. The classical enzymological approach of isolating PDEs from natural tissue sources and studying each new isozyme may be used. Another approach has been to identify assay conditions which might favor the contribution of one isozyme and minimize the contribution of others in a preparation. Still another approach has been the separation of PDEs by immunological means. Each of the foregoing approaches for differentiating PDE isozymes is time consuming. As a result many attempts to develop selective PDE modulators have been performed with preparations containing more than one isozyme. Moreover, PDE preparations from natural tissue sources are susceptible to limited proteolysis and may contain mixtures of active proteolytic products that have different kinetic, regulatory and physiological properties than the full length PDEs.

Recombinant PDE10 polypeptide products of the invention greatly facilitate the development of new and specific PDE10 modulators. The need for purification of an isozyme can be avoided by expressing it recombinantly in a host cell that lacks endogenous phosphodiesterase activity (e.g., yeast strain YKS45 deposited as ATCC 74225). Once a compound that modulates the activity of the PDE10 is discovered, its selectivity can be evaluated by comparing its activity on the PDE10 to its activity on other PDE isozymes. Thus, the combination of the recombinant PDE10 products of the invention with other recombinant PDE products in a series of independent assays provides a system for developing selective modulators of PDE10. Selective modulators may include, for example, antibodies and other proteins or peptides which specifically bind to the PDE10 or PDE10 nucleic acid, oligonucleotides which specifically bind to the PDE10 (see Patent Cooperation Treaty International Publication No. WO93/05182 published Mar. 18, 1993 which describes methods for selecting oligonucleotides which selectively bind to target biomolecules) or PDE10 nucleic acid (e.g., antisense oligonucleotides) and other non-peptide natural or synthetic compounds which specifically bind to the PDE10 or PDE10 nucleic acid. Mutant forms of the PDE10 which alter the enzymatic activity of the PDE10 or its localization in a cell are also contemplated. Crystallization of recombinant PDE10 alone and bound to a modulator, analysis of atomic structure by X-ray crystallography, and computer modelling of those structures are methods useful for designing and optimizing non-peptide selective modulators. See, for example, Erickson et al., Ann. Rep. Med. Chem., 27: 271-289 (1992) for a general review of structure-based drug design.

Targets for the development of selective modulators include, for example: (1) the regions of the PDE10 which contact other proteins and/or localize the PDE10 within a cell, (2) the regions of the PDE10 which bind substrate, (3) the allosteric cGMP-binding site(s) of PDE10, (4) the metal-binding regions of the PDE10, (5) the phosphorylation site(s) of PDE10 and (6) the regions of the PDE10 which are involved in dimerization of PDE10 subunits.

Thus, the present invention provides a method for screening and selecting compounds which promote triplet-repeat disorders, and a method for screening and selecting compounds which treat or inhibit triplet-repeat disorders, as well as schizophrenia, stroke, trauma, Parkinson's disease and Alzheimer's disease. More generally, the present invention provides a method for screening and selecting compounds which promote or inhibit neurological disorders characterized by progressive cell loss, as well as those involving acute cell loss, such as stroke and trauma.

The selected antagonists and agonists may be administered, for instance, to inhibit progressive and acute neurological disorders, such as Huntington's disease, Parkinson's disease, schizophrenia, Alzheimer's disease (AD), stroke or trauma.

Antagonists and agonists and other compounds of the present invention may be employed alone or in conjunction with other compounds, such as therapeutic compounds. The pharmaceutical compositions may be administered in any effective, convenient manner including, for instance, administration by direct microinjection into the affected area, or by intravenous or other routes. These compositions of the present invention may be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of antagonists or agonists of the invention and a pharmaceutically acceptable carrier or excipient. Such carriers may include, but are not limited to, saline, buffered saline, dextrose, water, glycerol, ethanol and combinations thereof. The formulation is prepared to suit the mode of administration.

Inhibition of PDE10A will be highly detrimental to striatal brain function. The progressive decline in PDE10A mRNA levels in HD may lead to dysregulation of cAMP levels and neuronal dysfunction. Up-regulation of PDE10A will be effective in combating such neuronal dysfunction.

Gene Therapy

A variety of gene therapy approaches may be used in accordance with the invention to modulate expression of the PDE10A gene in vivo. For example, antisense DNA molecules may be engineered and used to block translation of PDE10A mRNA in vivo. Alternatively, ribozyme molecules may be designed to cleave and destroy the PDE10A mRNAs in vivo. In another alternative, oligonucleotides designed to hybridize to the 5′ region of the PDE10A gene (including the region upstream of the coding sequence) and form triple helix structures may be used to block or reduce transcription of the PDE10A gene. In yet another alternative, nucleic acid encoding the full length wild-type PDE10A message may be introduced in vivo into cells which otherwise would be unable to produce the wild-type PDE10A gene product in sufficient quantities or at all.

In a preferred embodiment, the antisense, ribozyme and triple helix nucleotides are designed to inhibit the translation or transcription of PDE10A. To accomplish this, the oligonucleotides used should be designed on the basis of relevant sequences unique to PDE10A.

For example, and not by way of limitation, the oligonucleotides should not fall within those region where the nucleotide sequence of PDE10A is most homologous to that of other PDEs, such as PDE2 PDE5 and PDE6, herein referred to as “unique regions”.

In the case of antisense molecules, it is preferred that the sequence be chosen from the unique regions. It is also preferred that the sequence be at least 18 nucleotides in length in order to achieve sufficiently strong annealing to the target mRNA sequence to prevent translation of the sequence. Izant and Weintraub, 1984, Cell, 36:1007-1015; Rosenberg et al., 1985, Nature, 313:703-706.

In the case of the “hammerhead” type of ribozymes, it is also preferred that the target sequences of the ribozymes be chosen from the unique regions. Ribozymes are RNA molecules which possess highly specific endoribonuclease activity. Hammerhead ribozymes comprise a hybridizing region which is complementary in nucleotide sequence to at least part of the target RNA, and a catalytic region which is adapted to cleave the target RNA. The hybridizing region contains nine (9) or more nucleotides. Therefore, the hammerhead ribozymes of the present invention have a hybridizing region which is complementary to the sequences listed above and is at least nine nucleotides in length. The construction and production of such ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech endoribonucleases have an eight base pair active site which hybridizes to a target RNA sequence whereafter cleavage of the target RNA takes place. The invention encompasses those Cech-type ribozymes which target eight base-pair active site sequences that are present in PDE10A but not other PDEs.

The foregoing compounds can be administered by a variety of methods which are known in the art including, but not limited to the use of liposomes as a delivery vehicle. Naked DNA or RNA molecules may also be used where they are in a form which is resistant to degradation such as by modification of the ends, by the formation of circular molecules, or by the use of alternate bonds including phosphothionate and thiophosphoryl modified bonds. In addition, the delivery of nucleic acid may be by facilitated transport where the nucleic acid molecules are conjugated to poly-lysine or transferrin. Nucleic acid may also be transported into cells by any of the various viral carriers, including but not limited to, retrovirus, vaccinia, AAV, and adenovirus.

Alternatively, a recombinant nucleic acid molecule which encodes, or is, such antisense, ribozyme, triple helix, or PDE10A molecule can be constructed. This nucleic acid molecule may be either RNA or DNA. If the nucleic acid encodes an RNA, it is preferred that the sequence be operatively attached to a regulatory element so that sufficient copies of the desired RNA product are produced. The regulatory element may permit either constitutive or regulated transcription of the sequence. In vivo, that is, within the cells or cells of an organism, a transfer vector such as a bacterial plasmid or viral RNA or DNA, encoding one or more of the RNAs, may be transfected into cells e.g. (Llewellyn et al., 1987, J. Mol. Biol., 195:115-123; Hanahan et al. 1983, J. Mol. Biol., 166:557-580). Once inside the cell, the transfer vector may replicate, and be transcribed by cellular polymerases to produce the RNA or it may be integrated into the genome of the host cell. Alternatively, a transfer vector containing sequences encoding one or more of the RNAs may be transfected into cells or introduced into cells by way of micromanipulation techniques such as microinjection, such that the transfer vector or a part thereof becomes integrated into the genome of the host cell.

Composition, Formulation, and Administration of Pharmaceutical Compositions

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. Naturally, the proportions of a co-solvent system may be varied considerably without destroying its solubility and toxicity characteristics. Furthermore, the identity of the co-solvent components may be varied.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

The pharmaceutical compositions generally are administered in an amount effective for treatment or prophylaxis of a specific indication or indications. It is appreciated that optimum dosage will be determined by standard methods for each treatment modality and indication, taking into account the indication, its severity, route of administration, complicating conditions and the like. In therapy or as a prophylactic, the active agent may be administered to an individual as an injectable composition, for example as a sterile aqueous dispersion, preferably isotonic. A therapeutically effective dose further refers to that amount of the compound sufficient to result in amelioration of symptoms associated with such disorders. Techniques for formulation and administration of the compounds of the instant application may be found in “Remington's Pharmaceutical Sciences,” Mack Publishing Co., Easton, Pa., latest edition. For administration to mammals, and particularly humans, it is expected that the daily dosage level of the active agent will be from 0.001 mg/kg to 10 mg/kg, typically around 0.01 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

The invention further provides diagnostic and pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

### EXAMPLES

The present invention is further described by the following examples. These examples, while illustrating certain specific aspects of the invention, do not portray the limitations or circumscribe the scope of the disclosed invention.

### Example 1

### Isolation of PDE10A

Wild-type (B6CBAF1) and HD transgenic [B6CBA-TgN(Hdexon1)62Gpb] mice (Jackson Laboratories) and adult Sprague-Dawley rats (250-300 g; Charles River Laboratories) and were used in this study. The genotype of the mice was determined by PCR amplification of a 100 bp region of the integrated human HD exon 1 transgene using primers corresponding to nts 3340-3459 (5′-AGG GCT GTC AAT CAT GCT GG-3′) and nts 3836-3855 (5′-AAA CTC ACG GTC GGT GCA GC-3′) of clone E4.1 of the human HD gene (Accession number L34020). PCR conditions used are described in Mangiarini et al. (1996). DNA was extracted from a tail clip and an ear punch from each mouse used in this study. Both samples were subjected to PCR genotype analysis. For in situ hybridization analysis, the animals were anesthetized with >100 mg/kg sodium pentobarbital, decapitated, the brains removed and stored at −70° C. prior to sectioning. For RNA isolation, animals were anesthetized, decapitated and the striatum and cortex were excised and stored in liquid nitrogen prior to RNA extraction. Animal care was given according to protocols approved by Dalhousie University and the Canadian Council of Animal Care.

Differential display was used to identify novel mDNA or previously described mDNA whose relative expression levels are altered as a result of the presence of the transgene. Using differential display, the mRNA populations derived from the striatum of 10 week old wild type were compared with age-matched R6/2 transgenic mice. Differential display has been used extensively (>750 references) since its development (Liang and Pardee, 1992) to identify changes in gene expression in cells and in tissues including brain (Douglass et al., 1995; Babity et al., 1997a; Livesey et al., 1997; Berke et al., 1998). Perhaps the most important finding was the demonstration by Qu et al., (1996) that differential display can be used to isolate genes differentially expressed in inbred strains of mice. The power of differential display is that the sequence information obtained can be directly related to the experimental paradigm. Moreover, such sequence information includes sufficient information to identify transcripts and can then lead to experiments that reveal function of the cognate protein in the experimental model.

DNA sequence information of potentially differentially expressed cDNA can be used to generate oligonucleotide probes for in situ hybridization to define the anatomical and temporal patterns of expression of specific transcripts (see Babity et al., 1997a). This technique is especially useful to study changes in steady-state levels of mRNA in heterogeneous tissue such as brain. Brain tissue can be micro-dissected (Babity et al., 1997b). This enabled the present inventors to reduce the requirement for tissue, and hence compare the mRNA populations derived from individual animals for each experimental group.

Thus RT-PCR (Denovan-Wright et al., 1999) was used to identify differences in the patterns of gene expression between the striatum of wild-type and transgenic mice that were hemizygous for the 5′ UTR, exon 1 and part of intron 1 of the human Huntingon's Disease gene. Total cellular RNA was isolated from the striatum and cortex of three 10 week-old wild-type and three 10 week-old R6/2 HD mice (Mangiarini et al., 1996) and used as the template to generate single-stranded cDNA. Total cellular RNA from each animal and tissue was purified using Trizol reagent (Gibco BRL) and the manufacture's protocol. 10 g aliquots of total RNA were treated with RQ1 DNAse-free DNAse (Promega) in the presence of DNAsin (Promega) DNAse inhibitor to remove trace genomic DNA and then converted to single-stranded cDNA. The primers and conditions for PCR amplification follow those of the Delta™ RNA fingerprinting manual (Clontech).

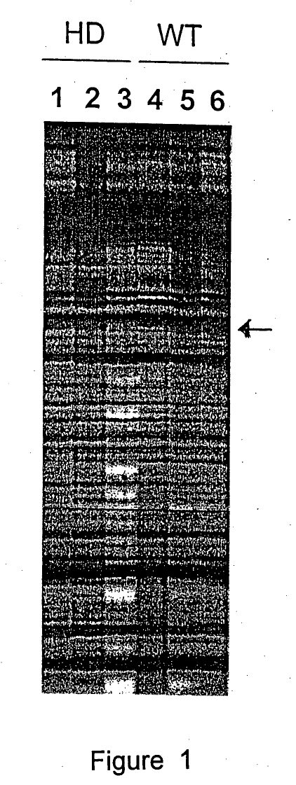

The cDNA was then used as the substrate for PCR reactions using 57 differential display primer combinations. The radio-labelled PCR products were fractionated on a denaturing acrylamide sequencing gels using a Genomyx LR sequencing apparatus, transferred to 3MM filter paper and dried. The dried acrylamide gels were exposed to autoradiography film (BioMax MR™) overnight. After fractionating the radio-labelled PCR products on denaturing acrylamide gels, it was found that the overwhelming majority of the approximately 18,000 PCR products screened were common to both the wild-type and HD mice (data not shown). One PCR product, amplified using the primers P7 (5′-ATT AAC CCT CAC TAA ATG CTG TAT G-3′) and T6 (5′-CAT TAT GCT GAG TGA TAT CTT TTT TTT TCG-3′) of approximately 500 bp, was observed in each of three samples derived from the striatum of wild-type mice (FIG. 1). This 500 bp band was absent from the samples derived from the striatum of the HD mice (FIG. 1) and was absent from each of the samples derived from the cortical tissue (data not shown).

FIG. 1 shows the Down-regulated in Huntington's Disease (PDE10A) transcript, identified by differential display RT PCR. A band of approximately 500 bp (arrow) was amplified from cDNA made form 10 week-old wild-type but not 10 week-old HD striatal tissue. Total RNA from individual animals (numbered 1-6) was used as the substrate for the generation of single-stranded cDNA. Animals 1, 2 and 3 were transgenic HD mice. Animals 4, 5 and 6 were wild-type mice.

### Example 2

### Cloning of PDE10A

The 500 bp band, designate PDE10Apcr, was excised from the dried gel and rehydrated in 40 μL of H₂O for 10 min at room temperature. The eluted DNA was subjected to PCR re-amplification using the P7 and T6 primers, rTaq polymerase (Pharmacia) and the following conditions: 60″@94° C., 19×(30″@94° C., 30″@58° C., 120″@68° C.+4″ per cycle), 7″@68° C. The PCR reaction was subjected to agarose gel electrophoresis and the 500 bp band was removed from the gel, extracted from the agarose using the Qiagen gel extraction protocol and cloned into the vector, pGem-T using standard methods. Plasmid DNA was isolated from selected transformants using Qiagen spin columns. The resultant clone was named pPDE10A.

### Example 3

### Identification of PDE10A

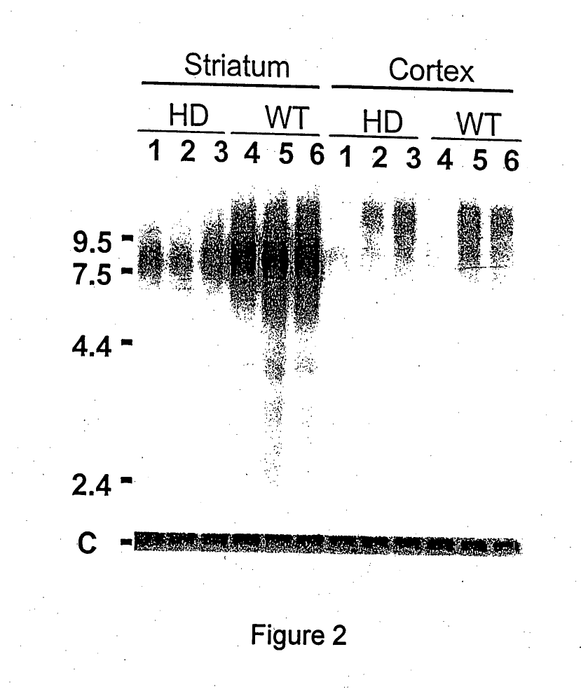

The cloned insert of pPDE10A was radio-labelled and used as a hybridization probe in northern blot analysis (FIG. 2). Northern blots of total RNA were prepared using the method described in Denovan-Wright et al. (1998). The 500 bp cloned insert of PDE10A was radio-labelled with [-³²P]dCTP (3000 Ci/mmol) using the Ready-to-Go dCTP beads (Pharmacia). Northern blot hybridization, brain tissue preparation and in situ hybridization are described in Denovan-Wright et al. (1998). The 500 bp cloned insert of pPDE10A annealed to a transcript of approximately 9.5 kb in total RNA isolated from the striatum of ten week-old wild-type mice.

FIG. 2 demonstrates that PDE10A is expressed in the striatum but not the cortex of wild-type mice and the steady-state levels of PDE10A are reduced in 10 week old transgenic HD mice. The differential expression of PDE10A in HD mice was confirmed by northern blot analysis. The cloned insert of pPDE10A was radio-labelled and used as a hybridization probe in northern blot analysis. The northern blot was prepared by size-fractionating total RNA from the striatum and cortex of three individual 10 week-old HD (1, 2 and 3) and wild-type (4, 5 and 6) mice. Following the hybridization of pPDE10A, the radio-label was removed and the blot was subsequently allowed to hybridize with a probe that detects constituitively expressed cyclophilin. The hybridization pattern of the cyclophilin probe is aligned below the northern blot demonstrating that equivalent amount of RNA were present in each lane. The relative mobility of RNA molecular weight standards (RNA ladder, Gibco BRL) are shown on the left of the northern blot.

The hybridization signal of pPDE10A was significantly lower in the RNA samples derived from the striatum of 10 week-old HD mice. No expression of the PDE10A mRNA was detected in the cortical RNA samples derived from either the wild-type or HD mice.

### Example 4

### Sequencing PDE10A

The sequence of the cloned differential display band, pPDE10A, was determined using M13 universal forward and reverse sequencing primers and the T7 sequencing kit (Pharmacia). The 484 bp cDNA fragment did not have sequence similarity to any Genbank entries.

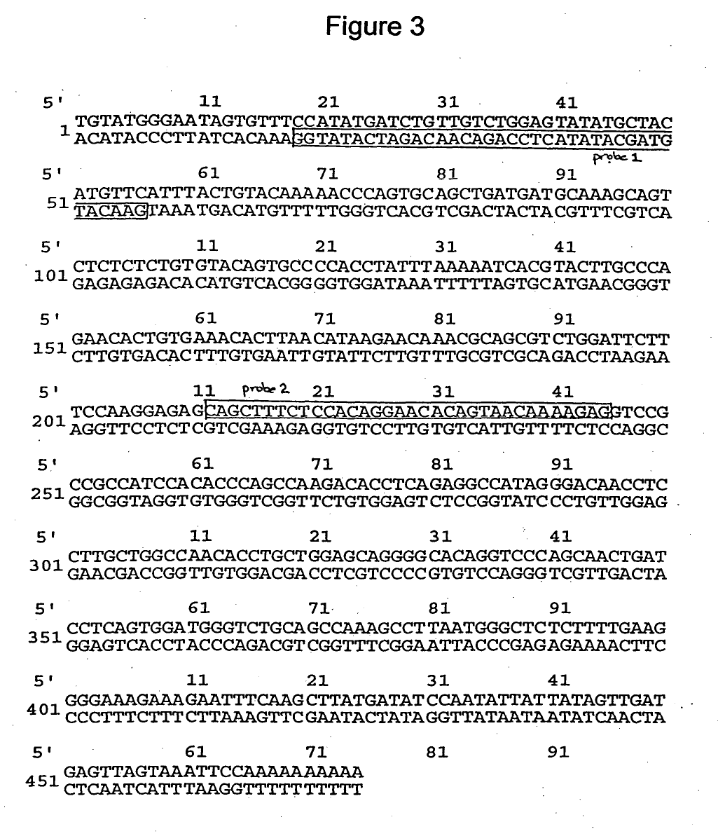

FIG. 3 shows the nucleotide sequence of the cloned PDE10A differential display product, pPDE10A. The position of the primers used to amplify the fragment are underlined and labelled. The nucleotide sequence and position of oligonucleotide probes 1 and 2 within the pPDE10A sequence are shown.

### Example 5

### Isolation and Characterization of cDNA PDE10A

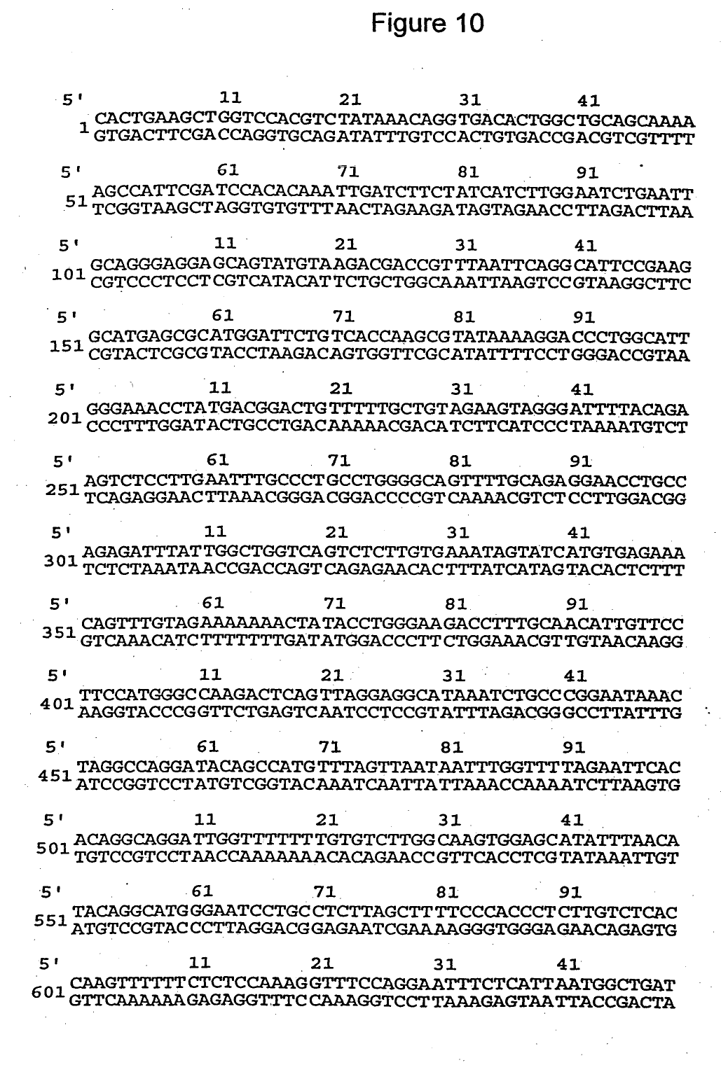

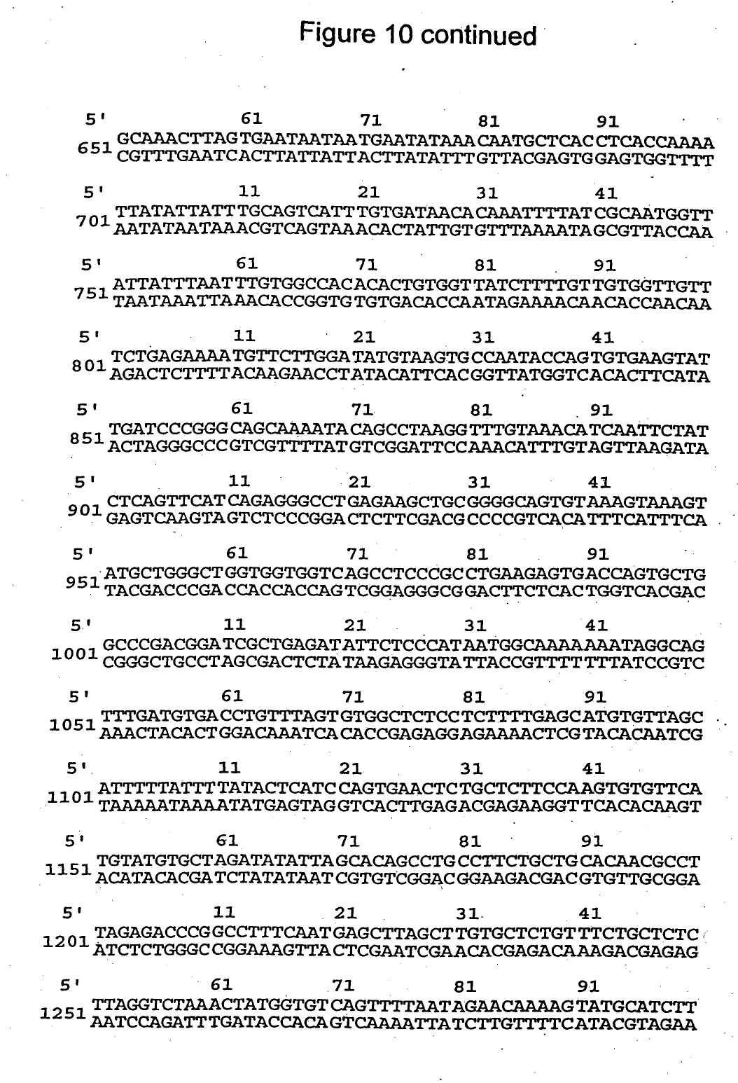

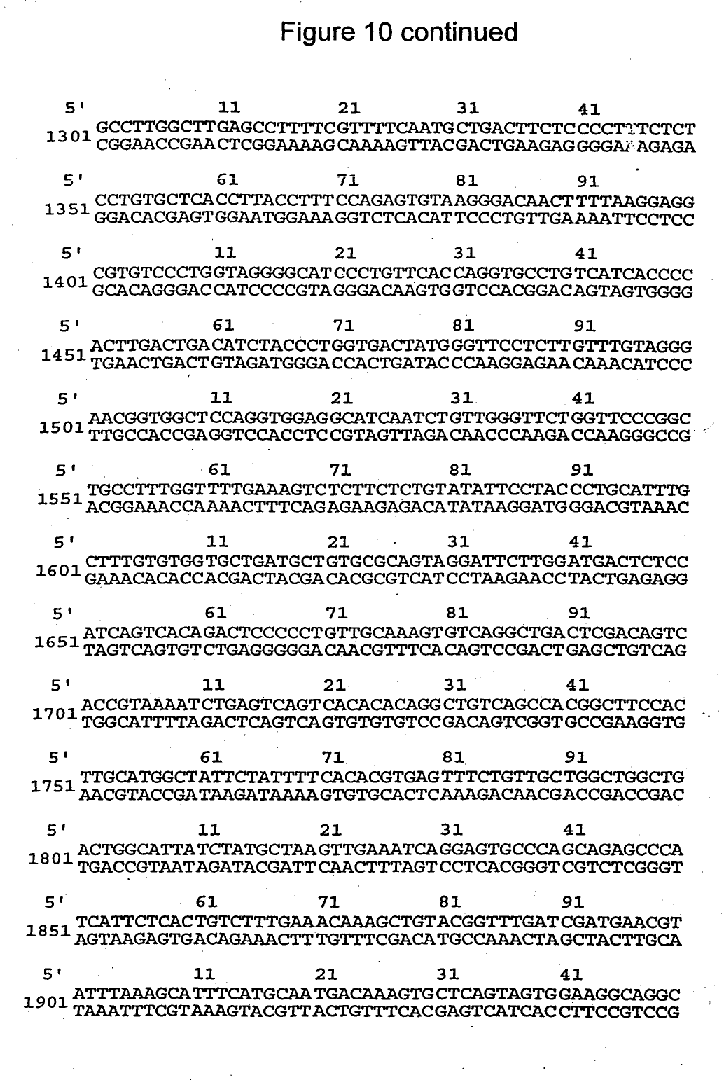

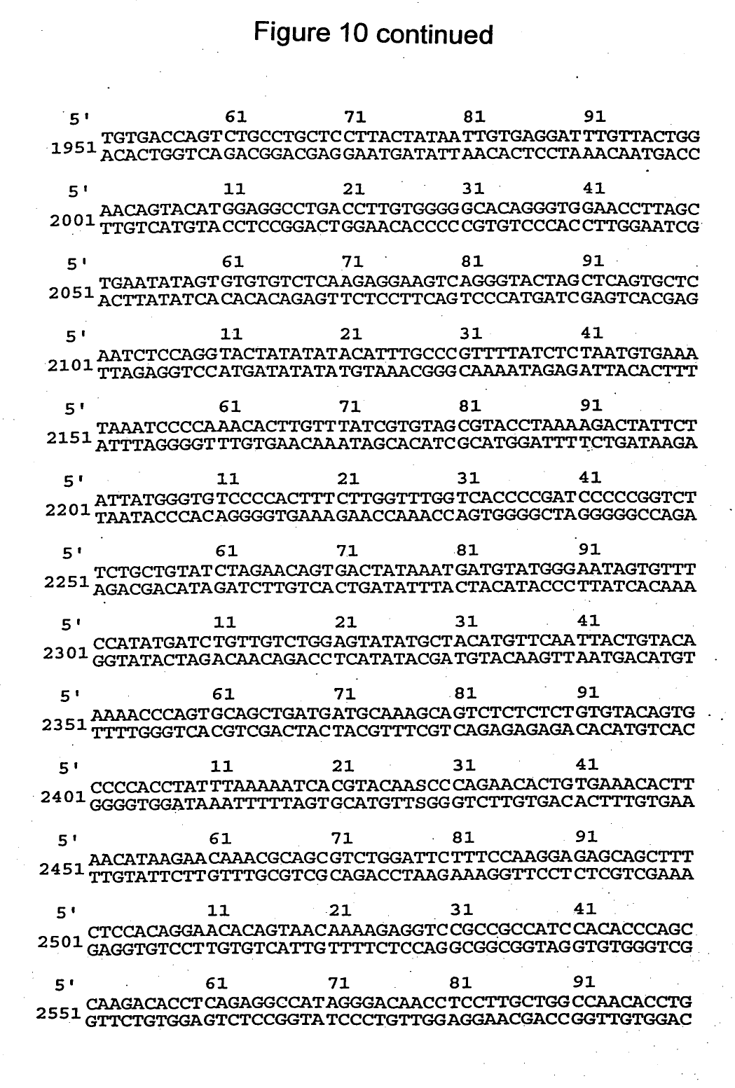

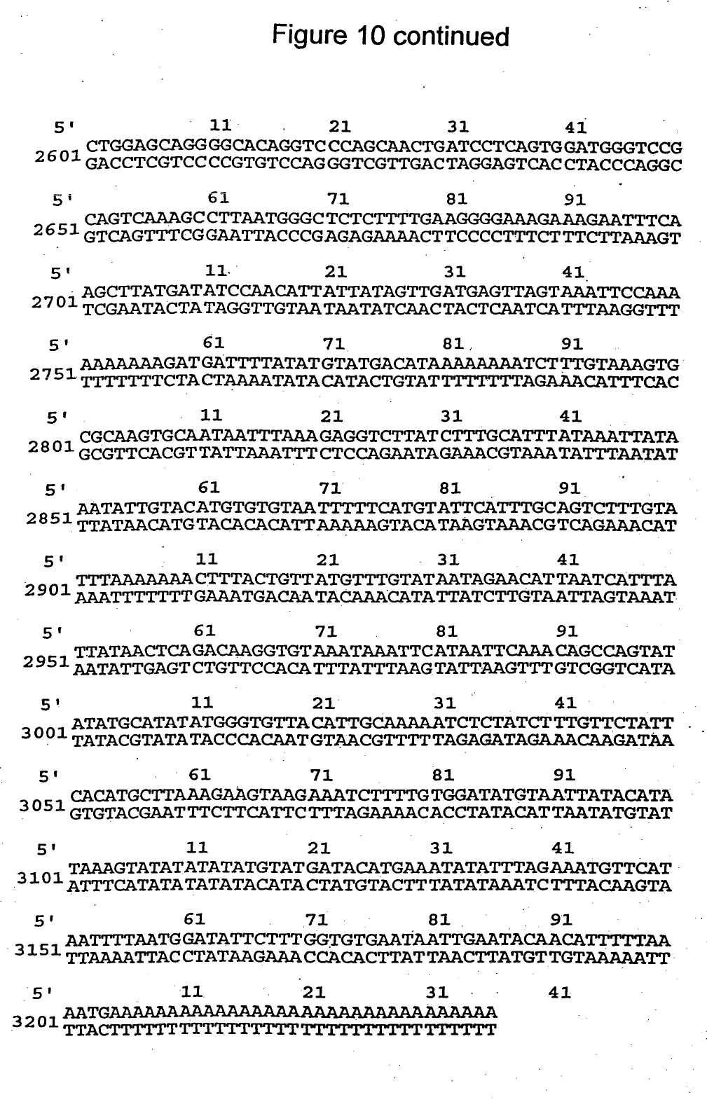

In order to isolate PDE10A cDNA clones, oligonucleotide probes 1 and 2 were used in 5′ and 3′ Rapid Amplification of cDNA Ends (RACE) reactions using commercially prepared RACE-ready mouse striatal cDNA (Clontech). Several independent clones were isolated and those that contained the sequence of pPDE10A were selected for further analysis. Each of the 5′ RACE clones was identical in sequence over the length that the clones could be aligned. The difference in length between these clones is a result of termination of the original reverse-transcriptase reaction at different positions along the mRNA. No difference in size or sequence was detected between several 3′ RACE clones. The longest 5′ RACE clone and one 3′ RACE clone were completely sequenced using internal primers. The present inventors were able to isolate a very short clone that extended the 5′ RACE clone using an internal primer (probe 3, 5′-CTA TTT CAC AAG AGA CTG ACC AGC CAA TAA ATC TC-3′). The compiled sequence of the first PDE10A cDNA clone, named cPDE10A-1 is presented in FIG. 10. cPDE10A-1 is 3235 bp in length. The restriction map of cPDE10A-1 is shown in FIG. 11.

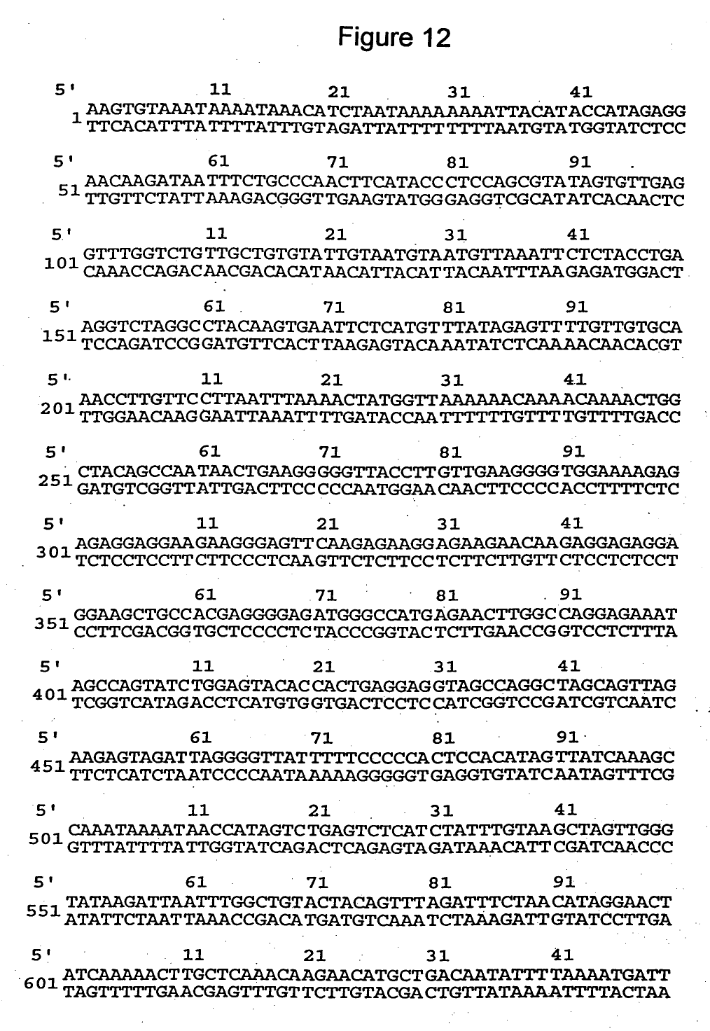

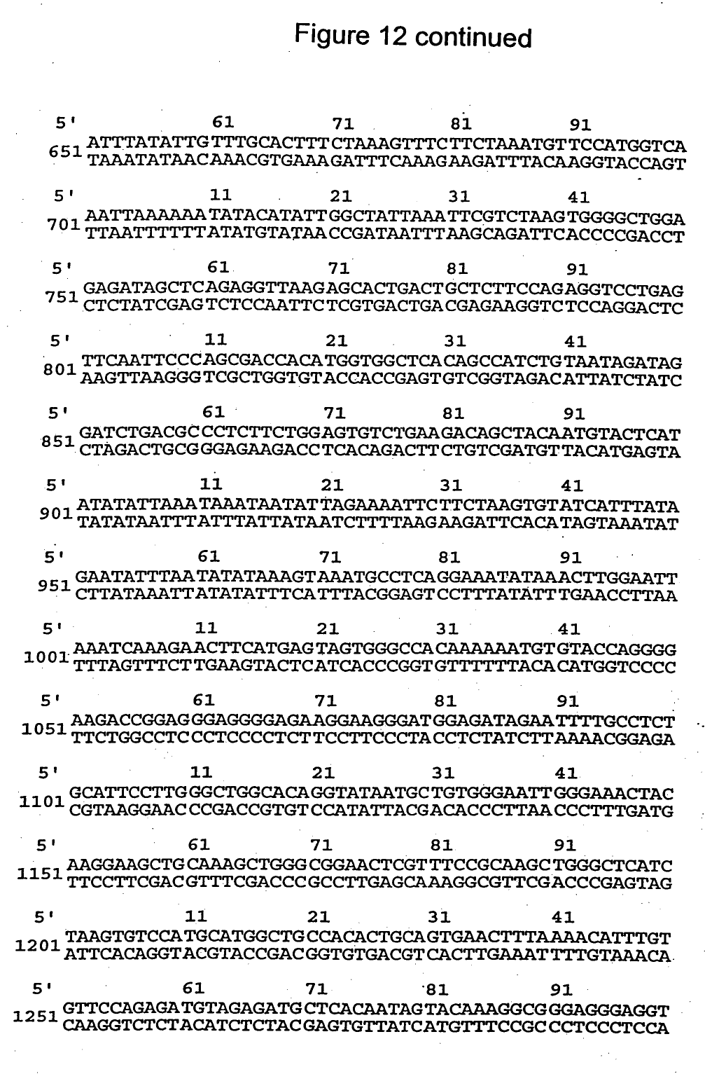

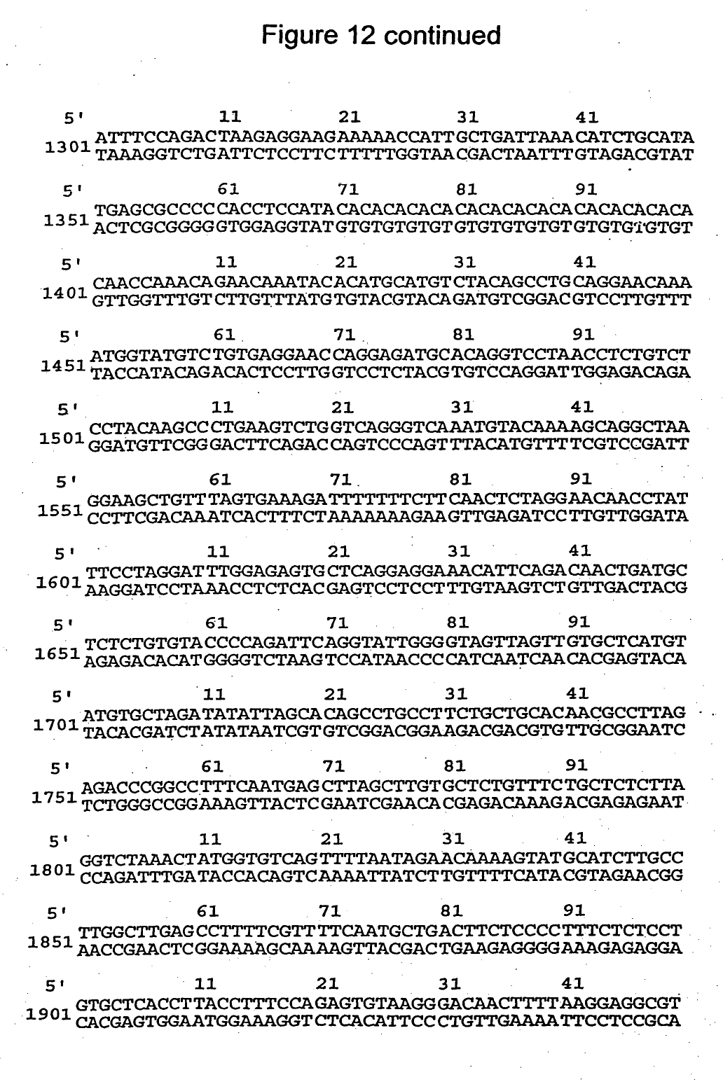

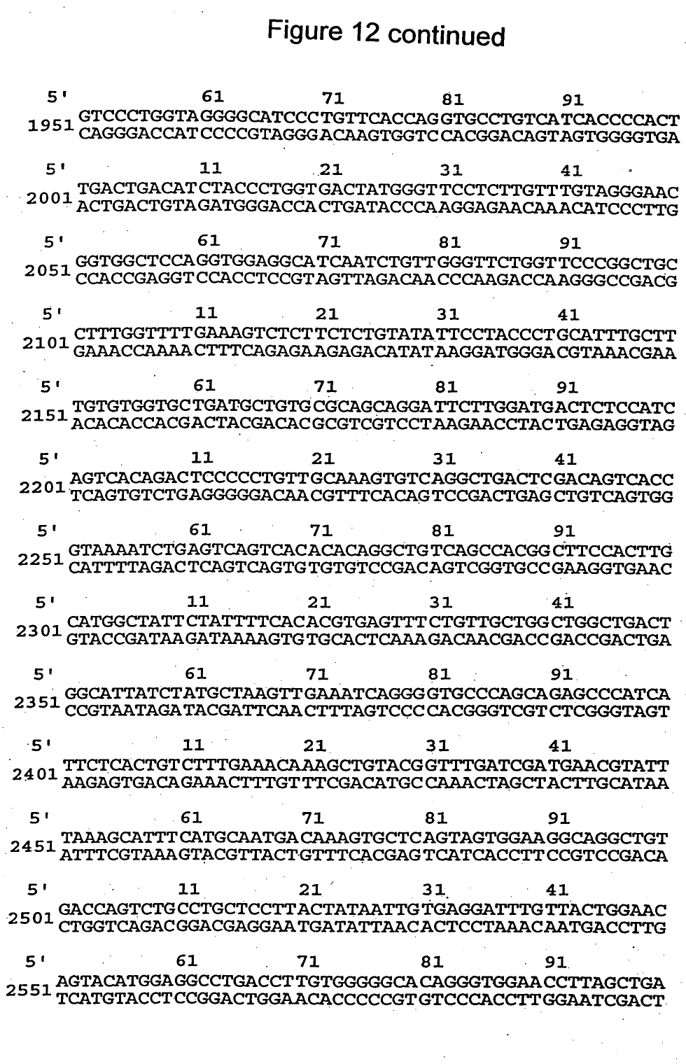

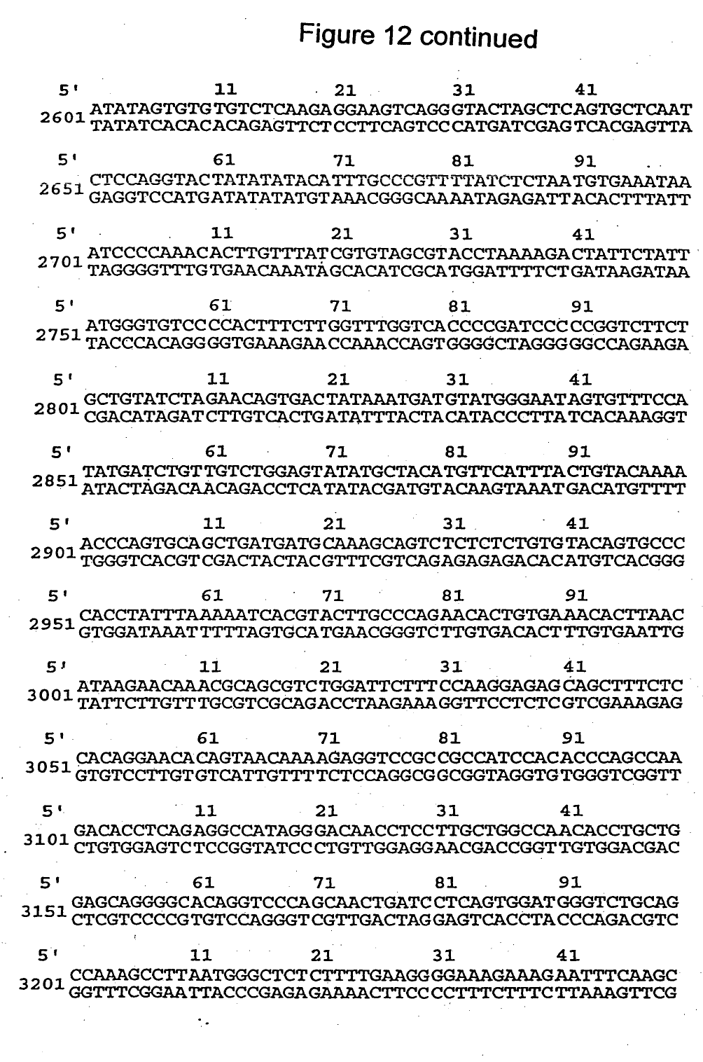

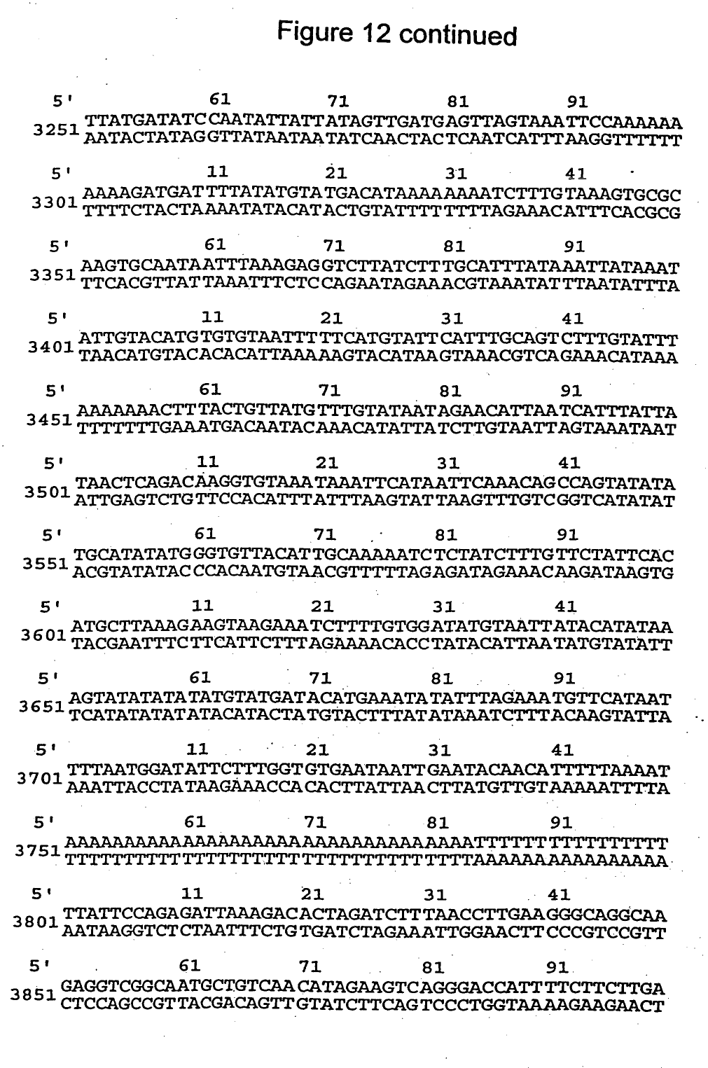

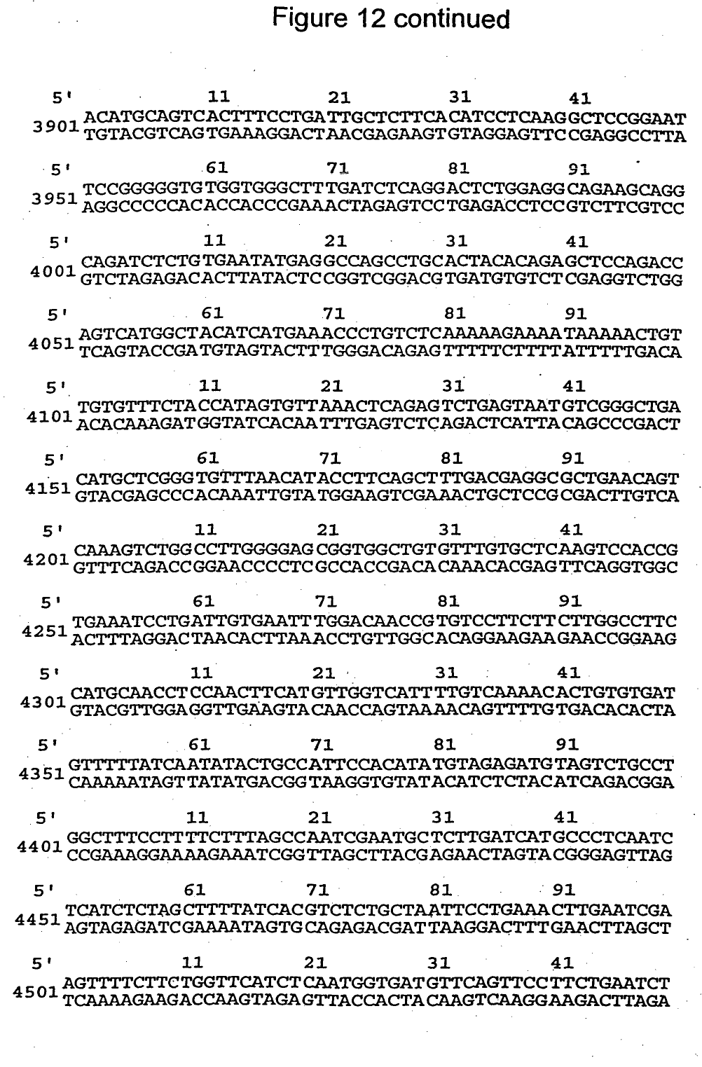

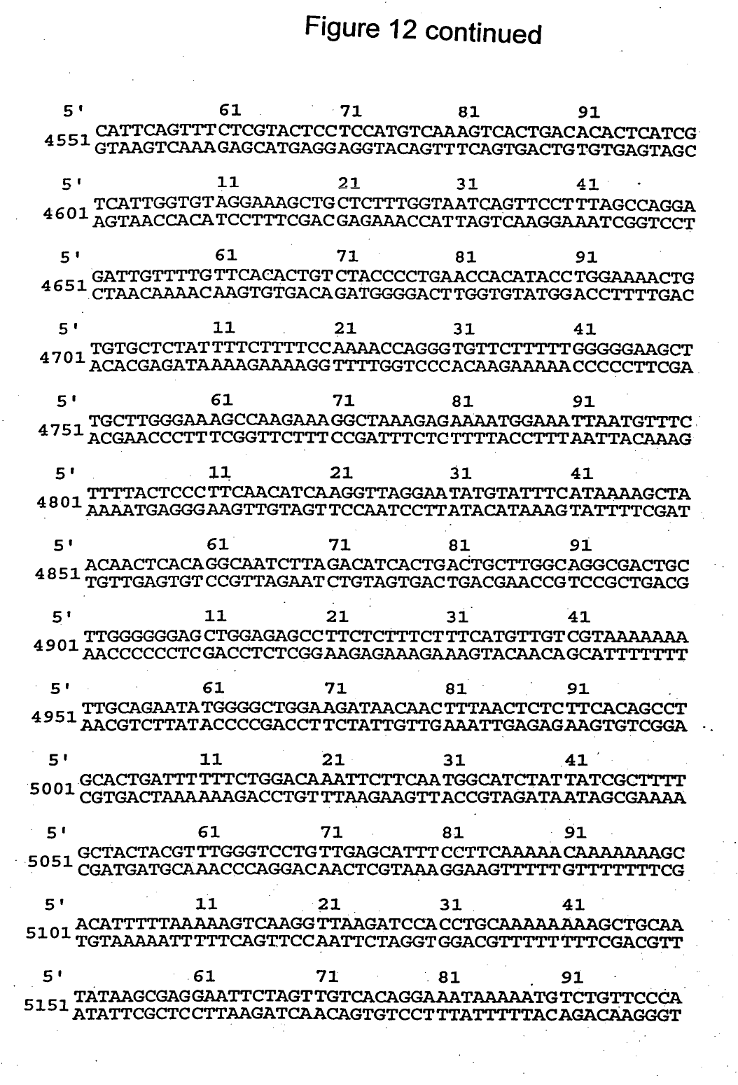

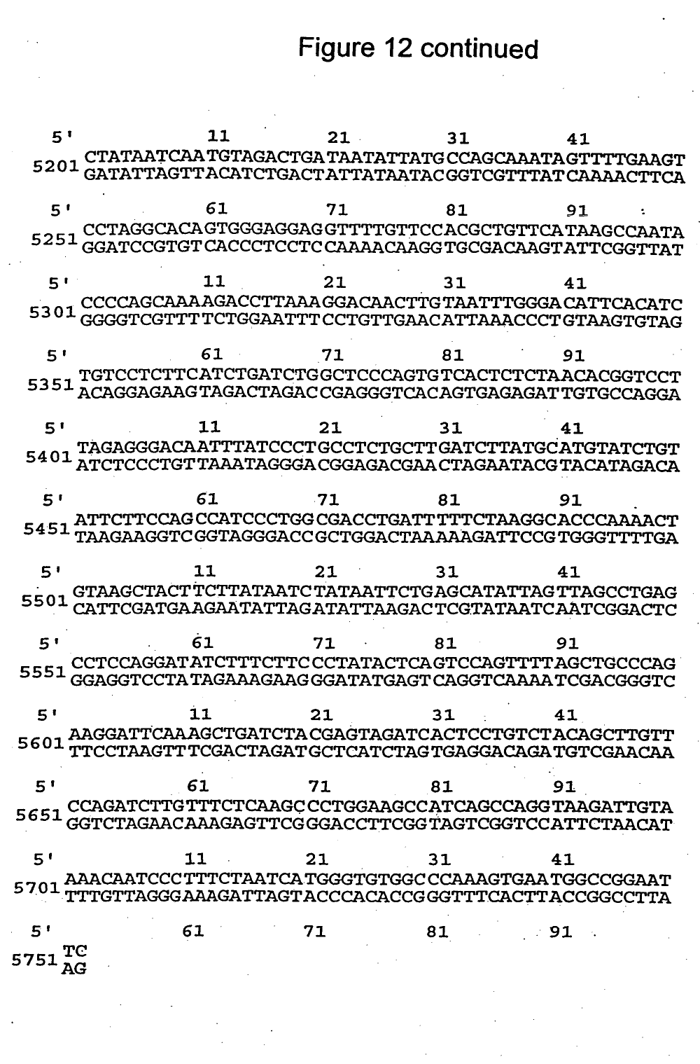

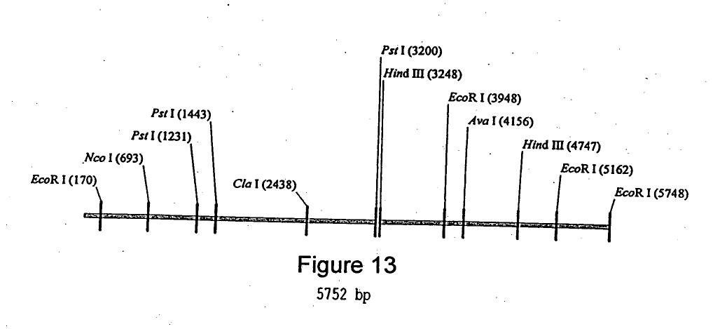

The mRNA that hybridized with pPDE10A was approximately 9.5 kilobases in length. In order to obtain PDE10A cDNA clone that was larger than cPDE10-1, the present inventors screened a mouse brain cDNA library. Several clones were identified that hybridized with the pPDE10 probe. The sequence of the largest of these cDNA clones, cPDE10-2, was determined. The sequence (FIG. 12) was 5753 base pairs in length. The restriction map of cPDE10-2 is shown in FIG. 13.

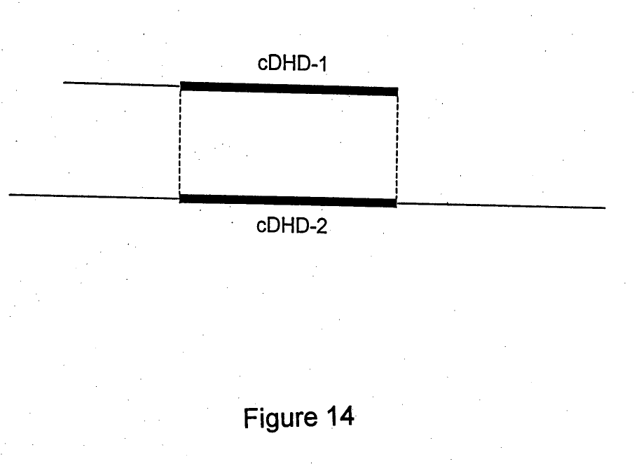

cPDE10-1 and cPDE10-2 share sequence identity over 2095 bp. However, the 5′ 1142 bp of cPDE10-1 and the 5′ 1689 bp of cPDE10-2 are unique to each clone. Clone cPDE10-2 extends 1969 bp in the 3′ direction compared to cPDE10-1. A schematic showing the regions of sequence identity and the unique sequences of cPDE10-1 and -2 are shown in FIG. 14.

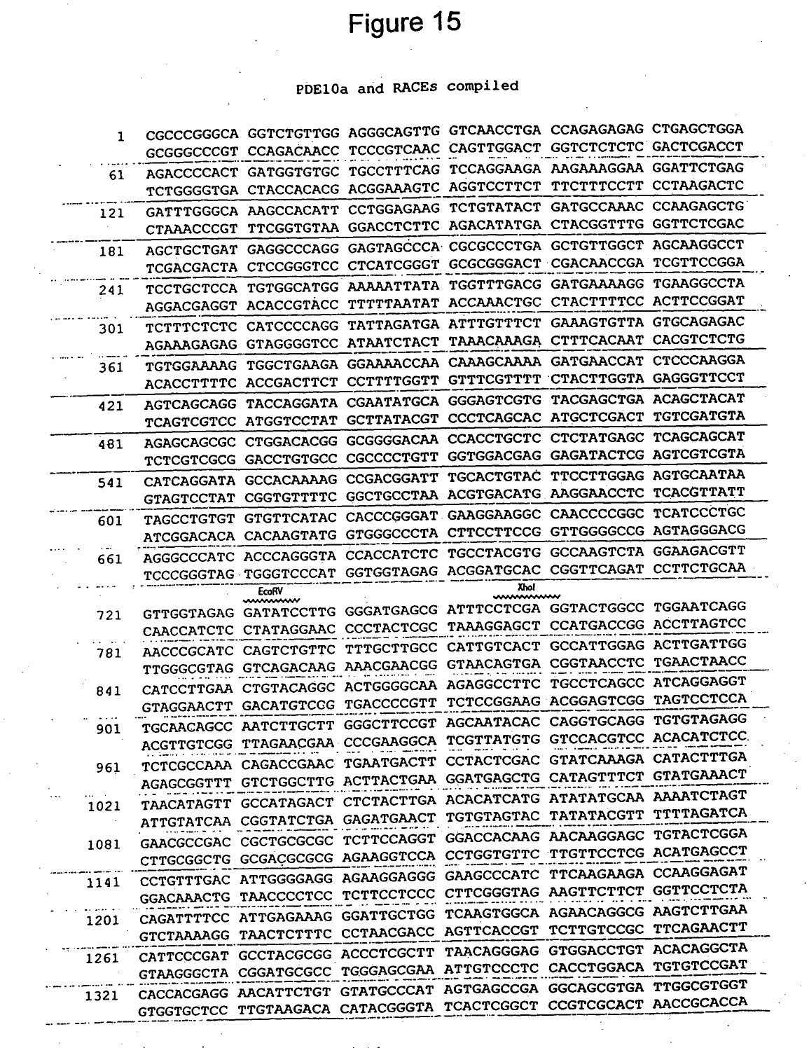

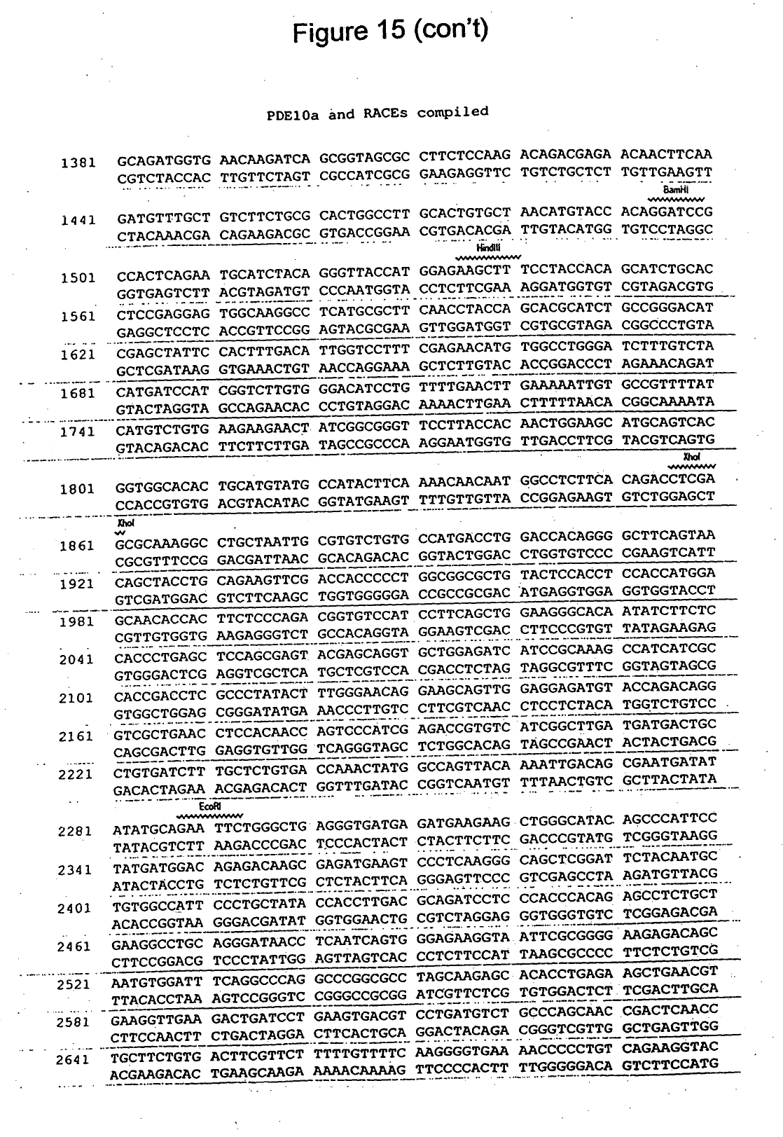

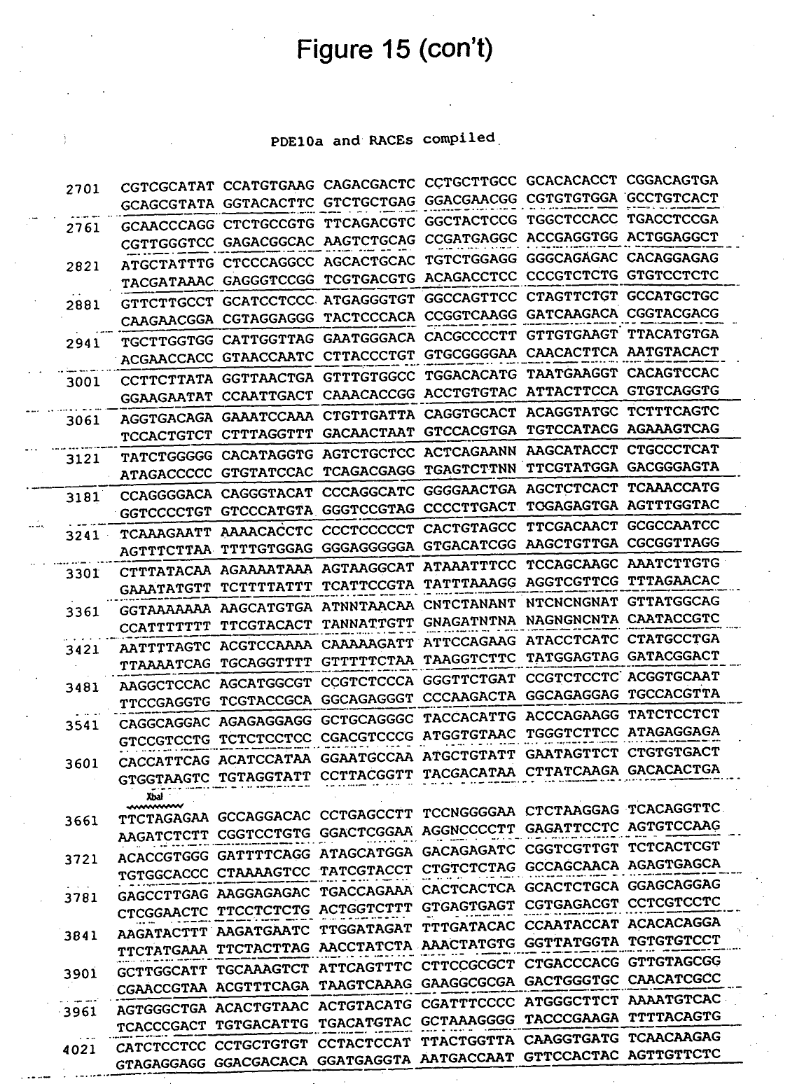

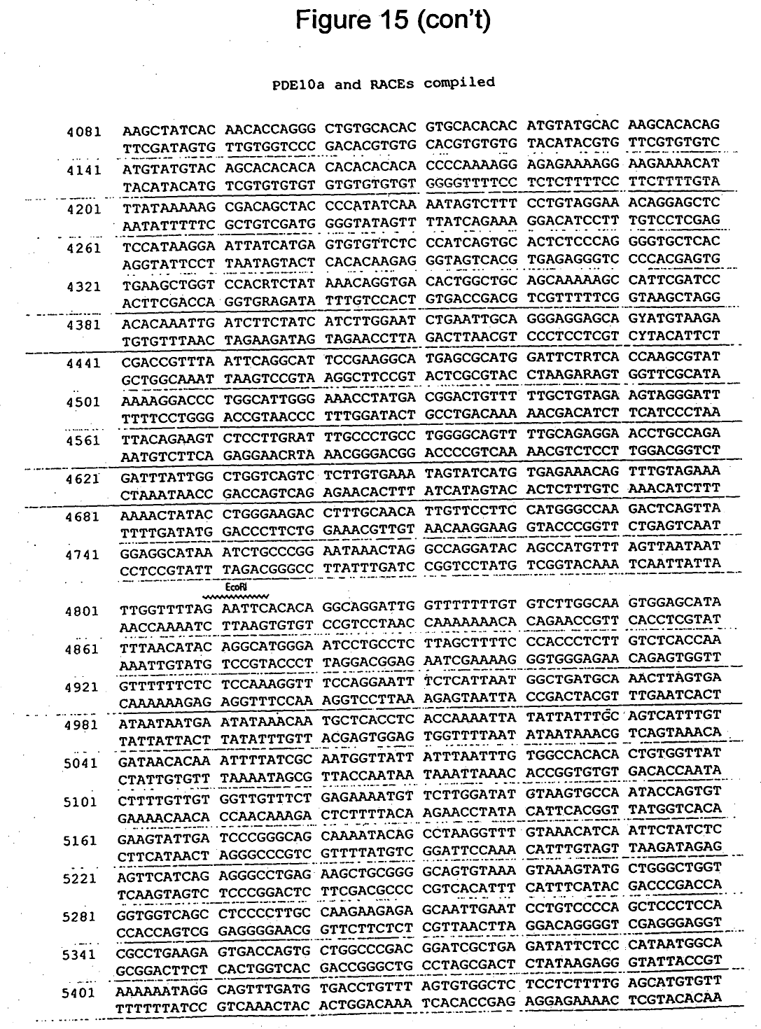

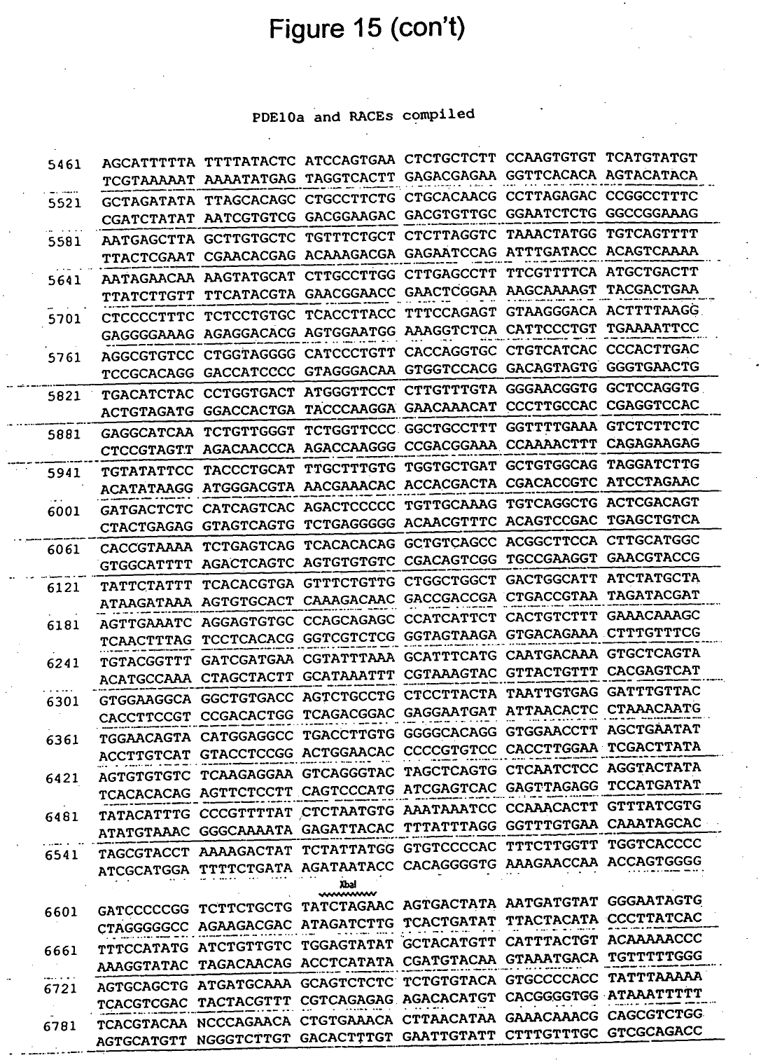

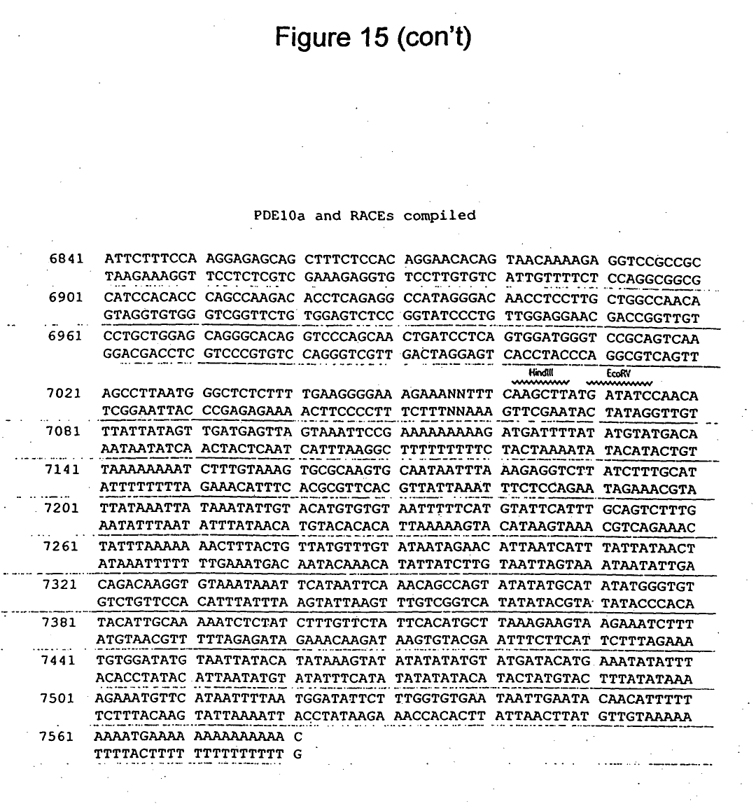

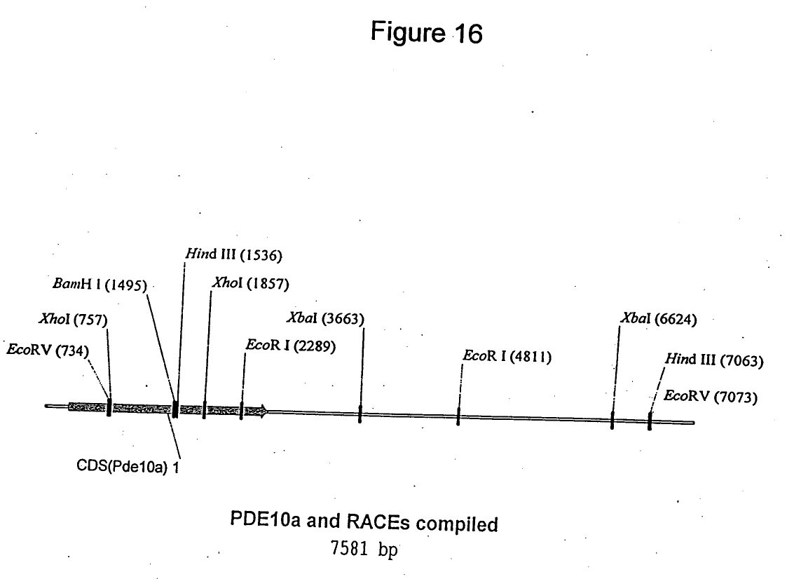

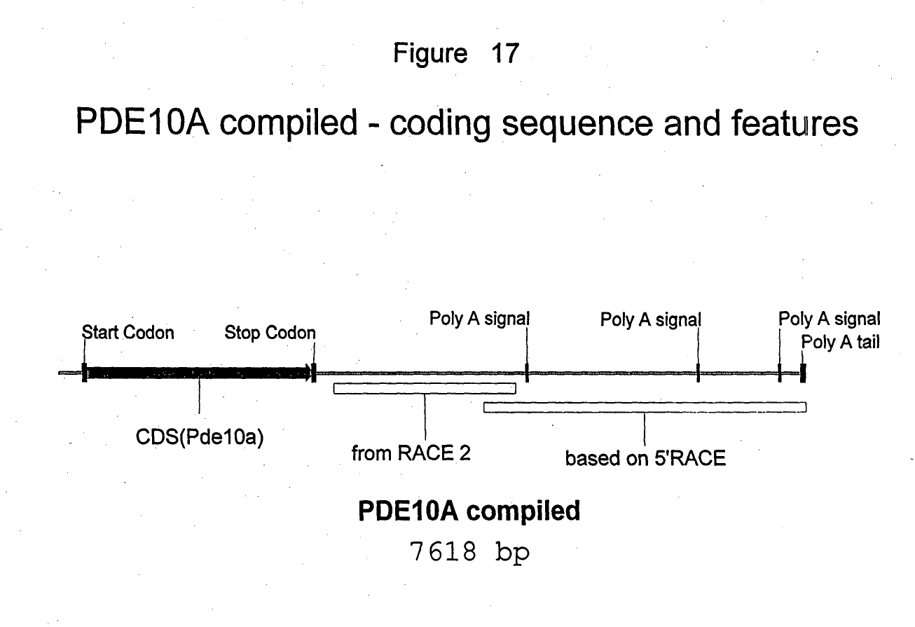

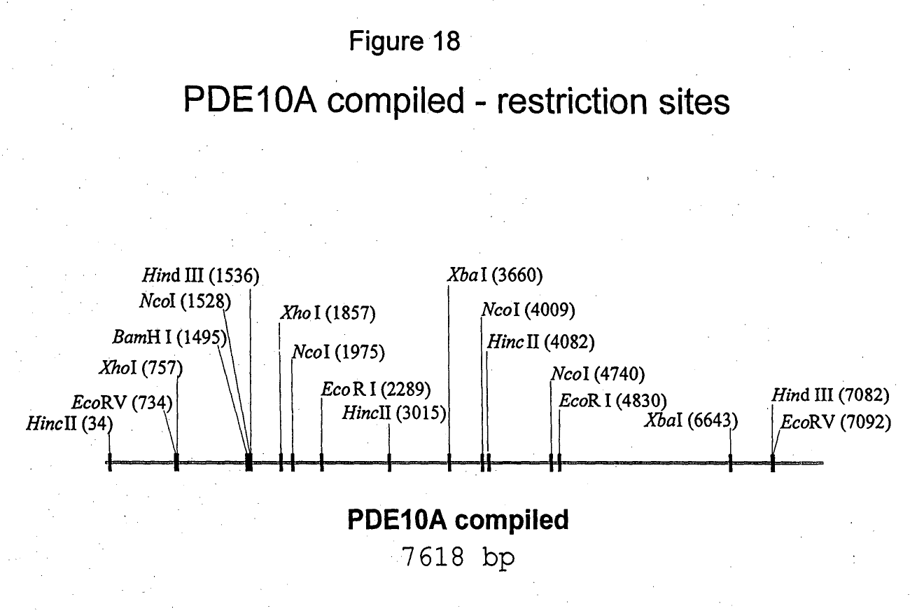

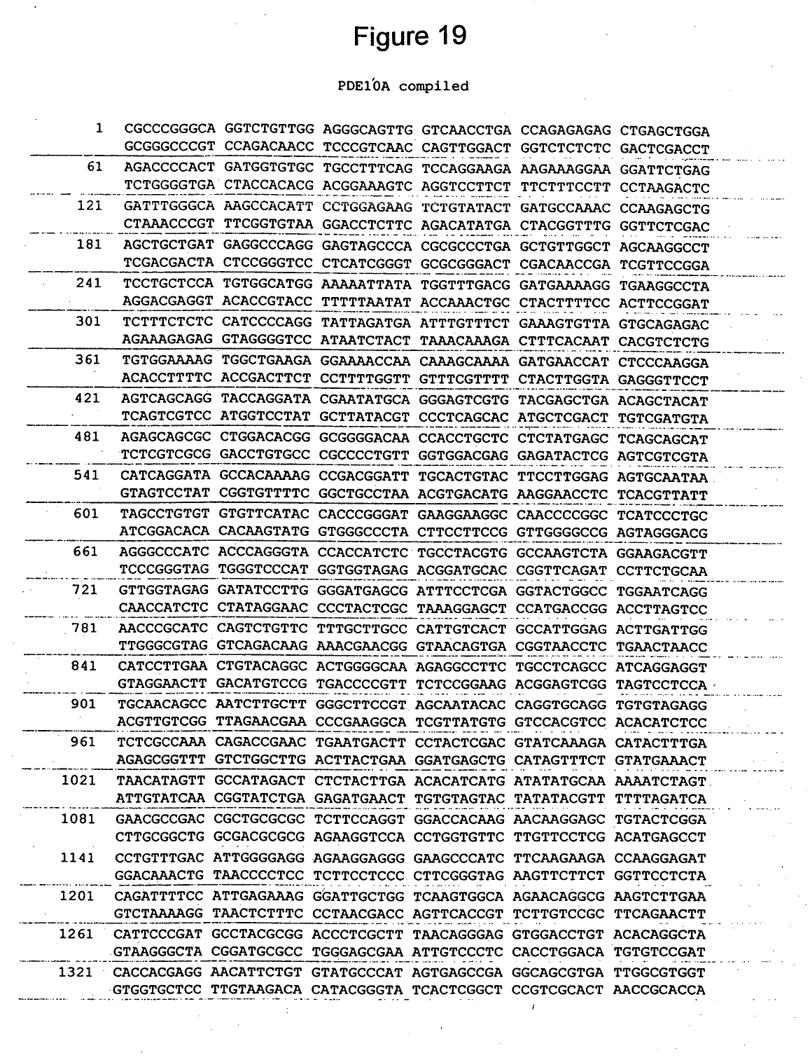

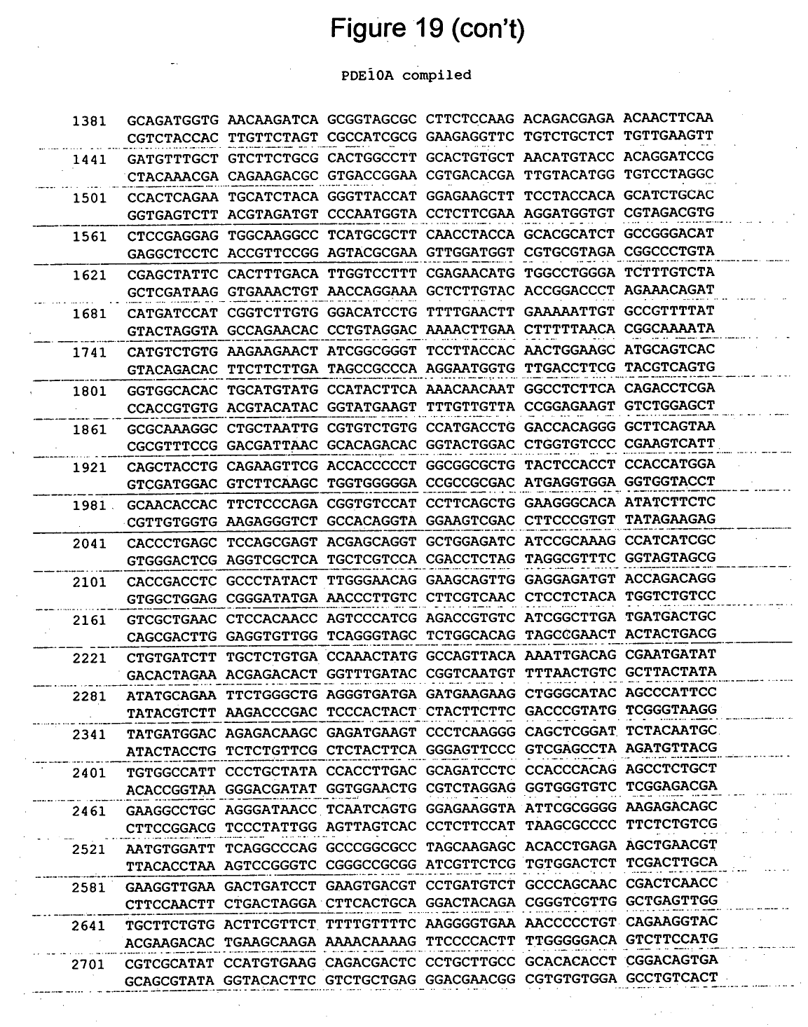

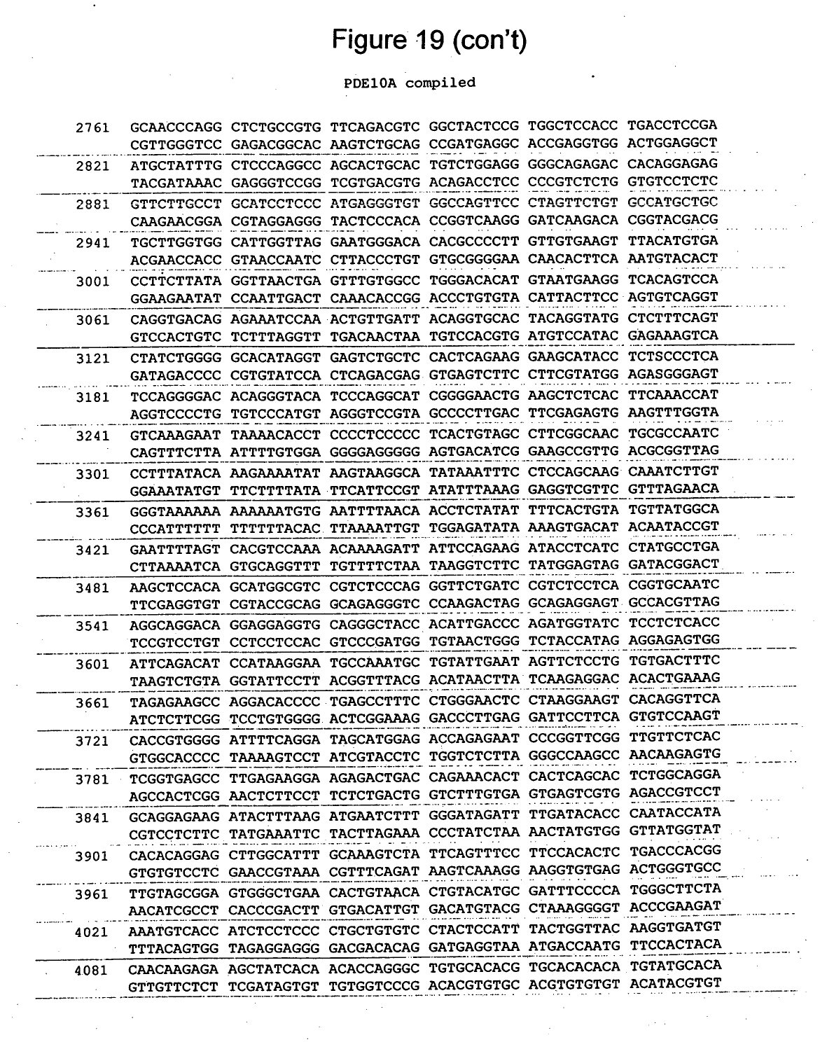

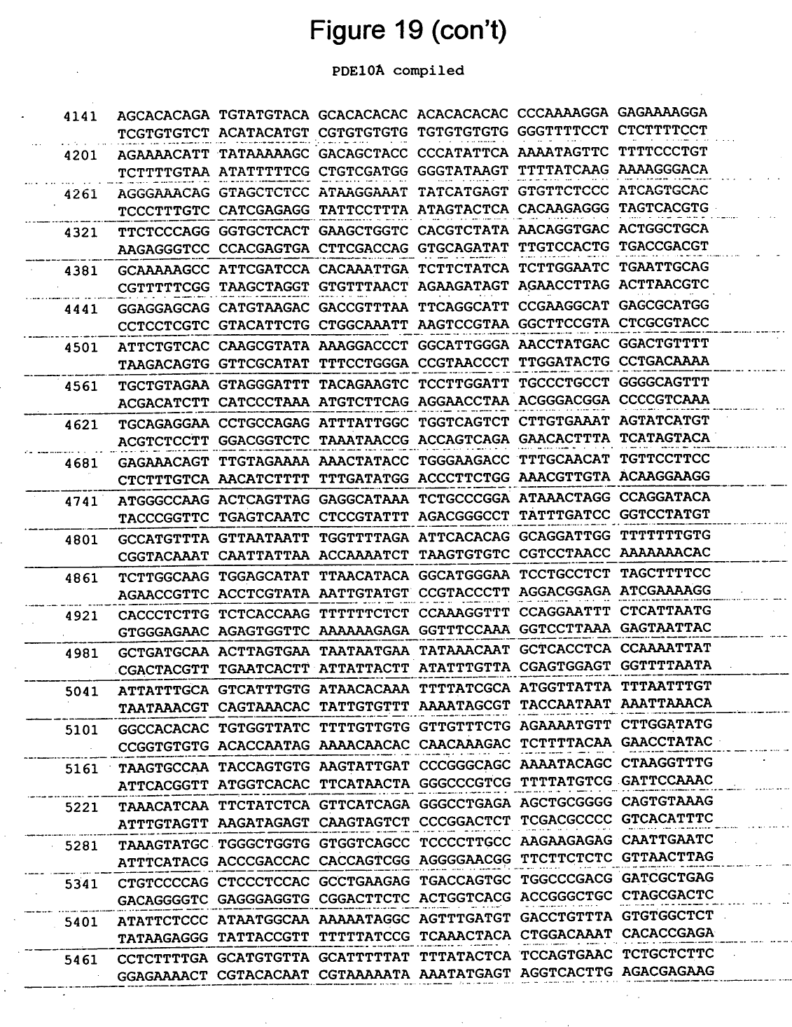

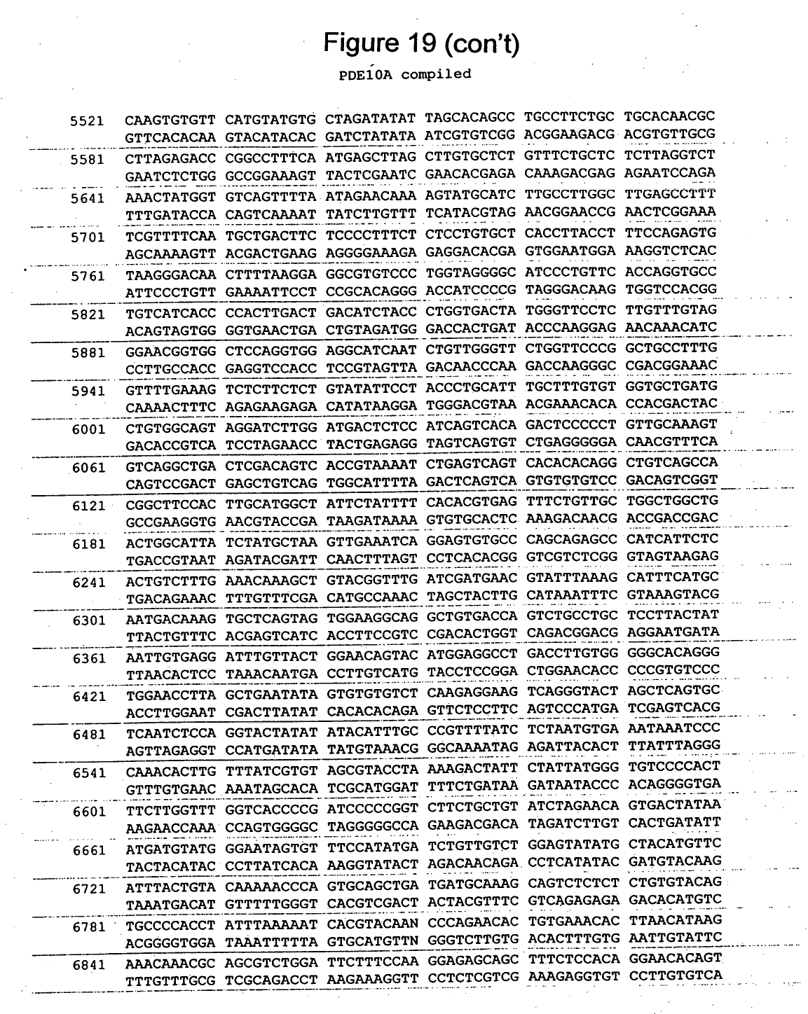

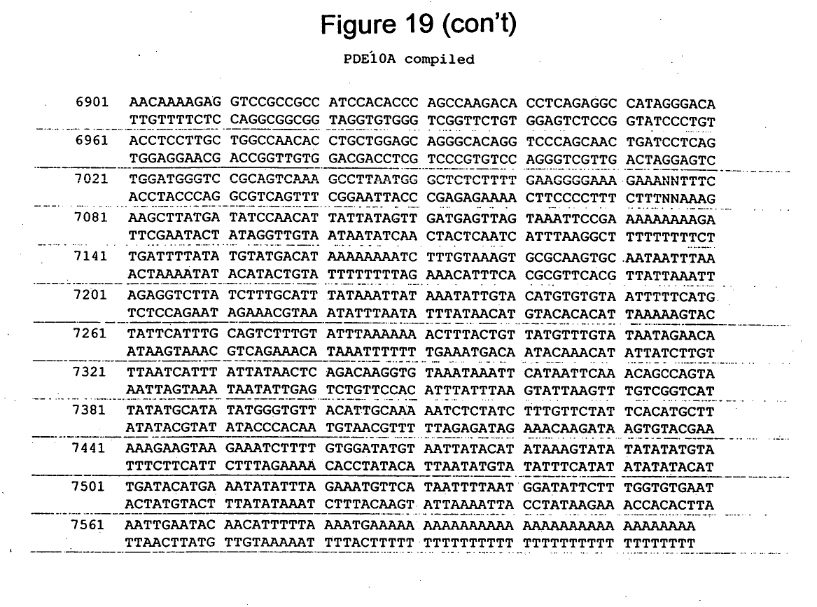

The compiled sequence of the mouse PDE10 cDNA clone, named cPDE10A, is presented in FIG. 15 with RACEs. A further sequence, without RACEs, is shown in FIG. 19. The coding sequence and restriction map of cPDE10A is shown in FIG. 16, and updated at FIG. 17. FIG. 18 is a restriction map of PDE10A. The coding region has a met initiator commencing at nucleotide 257, with a stop codon ending at nucleotide 2596.

PDE10A was found to have extremely high homology with human PDE10s identified by Loughney et al., WO99/42596, the contents of which are incorporated herein by reference.

### Example 6

### Localization of PDE10A in the Brain

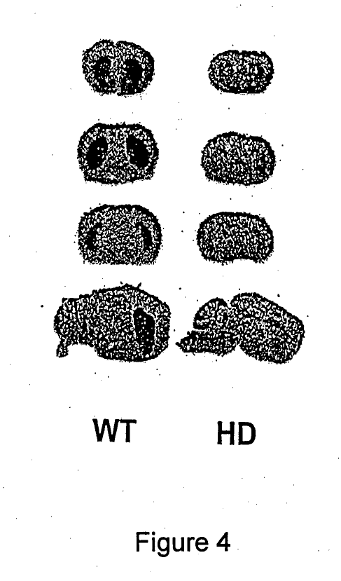

In order to identify the coding strand and to localize the transcript in the wild-type mouse brain, two oligonucleotide probes were designed (probe 1, 5′-GAA CAT GTA GCA TAT ACT CCA GAC AAC AGA TCA TAT GG-3′; probe 2, 5′-CAG CTT CTC CAC AGG AAC ACA GTA ACA AAG AG-3′) that were complementary to different regions and strands of the 484 bp pPDE10A clone. These oligonucleotides were used for in situ hybridization analysis. Using high stringency post in situ hybridization washes (2×30′ in 1×SSC@58° C., 4×15′ in 1×SSC@58° C., 4×15′ in 0.5×SSC@58° C., 4×15′ in 0.25×SSC@58° C.), it was found that oligonucleotide probe 1 annealed with mRNA in the striatum, nucleus accumbens and olfactory tubercule of ten week-old wild-type mice (FIG. 4). The hybridization signal was significantly reduced in the striatum, nucleus accumbens and olfactory tubercle of the 10 week-old HD mice (FIG. 4).

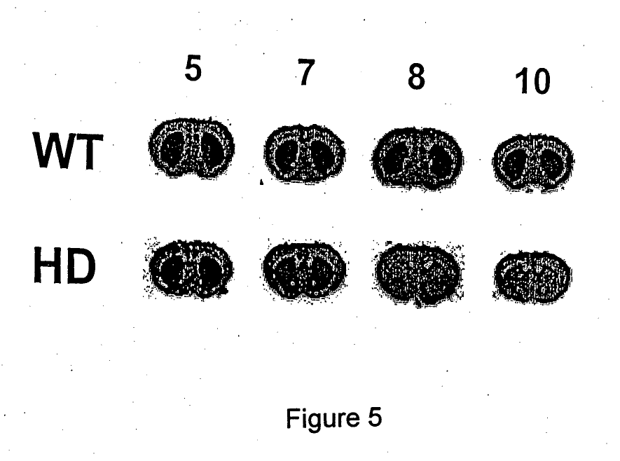

FIG. 5 shows in situ hybridization of probe 1 to coronal (top three sections) and saggital (bottom section) 10 week-old wild-type (WT) and HD mouse brain sections. Specific hybridization of the probe was observed in the striatum, nucleus accumbens and olfactory tubercle of wild-type mice. The top three sections represent the distribution of PDE10A throughout the rostral-caudal axis of the striatum.

The in situ hybridization results confirmed the northern blot analysis demonstrating, 1) that the expression of PDE10A mRNA was restricted to the striatum, nucleus accumbens and olfactory tubercle and 2) that the levels of PDE10A mRNA were decreased in HD mice compared to the wild-type. The probe did not anneal with mRNA in any other brain nuclei. No hybridization of oligonucleotide probe 2 was observed in any region of the brain in wild-type or HD mice (FIG. 3). Based on this hybridization, the coding strand, complementary to probe 1, of pPDE10A was defined.

### Example 7

### Characterization of PDE10

The in situ hybridization using oligonucleotide probe 1 demonstrated that PDE10A mRNA levels in the striatum, nucleus accumbens and olfactory tubercule were decreased in ten week-old HD mice. By ten weeks of age, the HD mice all showed motor symptoms including resting tremor and stereotypic involuntary movements. Moreover, these mice immediately clasped their feet together and curled into a tight ball when picked up by their tails.

As the phenotypic signs are progressive over a number of weeks, the present inventors examined whether the PDE10A transcript was ever expressed in the striatum of the HD mice or whether the steady-state levels of the transcript diminished in the striatum in a course that parallelled the development of the motor disorders. Wild-type and HD mice were sacrificed at 5, 7 and 8 weeks of age and their brains were prepared for in situ hybridization analysis using probe 1 (FIG. 5).

FIG. 5 shows the levels of PDE10A mRNA decrease in HD mice over the period of time that the HD mice develop abnormal movements and postures. In situ hybridization analysis of coronal and saggital sections of wild-type and HD mouse brain using oligonucleotide probe 1 which is complementary to the coding strand of PDE10A. At 5 weeks of age, before the development of motor symptoms, the HD mice express the PDE10A transcript in the same brain nuclei and at the same relative levels as wild-type mice. The steady-state level PDE10A decreases in the striatum, nucleus accumbens and olfactory tubercle from 5 to 10 weeks in the HD but not wild-type mice. By 9 weeks of age, the HD mice have abnormal movement and posture. The numbers refer to the age in weeks of the wild-type (WT) and Huntington's (HD) transgenic mice.

None of the mice at these ages had overt motor symptoms. Sections taken throughout the rostral-caudal axis of the striatum showed that PDE10A was expressed in the 5 week-old wild-type and HD mice. The relative hybridization of probe 1 did not change in 5, 7, 8 and 10 week-old wild-type mice. The intensity of the hybridization signal appeared to decrease in the striatum, nucleus accumbens and olfactory tubercle of HD mice from 5 to 10 weeks compared to their wild-type litter mates (FIG. 5).

The levels of PDE10A were significantly reduced by 8 weeks of age in the HD mice, using two in situ oligonucleotide probes, one complementary to the 3′ UTR, the second complementary to an internal portion of the coding region. The hybridization pattern observed in the wild-type and HD mice was the same for both the probes employed. This analysis demonstrated that there is a reduction in the complete PDE10A mRNA levels during the development of the HD phenotype and not that there was a differential reduction in the PDE10A coding region as compared to the extensive 3′ UTR. Moreover, in situ hybridization using the PDE10A-specific probe against neurologically normal and HD human brain tissue demonstrated that there was a decrease in PDE10A levels in human HD patients.

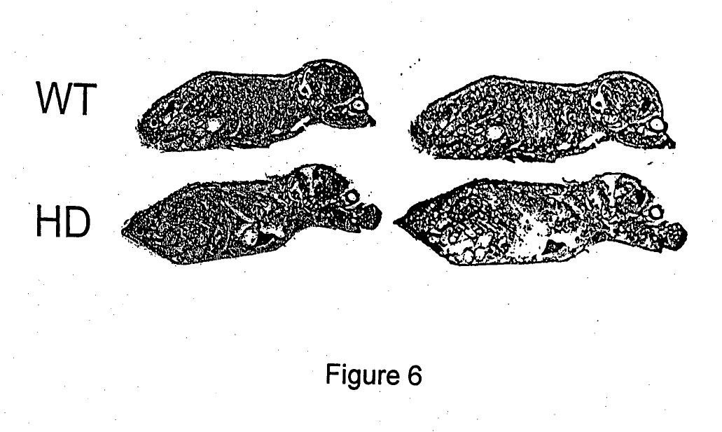

One day old wild-type and HD mice were frozen, sectioned on a cryostat and whole mouse sections were prepared for in situ hybridization using probe 1. The same high stringency post-hybridization washing conditions were employed for the one day-old mouse body sections as were used for the adult mouse brain sections. Parallel in situ hyridization experiments using the probe 2 were performed in order to determine the level of non-specific signal in the mouse sections. Probe 1 specifically annealed to the developing striatum (FIG. 6).

FIG. 6 demonstrates that PDE10A is expressed in the developing striatum of one day-old wild-type and HD mice. The sections on the left were subjected to in situ hybridization using probe 1. Following hybridization, the sections were counter-stained with cresyl violet to visualize the mouse organs. The signal outside the brain was non-specific as probe 2 and other unrelated control oligonucleotide probes all labelled these tissues.

There was no difference in the pattern of hybridization between the one day-old wild-type and HD mice demonstrating that PDE10A was expressed in the developing brain of both wild-type and HD mice.

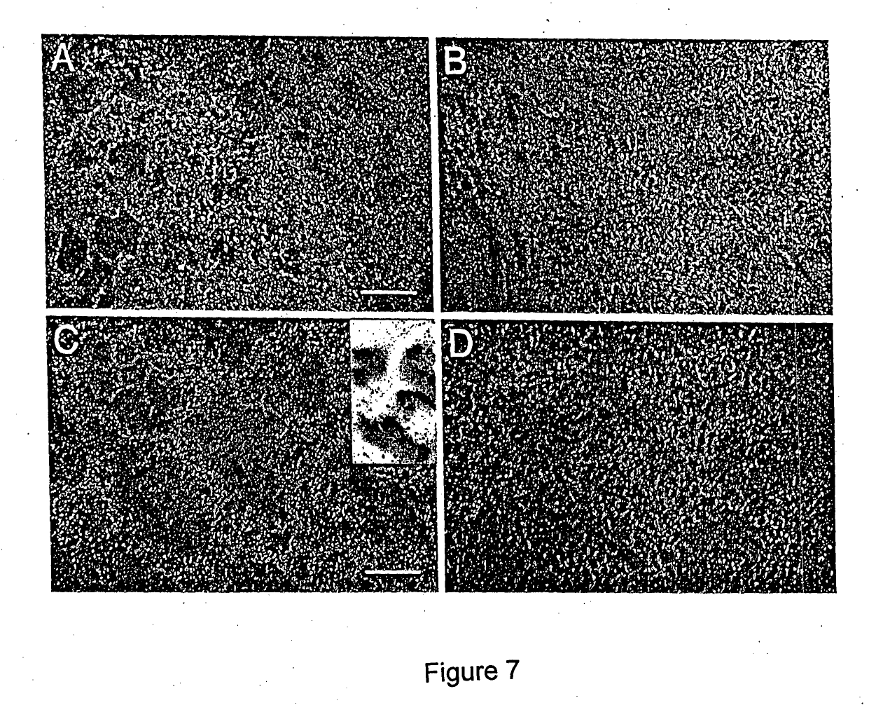

Following in situ hybridization, the sections were covered in autoradiographic emulsion, left in the dark to expose for 4 weeks and then developed and viewed under dark-field microscopy or, after counter-staining the sections with cresyl violet to visualize neuronal cell bodies, under bright-field microscopy. Silver grains were observed to be concentrated in the striatum of the wild-type mice. FIG. 7 shows emulsion autoradiography of mouse brain sections following in situ hybridization of probe 1 demonstrated that the PDE10A transcript is expressed in neurons. PDE10A is not homogeneously distributed throughout the mouse striatum. Dark field illumination of the sections after emulsion autoradiography showed that the silver grains were clustered in specific regions of the 10 week old wild-type mouse striatum (A and C). Sections from 10 week old HD mice subjected to identical in situ and emulsion autoradiographic conditions are shown in B and D. The photomicrographs shown in A and B were viewed using the 10× objective (bar represents 100 μm). The micrographs shown in C and D, were viewed under the 20× objective (bar represents 25 μm). The insert in panel C is a portion of the section in A and C counter-stained with cresyl violet to visualize the neurons, viewed using the 40× objective under bright filed illumination. Note the distribution of the silver grains over some, but not all, of the striatal neurons as well as being concentrated around clusters of neurons. It appeared that the silver grains were absent from fibre tracks within the striatum. It appeared that PDE10A mRNA was not confined to regions close to the nucleus but was dispersed in cellular processes.

Huntingtin with an expanded polyglutamine tract (htt-HD) is expressed in neurons of the brain and body throughout development and during the lifetime of HD patients (The Huntington's Disease Research Collaborative, 1993; Ross, 1995). Transgenic HD mice express a portion of htt-HD and develop a phenotype with many of the symptoms of HD after a period of normal development and growth (Carter et al., 1999; Cha et al., 1998; Mangiarini et al., 1996). Using differential display RT PCR, northern blot and in situ hybridization, we have demonstrated that PDE10A mRNA levels decline in the striatum of HD mice. This specific member of the PDE multigene family is highly expressed in the striatum and olfactory tubercle of mice (Soderling et al., 1999) and rats (Fujishige et al., 1999) and in the caudate and putamen of humans (Fujishige et al., 1999; Loughney et al., 1999). The levels of PDE10A were the same in 5 week old wild-type and HD mice. PDE10A mRNA levels then began to decline and were almost undetectable in the striatum and olfactory tubercle by the time the mice reached 8 weeks of age. This time coincides with the onset of overt motor symptoms in the HD mice indicating that the loss of PDE10A in striatal neurons leads to dysfunction of the nuclei that control movement. The R6/2 mice develop the HD phenotype in the absence of cell death. The decrease in PDE10A mRNA, therefore, is not due to the loss of PDE10A-expressing cells but rather a change in steady-state RNA levels that occurs due to the expression of mutant huntingtin.

The particular isoform that decreases in HD is PDE10A. PDE10A has been cloned from human lung and fetal brain cDNA libraries (Fujishige et al., 1999; Loughney et al., 1999). It appears that the presence of the expanded polyglutamine tract in huntingtin alters gene expression in the striatum, and that this is the mechanism by which only a small group of neurons in the striatum and cortex are rendered vulnerable to this ubiquitously expressed mutant protein.

### Example 8

### PDE10A is Highly Conserved Among Mammalian Species

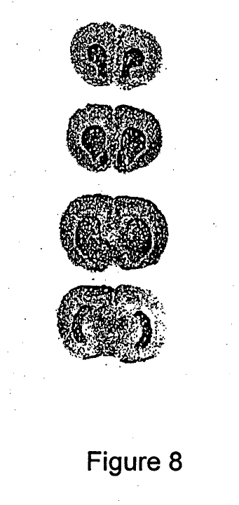

The oligonucleotide (probe 1) complementary to the coding strand of the PDE10A transcript, was also used as an in situ hybridization probe against coronal brain sections derived from adult rats. FIG. 8 shows in situ hybridization analysis of adult rat brain sections using oligonucleotide probe 1 complementary to the coding-strand of PDE10A revealed that the pattern of expression of PDE10A is the same in rats and mice. The hybridization conditions used to detect the rat homologue of PDE10A in rat brain tissue differed from those used to detect the transcript in mice only in that the stringency of the post-hybridization washes were reduced.

No hybridization was observed in the rat striatum using the post-hybridization washes employed following the in situ hybridization of mouse brain sections. However, when the stringency of the post-hybridization washes was lowered (2×60′ in 1×SSC@42° C., 2×60′ in 0.5×SSC@42° C., 2×60′ in 0.25×SSC@room temperature), the PDE10A oligonucleotide probe specifically labelled the adult rat striatum, nucleus accumbens and olfactory tubercule in a pattern indistinguishable from that observed in mouse brain sections. It appears, therefore, that a transcript which shares nucleotide sequence and expression pattern is present in both mice and rats. The evolutionary conservation of PDE10A suggests that it is important for normal function of the basal ganglia.

By northern blot, Fujishige et al. (1999) demonstrated that PDE10A is expressed in human fetal brain. The homology between mouse and human PDE10A is extremely high (data not shown).

### Example 9

### Analysis of PDE10A in Genomic DNA

Because the transgenic mice employed in this study have a copy of the human HD 5′ UTR, exon 1 with expanded CAG repeat and 262 bp of the intron 1 that has been integrated into an undefined locus of the mouse genome, it was possible that the integration event disrupted the PDE10A gene preventing its expression in the HD mouse striatum. Genomic DNA was isolated from wild-type and HD mice and subjected to Southern blot analysis.

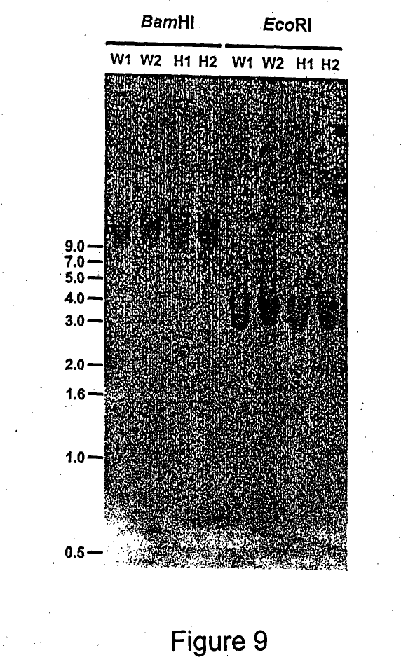

Genomic DNA was isolated from wild-type and HD mice and subjected to Southern blot analysis using pPDE10A as a hybridization probe. The size of the BamHI and EcoRI fragments that are present in the transgenic R6/2 line that correspond to the insertion of the human exon 1 gene fragment are 1.9 and 0.8 (BamHI) and 1.9 (EcoRI) kb. Analysis of the size of the fragments that hybridized with pPDE10A demonstrated that there was no difference in the size of the hybridizing fragments between the wild-type and HD mice. FIG. 9 shows the genomic DNA restriction fragments that hybridized with pPDE10A were the same in wild-type and HD mice. The size of the hybridizing BamHI and EcoRI fragments in each genomic DNA sample is approximately 8 kb and 3 kb, respectively. If the 1.9 kb SacI-EcoRI HD gene fragment integrated into the genome within the BamHI and EcoRI fragments that hybridized with the DHDM cDNA cloned insert, the sizes of the HD hybridizing bands would have been distinct from those of the wild-type. This Southern blot analysis indicates that the gene encoding PDE10A is present as a single-copy in the mouse genome. The numbers at the left of the blot are the relative mobility of molecular weight markers (1 kb ladder, BioRad).

The PDE10A cDNA has since been cloned using a bioinformatics search strategy involving screening of the expressed sequence tag (EST) database for novel PDE cDNA clones. Independently, the mouse PDE10A cDNA was identified after an EST search for novel PDEs with conserved cGMP binding domains (Soderling et al., 1999). The rat isoforms of PDE10A and splice variants have also been described (Fujishige et al., 1999). Human, mouse and rat PDE10A splice variants differ in their 5′ untranslated and part of the 5′ coding region but are identical in the coding region when the various splice variants are compared within each species. The human, mouse and rat PDE10A coding regions contain 779, 779 and 794 amino acids, respectively, encoding a protein of approximately 88.5 kDa.

### Example 10

### Distribution of PDE10A

In mouse, PDE10A mRNA was detected in testis and to a much lesser extent in brain but not in heart, spleen, lung, liver, skeletal muscle, kidney, ovary, pancreas, smooth muscle, eye or in total RNA isolated from 7, 11, 15 or 17 day old embryo (Soderling et al., 1999). This data agrees with the PDE10A mRNA pattern of distribution that we observed in wild-type and pre-symptomatic HD mice. In mice, two different size transcripts are detected in northern blots using the coding region as a probe. In mouse testis, the most abundant transcript is approximately 4 kb. A 9.5 kb transcript was also detected in mouse testis. It appears that the most abundant transcript in mouse brain is 9.5 k. Similarly, two sized PDE10A transcripts were observed in rats, however, it appears that, in rat, the 4 kb mRNA is expressed exclusively in testis while the 9.5 kb mRNA is expressed exclusively in brain (Fujishige et al., 1999). Within the brain, the rat PDE10A mRNA was expressed in striatum and olfactory tubercle and not cortex, cerebellum, hippocampus, midbrain or brainstem. In humans, PDE10A is expressed in the caudate, putamen and testis. As was observed in rodents, mRNAs of approximately 4 and 10 kb hybridized with the PDE10A probe. Again, it appears that, although both sized transcripts are present in brain and testis, the larger mRNA is predominant in the caudate and putamen and the smaller sized transcript is present in the testis. Each of the mouse, rat and human PDE10A sequences are not longer than 4 kb and span the coding region and parts of the 3′ UTR. The difference in abundance of the short and long transcript in the testis and brain, respectively, in all three species suggest that the 3′ UTR functions to provide transcript stability in the brain. As such, we present the complete sequence of the brain-specific transcript of PDE10A derived from mouse.

### Example 11

### Modulating Activity of PDE10A Using cGMP-PDE Activity

Cyclic nucleotides are the predominant second messengers that activate cellular signaling pathways (Beavo, 1995; Conti and Jin, 1999). The concentration of intracellular cyclic nucleotides is dependent on their rate of synthesis by adenyl and guanyl synthase, the rate of efflux from the cell, and the rate of degradation. PDEs hydrolyze cAMP and cGMP limiting both the duration and amplitude of the cyclic nucleotide signal (Beavo, 1995; Conti and Jin, 1999). In mammals, PDEs are encoded by a large multigene family. The various PDE family members have tissue-specific patterns of expression (Conti and Jin, 1999). PDEs have also been described in Caenorhabditis, Drosophila, Dictyostelium, Saccharomyces, Candida and Vibrio species demonstrating that this enzyme has been conserved throughout evolution. In mammals, PDEs are encoded by at least 10 gene families, each composed of one or more genes. In addition, numerous splice variants of individual gene family members have been described. These splice variants alter the 5′ domain of the protein but share identical nucleotide binding and catalytic domains. The catalytic domain, found in the carboxy-terminus of the enzyme, is ˜275 amino acids and highly conserved in amino acid sequence in all PDEs. In total, it appears that there are ˜50 PDEs expressed within the mammalian body. Some PDEs are expressed in multiple tissues while others have a very limited tissue-specific distribution (Conti and Jin, 1999).

PDE gene families differ with respect to their affinity for cAMP and cGMP and their dependence on calcium and calmodulin (Beavo, 1995). Moreover, some PDEs are inhibited or activated by binding cyclic nucleotides to a non-hydrolytic site. For example, PDE2A has a lower Kₘ for cGMP than cAMP although it hydrolysed both nucleotides. The binding of cGMP to an allosteric activator site within PDE2 enhances the rate of catalysis of cAMP. PDE2 is, therefore, a cGMP-stimulated cGMP and cAMP phosphodiesterase (Beavo, 1995). Conversely, the affinity of PDE4 for cAMP is much greater than for cGMP and PDE4 activity is not affected by cGMP or calmodulin (Beavo, 1995). The differences in substrate preference, modulation of activity and tissue-specific patterns of expression suggest that subtle alterations in the relative levels of cAMP and cGMP mediated through the action of various PDEs lead to a wide range of responses to extracellular signals.

cGMP-PDE activity of compounds is measured using a one-step assay adapted from Wells at al. (Wells, J. N., Baird, C. E., Wu, Y. J. and Hardman, J. G., Biochim. Biophys. Acta 384:430 (1975)) and adopted by Beavo et al, U.S. Pat. No. 6,037,119. The reaction medium contains 50 mM Tris-HCl, pH 7.5, 5 mM Mg-acetate, 250 μg/mL 5′-Nucleotidase, 1 mM EGTA and 0.15 μM 8-[³H]-cGMP. The enzyme used is a human recombinant PDE V (ICOS, Seattle U.S.A.).

Compounds of interest are dissolved in DMSO finally present at 2% in the assay. The incubation time was 30 minutes during which the total substrate conversion did not exceed 30%.

The IC₅₀ values for the compounds examined are determined from concentration-response curves using typically concentrations ranging from 10 nM to 10 μM. Tests against other PDE enzymes using standard methodology also show compounds highly selective for the cGMP specific PDE enzyme.

Rat aortic smooth muscle cells (RSMC) are prepared according to Chamley et al. in Cell Tissue Res. 177:503-522 (1977) and used between the 10th and 25th passage at confluence in 24-well culture dishes. Culture media is aspirated and replaced with PBS (0.5 mL) containing the compound tested at the appropriate concentration. After 30 minutes at 37° C., particulates guanylate cyclase are stimulated by addition of ANF (100 nM) for 10 minutes. At the end of incubation, the medium is withdrawn and two extractions were performed by addition of 65% ethanol (0.25 mL). The two ethanolic extracts are pooled and evaporated until dryness, using a Speed-vat system. c-GMP was measured after acetylation by scintillation proximity immunoassay (AMERSHAM). The EC₅₀ values are expressed as the dose giving half of the stimulation at saturating concentrations.

### Example 12

### Selected Modulators of PDE10A Activity

The catalytic domain of PDE10A is most similar in amino acid sequence to PDE5A, PDE2A, PDE6B and PDE6A. These members of the PDE family each contain a cGMP binding sequence that is not observed in other PDE family members. The non-catalytic cGMP binding sites (GAF) domains found in PDE2, 5 and 6 are also found in PDE10. At least for PDE2, this site acts as an allosteric activator for cAMP hydrolytic activity. The GAF domain of PDE10A binds other small molecules that act as allosteric activators. PDE10A is a cAMP and cAMP-inhibited cGMP PDE (Fujishige et al., 1999; Fujishige et al., 1999; Loughney et al., 1999; Soderling et al., 1999).

Attenuation of the production of cAMP, may ameliorate the symptoms of HD and positively affect gene expression. Pharmaceutically acceptable modulators of cAMP include quinpirole, alloxan, miconazole nitrate, MDL-12330A, and tetracyline derivatives such as demeclocycline and minocycline.

Compounds which are potent and selective modulators of cGMP-specific PDE, and are useful in a variety of therapeutic areas are taught by Daugan et al, U.S. Pat. No. 5,981,527, PCT publication No. WO 00/15639 to Icos Corporation and PCT publication No. WO 00/15228 to Icos Corporation, which are incorporated herein by reference. Such compounds include, for example:

(6R,12aR)-2,3,6,7,12,12a-Hexahydro-6-(5-benzofuranyl)-2-methyl-pyrazino[2′,1′:6,1]pyrido[3,4-b]indole-1,4-dione,

(6R,12aR)-2,3,6,7,12,12a-Hexahydro-6-(5-benzofuranyl)-pyrazino[2′,1′:6,1]py rido[3,4-]indole-1,4-dione,

(6R,12aR)-2,3,6,7,12,12a-Hexahydro-6-(5-benzofuranyl)-2-isopropyl-pyrazino[2′,1′:6,1]pyrido[3,4-b]indole-1,4-dione,

(3S ,6R,12aR)-2,3,6,7,12,12a-Hexahydro-6-(5-benzofuranyl)-3-methyl-pyrazino[2′,1′:6,1]pyrido[3,4-b]indole-1,4-dione, and

(3S,6R,12aR)-2,3,6,7,12,12a-Hexahydro-6-(5-benzofuranyl)-2,3-dimethyl-pyraz ino[2′,1′:6,1]pyrido[3,4-b]indole-1,4-dione.

PDE1B1 is expressed throughout the brain and is most abundant in the striatum, nucleus accumbens and olfactory tubercle (Polli and Kincaid, 1994; Yan et al., 1994). PDE1B is a cGMP, Ca/calmodulin-dependent PDE. Therefore, PDE1B and 10A are both expressed in the majority, but not all, striatal neurons and, it is likely that both genes are co-expressed in a subset of striatal projection neurons. Selective inhibitors for PDE1 include KS-505, IC224, and SCH 51866. Of these inhibitors, it appears that SCH 51866 has a ten-fold higher Km for PDE1 than PDE10 (Soderling et al., 1999). The non-specific PDE inhibitor IBMX is a potent inhibitor of PDE10A. Dipyridamole and SCH51866 had the highest potency of inhibitors tested on PDE10A activity. Dipyridamole was considered to be a PDE5- and PDE6-specific inhibitor, however, the Km for dipyridamole is 10 times higher for PDE10A than the other PDEs (Soderling et al., 1999). Selective inhibitors of PDE5, 2, 3 and 4 had much greater IC50 for PDE10 (Soderling et al., 1999).

### Example 13

### Clinical Use of PDE10A Modulator

A 38 year-old female was admitted to hospital from a long-term care facility due to progressive deterioration of her physical and mental symptoms caused by Huntington's disease. The patient had been diagnosed with Huntington's disease at age 26. Prior to admission to the hospital, she had become increasingly aggressive and uncooperative. Moreover, there appeared to be an increase in the number of psychotic episodes. SPECT showed no abnormality of brain blood flow but MRI showed bilateral caudate atrophy as well as global atrophy of the cerebrum and corpus callosum.

The patient had been stable for a number of years on the antipsycotic haloperidol (3 mg/day). For the last two years, the haloperidol had been replaced by olanzapine (2.5-7.5 mg/day).

Minocycline, a tetracycline derivative, was administered at 50 mg twice daily for 7 days, followed by 100 mg twice daily for 7 days and finally 200 mg twice daily for 5 weeks. After 5 weeks of 200 mg twice daily minocycline administration, there was a mild improvement compared to the baseline clinical global assessment made at the time of admission. The minocycline treatment was suspended for 7 days. Due to a significant increase in the number of aggressive incidence and decrease in cooperativity, minocycline (200 mg twice daily) treatment was resumed. The patient responded within 3 days to the resumed minocycline-treatment with a return to mild-improvement compared to the baseline clinical global assessment made at the time of admission. Minocycline (200 mg twice daily) treatment will continue indefinitely. The improvement in behaviour and decrease in apparent psychosis has allowed for the transfer of the patient from the acute care facility back to long-term care.

While the present invention has been described in terms of specific embodiments, it is understood that variations and modifications will occur to those skilled in the art. Accordingly, only such limitations as appear in the appended claims should be placed on the invention.

Non-Patent Literature Cited

Atschul, S. F. et al. (1990), J. Molec. Biol. 215: 403.

Babity, J. M., Armstrong, J. N., Plumier, J.-C., Currie, R. W., and Robertson, H. A. (1997a) A novel seizure-induced synaptotagmin gene identified by differential display. Proc. Nat. Acad. Sci. U.S.A. 94: 2638-2641.

Babity, J. M., Newton, R. N., Guido, M. E. and Robertson, H. A. (1997b) The Application Of Differential Display To The Brain: Adaptations For The Study Of Heterogeneous Tissue. Methods Mol Biol, 85 (1997) 285-95.

Barney (1991) in PCR Methods and Applications, August, Vol. 1(1), page 5.

Beavo, J. (1995). Cyclic nucleotide phosphodiesterase: functional implications of multiple isoforms. Physiol. Rev. 75, 725-748

Beretta, S., Robertson, H. and Graybiel, A. (1992) Dopamine and glutamate agonists stimulate neuron-specific expression of Fos-like protein in the striatum. J. Neurophysiol. 68, 767-777

Bennett, D. et al., (1995) Journal of Molecular Recognition, Vol. 8 52-58.

Berke, J. D., Paletzki, R. F., Aronson, G. J., Hyman, S. E. and Gerfen, C. R. (1998) A Complex Program of Striatal Gene Expression Induced by Dopaminergic Stimulation. J. Neurosci. 18: 5301-5310.

Caine E. D., Hunt R. D., Weingartner H., Ebert M. H. (1978) Huntington's dementia. Clinical and neuropsychological features. Arch-Gen-Psychiatry. 35, 377-84.

Carillo, H., and Lipman, D. (1988), SIAM J. Applied Math., 48: 1073.

Carter, R. J., Lione, L. A., Humby, T., Mangiarini, L., Mahal, A., Bates, G. P., Dunnet, S. B., and Morton, A. J. (April 1999). Characterization of progressive motor deficits in mice transgenic for the human Huntington's diease mutation. J Neurosci 19, 3248-57

Cha, J.-H. J., Kosinski, C. M., Kerner, J. A., Alsdorf, S. A., Mangiarini, L., Davies, S. W., Penny, John B., Bates, G. P., Young, A. B. (1998) Altered brain neurotransmitter receptors in transgenic mice expressing a portion of an abnormal human Huntington disease gene. Proc. Natl. Acad. Sci. USA 95: 6480-6485.

Conolly, B. A. (1987) Nucleic Acids Res. 15:15(7): 3131.

Conti, M., and Jin, S. L. (1999). The molecular biology of cyclic nucleotide phosphodiesterases. Prog Nucleic Acid Res Mol Biol 63, 1-38

Crino, P., Khodakhah, K., Becker, K., Ginsberg, S., Hemby, S. and Eberwine, J. (1998) Presence and phosphorylation of transcription factors in developing dendrites. Proc. Natl. Acad. Sci. USA 95: 2313-2318

Davies, S. W., Turmaine, M., Cozens, B. A., DiFiglia, M., Sharp, A. H., Ross, C. A., Scherzinger, E., Wanker, E. E., Mangiarini, L. and Bates, G. P. (1997) Formation of neuronal intranuclear inclusions underlies the neurological dysfunction in mice transgenic for the HD mutation. Cell 90, 537-548.

Davis et al., (1986) Basic Methods in Molecular Biology.

Denovan-Wright, E. M., Newton, R. A., Armstrong, J. N., Babity, J. M., Robertson, H. A. (1998) Acute administration of cocaine, but not amphetamine, increases the level of synaptotagmin IV mRNA in the dorsal striatum of rat. Mol. Brain Res. 55, 350-354.

Denovan-Wright, E. M., Gilby, K. L., Howlett, S. E. and Robertson, H. A. (1999) Cloning of differentially expressed brain cDNA. In Differential Display—A Practical Approach, H. A. Robertson and R. A. Leslie, Eds. Oxford University Press (in press).

Devereux, J., et al. (1984), Nucleic Acids Research 12(1): 387.

Douglass, J., McKinzie, A. A. and Couceyro, P. (1995) PCR differential display identifies a rat brain mRNA that is transcriptionally regulated by cocaine and amphetamine. J. Neurosci. 15, 2471-2481.

Douglas, J. and Daoud, S. (1996) Characterization of the human cDNA and genomic DNA encoding CART: a cocaine- and amphetamine-regulated transcript. Gene 169, 241-245

Eberwine, J. (1996) Amplification of mRNA populations using aRNA generated from immoblized oligo(dt)-T7 primed cDNA. Biotechniques 20: 584-589

Eberwine, J., Yeh, H., Miyashiro, K., Cao, Y., Nair, S., Finnell, R., Zettel, M., Coleman, P., (1992) Analysis of gene expression in single live neurons. Proc. Natl. Acad. Sci. USA 89: 3010-3014

Fujishige, K., Kotera, J., Michibata, H., Yuasa, K., Takebayashi, S., Okumura, K., and Omori, K. (Jun. 25, 1999). Cloning and characterization of a novel human phosphodiesterase that hydrolyzes both cAMP and cGMP (PDE10A). J Biol Chem 274, 18438-45

Glass, M., Faull, R. L. M. and Dragunow, M. (1993) Loss of cannabinoid receptors in the substantia nigra in Huntington's disease. Neuroscience 56: 523-527

Good et al (1977), Nucl. Acid Res 4:2157.

Gribskov, M. and Devereux, J., eds., Sequence Analysis Primer, M Stockton Press, New York, 1991.

Griffin, A. M., and Griffin, H. G., eds., Computer Analysis of Sequence Data, Part I, Humana Press, New Jersey, 1994.

Group THDCR (1993) A novel gene containing a trinucleotide repeat that is extended and unstable on Huntington's disease chromosomes. Cell 72: 971-983.

Hardy, J. and Gwinn-Hardy, K. (1998) Genetic classification of primary neurodegeneraive disease. Science 282: 1075-1079

Hinnen et al., (1978) PNAS USA 75:1929-1933.

Houbenweyl, (1987) Methods of Organic Chemistry, ed. E. Wansch, Vol. 15 I and II, Thieme, Stuttgart.

Innis, M. A. and D. H. Gelfand (1989), PCR Protocols, A guide to Methods and Applications, M. A. Innis, D. H. Gelfand, J. J. Shinsky and T. J. White eds, pp 3-12, Academic Press.

Johanson, K. et al. (1995), The Journal of Biological Chemistry, Vol. 270, No. 16, pp 9459-9471.

Klement, I. A., Skinner, P. J., Kaytor, M. D., Yi, H., Hersch, S. M., Clark, H. B., Zoghbi, H. Y. and Orr, H. T. (1998) Ataxin-1 nuclear localization and aggregation: role in polyglutamine-induced disease in SCA1 transgenic mice. Cell 95: 41-53.

Lesk, A. M., ed., Computational Molecular Biology, Oxford University Press, New York, 1988.

Liang, P. and Pardee, A. B. (1992) Differential display of eukaryotic messenger RNA by means of the polymerase chain reaction. Science 257: 967-971.

Livesey, F. J., O'Brien, J. A., Li, M., Smith, A. G., Murphy, L. J. and Hunt, S. P. (1997) A Schwann cell mitogen accompanying regeneration of motor neurons. Nature 390: 614-618.

Livesey, F. J. and Hunt, S. P. (1996) Identifying changes in gene expression in the nervous system: mRNA differential display. Trends Neurosci. 19: 84-88

Loughney, K., Snyder, P. B., Uher, L., Rosman, G. J., Ferguson, K., and Florio, V. A. (June 1999). Isolation and characterization of PDE10A, a novel human 3′, 5′-cyclic nucleotide phosphodiesterase. Gene 234, 109-17

Ludlow, C. L., Connor, N. P., Bassich, C. J. (1987) Speech timing in Parkinson's and Huntington's disease. Brain-Lang, 32, 195-214.

Mangiarini, L., Sathasivam, K., Seller, M., Cozens, B., Harper, A., Hetherington, C. Lawton, M., Trottier, Y., Lehrach, H., Davies, S. W. and Bates, G. P. (1996) Exon 1 of the HD gene with an expanded CAG repeat is sufficient to cause a progressive neurological phenotype in transgenic mice. Cell 87: 493-506.

Merrifield, (1964), J. Am. Chem. Assoc. 85:2149-2154.

Okano, J., (1988) Neurochem. 56: 560.

Pardee, A. B. (1997) Complete genome expression monitoring: the human race. Nat. Biotechnol. 15: 1343-1344

Polli, J. W., and Kincaid, R. L. (1994). Expression of a calmodulin-dependent phosphodiesterase isoform (PDE1B1) correlates with brain regions having extensive dopaminergic innervation. J. Neurosci. 14, 1251-1261

Qu,-D., Ludwig,-D. S., Gammeltoft, S., Piper, M., Pelleymounter, M. A., Cullen, M. J. Mathes, W. F., Przypek, R., Kanarek, R. and Maratos-Flier, E. (1996) A role for melanin-concentrating hormone in the central regulation of feeding behaviour. Nature 380: 243-247.

Richfield, E. K. and Herkenham, M. (1994) Selective vulnerability in Huntington's disease: preferenctial loss of cannabinoid receptors in lateral globus pallidus. Ann. Neurol. 36: 577-584.

Ross, C. A., (1997) Intranuclear neuronal inclusions: a common pathogenic mechanism for glutamine-repeat neurodegenerative diseases? Neuron 19: 1147-1150.

Saiki et al., (1986) Nature, 324: 163-166.

Sambrook, et al, (1989), Molecular Cloning, A Laboratory Manual, Cold Spring Harbor Laboratory Press, Cold Spring Harbor, N.Y.

Saudou, F., Finkbeiner, S., Devys, D. and Greenberg, M. E. (1998) Huntingtin acts in the nucleus to induce apoptosis but death does not correlate with the formation of intranuclear inclusions. Cell 95: 55-66.

Sisodia, S. S., (1998) Nuclear inclusions in glutamine repeat disorders: are they pernicious, coincidental or beneficial? Cell 95: 1-4.

Smith, D. W., ed., Biocomputing: Informatics and Genome Projects, Academic Press, New York, 1993.

Soderling, S. H., Bayuga, S. J., and Beavo. J. A. (June 1999). Isolation and characterization of a dual-substrate phosphodiesterase gene family: PDE10A. Proc Natl Acad Sci U S A 96, 7071-6

von Heinje, G., Sequence Analysis in Molecular Biology, Academic Press, 1987.

Yan, C., Bentley, J. K. Sonnenburg, W. K., and Beavo, J. A. (1994). Differential expression of the 61 kDa a 63 kDa calmodulin-dependent phosphodiesterase in the mouse brain. J. Neurosci. 14, 973-984

Young, A. B., Penney, J. B., Starosta-Rubinstein, S., Markel, D. S., Berent, S., Giordani, B., Ehrenkaufer, R., Jewett, D., Hichwa, R. (1986) PET scan investigations of Huntington's disease: cerebral metabolic correlates of neurological features and functional decline. Ann-Neurol. 20, 296-303.

What is claimed is:

1. A method for identifying a modulator of PDE10 phosphodiesterase activity, the method comprising:
(i) obtaining a purified PDE10 having phosphodiesterase activity;
(ii) incubating the purified PDE10 with a cyclic nucleotide in the presence of a candidate molecule;
(iii) identifying whether the candidate molecule is a modulator of PDE10 by quantitatively measuring the phosphodiesterase activity of the PDE10 in the presence of the candidate molecule and comparing the activity with PDE10 phosphodiesterase activity in the absence of the candidate molecule.

2. A method for identifying a modulator of PDE10 phosphodiesterase activity, the method comprising:
(i) obtaining a purified PDE10 and at least one additional phosphodiesterase with phosphodiesterase activity;
(ii) incubating the purified PDE10 with a cyclic nucleotide in the presence of a candidate molecule and quantitatively measuring the PDE10 phosphodiesterase activity;
(iii) comparing the PDE10 activity in the presence of the candidate molecule with the activity in the absence of the candidate molecule
(iv) incubating the at least one additional phosphodiesterase with a cyclic nucleotide in the presence of the candidate molecule and quantitatively measuring the at least one additional phosphodiesterase activity;
(v) comparing the at least one additional phosphodiesterase activity in the presence of the candidate molecule with the activity in the absence of the candidate molecule;
(vi) identifying whether the candidate molecule is a modulator with specificity by comparing modulation by the candidate molecule of the of PDE10 activity and of the at least one additional phosphodiesterase activity.

3. A method for identifying a specific modulator of PDE10 according to claim 1, the method further comprising:
(i) quantitatively measuring at least one additional phosphodiesterase activity in the presence and absence of the candidate molecule; and
(ii) identifying a specific modulator of PDE10 as one which preferentially modulates the activity of PDE10 compared to the activity of at least one other phosphodiesterase.

4. A method according to claim 1 or 2, wherein the purified PDE10 is purified mammalian PDE10A.

5. A method according to claim 4 wherein the purified PDE10A is purified human PDE10A.

6. A method according to any of claims 1-3, wherein the purified PDE10 is obtained by expression of recombinant DNA.

7. A method according to claim 6, wherein the recombinant DNA is cDNA.

8. A method according to claim 6, wherein the recombinant DNA is genomic DNA.

9. A method according to claim 6, wherein the recombinant DNA is expressed in cells.

10. A method according to claim 9, wherein the cells are of mammalian, yeast or bacterial origin.

11. A method according to claim 9 wherein the recombinant DNA is expressed in cells previously subjected to DNA transfection.

12. A method according to claim 10, wherein the cells are lysed before obtaining the purified PDE10.

13. A method for identifying a modulator of PDE10 according to claim 1 or 2, wherein the cyclic nucleotide is colorimetrically labeled and wherein step (i) further comprises quantitatively measuring a colorimetric change in the presence and absence of a candidate molecule for determining PDE10 activity.

14. A method for identifying a modulator of PDE10 according to claim 1 or 2, wherein the cyclic nucleotide is fluorescently labeled and wherein step (i) further comprises quantitatively measuring a change in fluorescent signal in the presence and absence of a candidate molecule for determining PDE10 activity.

15. A method for identifying a modulator of PDE10 according to claim 1 or 2, by use of a marker responsive to changes in PDE10 activity in the presence and absence of a candidate molecule.

16. A method for identifying a modulator of PDE10 according to claim 1 or 2, wherein the cyclic nucleotide is radiolabeled and wherein step (i) further comprises quantitatively measuring a change in radioactive signal in the presence and absence of a candidate molecule for determining PDE10 activity.

17. A method for identifying a modulator of PDE10 according to claim 1 or 2, wherein the cyclic nucleotide is radiolabeled cAMP or cGMP and wherein step (i) further comprises quantitatively measuring a change in 8-[³H]-cGMP or 8-[³H]-cAMP hydrolysis in the presence and absence of a candidate molecule for determining PDE10 activity by comparing the radioactive signal of non-hydrolyzed 8-[³H]-cGMP or 8-[³H]-cAMP nucleotides to the radioactive signal of the corresponding hydrolyzed nucleosides in the presence and absence of a candidate molecule.

18. A method for identifying a modulator of PDE10 according to claim 1 or 2, wherein the modulator is an inhibitor of PDE10 activity.

19. A method for identifying a modulator of PDE10 according to claim 1 or 2, wherein the modulator is an enhancer of PDE10 activity.

20. A method according to claim 1, wherein the modulator acts as a therapeutic agent for a triplet repeat disorder.

21. A method according to claim 1, wherein the modulator acts as a therapeutic agent suitable for treating at least one of Alzheimer's disease, Parkinson's disease, schizophrenia, depression, anxiety, stress, trauma, and stroke.

22. A method according to claim 1, wherein the candidate molecule is selected from the group consisting of a small organic molecule, a peptide and an antibody.

23. A method for identifying a modulator of PDE10 using a population of cells, the method comprising:
(i) providing a population of cells and, optionally, a source of PDE10;
(ii) quantitatively measuring PDE10 activity using the population of cells to establish a first PDE10 activity profile in the absence of a candidate molecule and a second PDE10 activity profile in the presence of a candidate molecule;
(iii) identifying a modulator of PDE10 using the population of cells as one which effects a change in PDE10 activity in the second PDE10 activity profile relative to the first PDE10 activity profile.

24. A method according to claim 23 wherein the PDE10 activity is PDE10A activity.

25. A method according to claim 23 or 24, wherein the modulator acts as a therapeutic agent for a triplet repeat disorder.

26. A method according to claim 23 or 24, wherein the modulator acts as a therapeutic agent suitable for treating at least one of Alzheimer's disease, Parkinson's disease, schizophrenia, depression, anxiety, stress, trauma, and stroke.

27. A method according to claim 23 or 24, wherein the candidate molecule is selected from the group consisting of a small organic molecule, a peptide and an antibody.

28. A method for identifying a modulator of PDE10 according to claim 23 wherein the method further comprises lysing the cells before quantitatively measuring PDE10 activity.

29. A method for identifying a modulator of PDE10 according to claim 23 or 28, wherein the cyclic nucleotide is colorimetrically labeled and wherein step (i) further comprises quantitatively measuring a colorimetric change in the presence and absence of a candidate molecule for determining PDE10 activity.

30. A method for identifying a modulator of PDE10 according to claim 23 or 28, wherein the cyclic nucleotide is fluorescently labeled and wherein step (i) further comprises quantitatively measuring a change in fluorescent signal in the presence and absence of a candidate molecule for determining PDE10 activity.

31. A method for identifying a modulator of PDE10 according to claim 23 or 28, by use of a marker responsive to changes in PDE10 activity in the presence and absence of a candidate molecule.

32. A method for identifying a modulator of PDE10 according to claim 23 or 28, wherein the cyclic nucleotide is radiolabeled and wherein step (i) further comprises quantitatively measuring a change in radioactive signal in the presence and absence of a candidate molecule for determining PDE10 activity.

33. A method for identifying a modulator of PDE10 according to claim 23 or 28, wherein the cyclic nucleotide is radiolabeled cAMP or cGMP and wherein step (i) further comprises quantitatively measuring a change in 8-[³H]-cGMP or 8-[³H]-cAMP hydrolysis in the presence and absence of a candidate molecule for determining PDE10 activity by comparing the radioactive signal of non-hydrolyzed 8-[³H]-cGMP or 8-[³H]-cAMP nucleotides to the radioactive signal of the corresponding hydrolyzed nucleosides in the presence and absence of a candidate molecule.

34. A method according to claims 23 or 24, wherein quantitatively measuring PDE10 activity further comprises visualizing the population of cells after incubation with a candidate molecule and measuring changes in the population's physical properties in comparison to a population of cells not treated with a candidate molecule

35. A method according to claim 23, wherein the population of cells is a population of animal cells.

36. A method according to claim 35, wherein the animal cells are taken from a transgenic animal.

37. A method according to claim 35, wherein the population of cells is a population of human cells.

38. A method according to claim 36, wherein the population of cells is a population of human neuronal cells.

39. A method according to claim 35, wherein the population of cells expresses endogenous PDE10.

40. A method according to claim 36, wherein the population of cells is a population of murine cells.

41. A method according to claim 40, wherein the population of cells is a population of murine neuronal cells.

42. A method according to claim 41, wherein the population of cells a population of murine striatum cells.

43. A method according to claim 23, wherein the identified PDE10 modulator is an enhancer of PDE10 activity.

44. A method according to claim 23, wherein the identified PDE10 modulator is an inhibitor of PDE10 activity.

45. A method for treating an animal subject to a disorder of the basal ganglia with a modulator of a PDE10 the method comprising:
(i) providing an animal having a disorder of the basal ganglia; and
(ii) treating the animal with an effective amount of a PDE10 modulator in a desired formulation for a prescribed time period such that treatment alleviates symptoms of the disease and/or delays manifestation of the basal ganglia disorder.

46. A method for treating an animal according to claim 45 wherein the animal is a human.

47. A method according to claim 45 wherein the disorder of the basal ganglia includes any disorder characterized by a gene defect containing an expanded tract of CAG repeats or expression of gene products containing polyglutamine tracts.

48. A method according to claim 47 wherein the disorder of the basal ganglia is Huntington's disease, Alzheimer's disease, Parkinson's disease, schizophrenia, depression, anxiety, stress, trauma, or stroke.

49. A pharmaceutical formulation comprising:
an effective amount of a modulator of PDE10 activity in an acceptable carrier.

50. A pharmaceutical formulation according to claim 49, further comprising at least one of an emulsifier, a buffer, a glidant, a lubricant, an anti-oxidant, a reducing agent, a colorant, a preservative, a flavoring agent, a filler, or any other appropriate excipient.

51. A pharmaceutical formulation according to claim 49 wherein the modulator of PDE10 activity is formulated for delivery including oral, intravenous, subcutaneous, intraperitoneal, intramuscular, brain infusion, brain implantation, transdermal, transmucosal, sustained-release implantation, or any other delivery that is effective for delivery of the modulator of PDE10 activity.

52. A pharmaceutical formulation according to claim 49 wherein the acceptable carrier comprises a buffer, a saline solution, dextrose, water, glycerol, ethanol, oil, or combinations thereof.

53. A method for assessing PDE10 activity in an animal model, such animal model being suitable for use in the testing of a disorder of the basal ganglia, the method comprising:
(i) providing an animal model of a disorder of the basal ganglia;
(ii) treating the animal model with modulator of PDE10 activity;
(iii) assessing PDE10 activity in the animal model wherein activity is assessed by monitoring at least one of the following relative to a placebo-treated animal and/or a non-treated animal:
(a) a delay in onset of progression of symptoms;
(b) a reversal in manifestation of symptoms;
(c) a lessening of symptoms;
(d) a characteristic change in a disease marker consisting of: a histopathological marker, a biochemical marker including an electophysiological marker, synaptic remodeling or a change in neuronal function; or a nuleic acid marker including a change in RNA or protein composition, structure, function, and expression;
wherein such change is indicative of disease progression.

54. A method according to claim 53 wherein the animal model is a model of a disorder of the basal ganglia characterized by a gene defect containing an expanded tract of CAG repeats.

55. A method according to claim 53 wherein the animal model is a model of Huntington's disease, Alzheimer's disease, Parkinson's disease, schizophrenia, depression, anxiety, stress, trauma, or stroke.

56. The method of claim 53 or claim 54 wherein the animal is a transgenic animal.

57. The method of claim 53 wherein the modulator of PDE acts by inhibiting PDE10 gene expression.

58. The method of claim 57 wherein the inhibitor of PDE10 gene expression is antisense RNA or a ribozyme.

59. The method of claim 53 wherein PDE10 consists of PDE10A.

60. The method of claim 53 wherein the PDE10 modulator is an enhancer or an activator of PDE10 activity.

61. The method of claim 53 wherein the PDE10 modulator is an inhibitor of PDE10 activity.


SEQUENCE LISTING (12 sequences)

SEQ ID NO: 1 - DNA, 3236 bp, mouse
cactgaagctggtccacgtctataaacaggtgacactggctgcagcaaaaagccattcgatccacacaaattgatcttctatcatcttggaatctgaattgcagggaggagcagtatgtaagacgaccgtttaattcaggcattccgaaggcatgagcgcatggattctgtcaccaagcgtataaaaggaccctggcattgggaaacctatgacggactgtttttgctgtagaagtagggattttacagaagtctccttgaatttgccctgcctggggcagttttgcagaggaacctgccagagatttattggctggtcagtctcttgtgaaatagtatcatgtgagaaacagtttgtagaaaaaaactatacctgggaagacctttgcaacattgttccttccatgggccaagactcagttaggaggcataaatctgcccggaataaactaggccaggatacagccatgtttagttaataatttggttttagaattcacacaggcaggattggtttttttgtgtcttggcaagtggagcatatttaacatacaggcatgggaatcctgcctcttagcttttcccaccctcttgtctcaccaagttttttctctccaaaggtttccaggaatttctcattaatggctgatgcaaacttagtgaataataatgaatataaacaatgctcacctcaccaaaattatattatttgcagtcatttgtgataacacaaattttatcgcaatggttattatttaatttgtggccacacactgtggttatcttttgttgtggttgtttctgagaaaatgttcttggatatgtaagtgccaataccagtgtgaagtattgatcccgggcagcaaaatacagcctaaggtttgtaaacatcaattctatctcagttcatcagagggcctgagaagctgcggggcagtgtaaagtaaagtatgctgggctggtggtggtcagcctcccgcctgaagagtgaccagtgctggcccgacggatcgctgagatattctcccataatggcaaaaaaataggcagtttgatgtgacctgtttagtgtggctctcctcttttgagcatgtgttagcatttttattttatactcatccagtgaactctgctcttccaagtgtgttcatgtatgtgctagatatattagcacagcctgccttctgctgcacaacgccttagagacccggcctttcaatgagcttagcttgtgctctgtttctgctctcttaggtctaaactatggtgtcagttttaatagaacaaaagtatgcatcttgccttggcttgagccttttcgttttcaatgctgacttctcccctttctctcctgtgctcaccttacctttccagagtgtaagggacaacttttaaggaggcgtgtccctggtaggggcatccctgttcaccaggtgcctgtcatcaccccacttgactgacatctaccctggtgactatgggttcctcttgtttgtagggaacggtggctccaggtggaggcatcaatctgttgggttctggttcccggctgcctttggttttgaaagtctcttctctgtatattcctaccctgcatttgctttgtgtggtgctgatgctgtgcgcagtaggattcttggatgactctccatcagtcacagactccccctgttgcaaagtgtcaggctgactcgacagtcaccgtaaaatctgagtcagtcacacacaggctgtcagccacggcttccacttgcatggctattctattttcacacgtgagtttctgttgctggctggctgactggcattatctatgctaagttgaaatcaggagtgcccagcagagcccatcattctcactgtctttgaaacaaagctgtacggtttgatcgatgaacgtatttaaagcatttcatgcaatgacaaagtgctcagtagtggaaggcaggctgtgaccagtctgcctgctccttactataattgtgaggatttgttactggaacagtacatggaggcctgaccttgtgggggcacagggtggaaccttagctgaatatagtgtgtgtctcaagaggaagtcagggtactagctcagtgctcaatctccaggtactatatatacatttgcccgttttatctctaatgtgaaataaatccccaaacacttgtttatcgtgtagcgtacctaaaagactattctattatgggtgtccccactttcttggtttggtcaccccgatcccccggtcttctgctgtatctagaacagtgactataaatgatgtatgggaatagtgtttccatatgatctgttgtctggagtatatgctacatgttcaattactgtacaaaaacccagtgcagctgatgatgcaaagcagtctctctctgtgtacagtgccccacctatttaaaaatcacgtacaascccagaacactgtgaaacacttaacataagaacaaacgcagcgtctggattctttccaaggagagcagctttctccacaggaacacagtaacaaaagaggtccgccgccatccacacccagccaagacacctcagaggccatagggacaacctccttgctggccaacacctgctggagcaggggcacaggtcccagcaactgatcctcagtggatgggtccgcagtcaaagccttaatgggctctcttttgaaggggaaagaaagaatttcaagcttatgatatccaacattattatagttgatgagttagtaaattccaaaaaaaaaagatgattttatatgtatgacataaaaaaaatctttgtaaagtgcgcaagtgcaataatttaaagaggtcttatctttgcatttataaattataaatattgtacatgtgtgtaatttttcatgtattcatttgcagtctttgtatttaaaaaaactttactgttatgtttgtataatagaacattaatcatttattataactcagacaaggtgtaaataaattcataattcaaacagccagtatatatgcatatatgggtgttacattgcaaaaatctctatctttgttctattcacatgcttaaagaagtaagaaatcttttgtggatatgtaattatacatataaagtatatatatatgtatgatacatgaaatatatttagaaatgttcataattttaatggatattctttggtgtgaataattgaatacaacatttttaaaatgaaaaaaaaaaaaaaaaaaaaaaaaaaaaaaaa

SEQ ID NO: 2 - DNA, 5752 bp, mouse
aagtgtaaataaaataaacatctaataaaaaaaattacataccatagaggaacaagataatttctgcccaacttcataccctccagcgtatagtgttgaggtttggtctgttgctgtgtattgtaatgtaatgttaaattctctacctgaaggtctaggcctacaagtgaattctcatgtttatagagttttgttgtgcaaaccttgttccttaatttaaaactatggttaaaaaacaaaacaaaactggctacagccaataactgaagggggttaccttgttgaaggggtggaaaagagagaggaggaagaagggagttcaagagaaggagaagaacaagaggagaggaggaagctgccacgaggggagatgggccatgagaacttggccaggagaaatagccagtatctggagtacaccactgaggaggtagccaggctagcagttagaagagtagattaggggttatttttcccccactccacatagttatcaaagccaaataaaataaccatagtctgagtctcatctatttgtaagctagttgggtataagattaatttggctgtactacagtttagatttctaacataggaactatcaaaaacttgctcaaacaagaacatgctgacaatattttaaaatgattatttatattgtttgcactttctaaagtttcttctaaatgttccatggtcaaattaaaaaatatacatattggctattaaattcgtctaagtggggctggagagatagctcagaggttaagagcactgactgctcttccagaggtcctgagttcaattcccagcgaccacatggtggctcacagccatctgtaatagataggatctgacgccctcttctggagtgtctgaagacagctacaatgtactcatatatattaaataaataatattagaaaattcttctaagtgtatcatttatagaatatttaatatataaagtaaatgcctcaggaaatataaacttggaattaaatcaaagaacttcatgagtagtgggccacaaaaaatgtgtaccaggggaagaccggagggaggggagaaggaagggatggagatagaattttgcctctgcattccttgggctggcacaggtataatgctgtgggaattgggaaactacaaggaagctgcaaagctgggcggaactcgtttccgcaagctgggctcatctaagtgtccatgcatggctgccacactgcagtgaactttaaaacatttgtgttccagagatgtagagatgctcacaatagtacaaaggcgggagggaggtatttccagactaagaggaagaaaaaccattgctgattaaacatctgcatatgagcgcccccacctccatacacacacacacacacacacacacacacacacaaccaaacagaacaaatacacatgcatgtctacagcctgcaggaacaaaatggtatgtctgtgaggaaccaggagatgcacaggtcctaacctctgtctcctacaagccctgaagtctggtcagggtcaaatgtacaaaagcaggctaaggaagctgtttagtgaaagatttttttcttcaactctaggaacaacctatttcctaggatttggagagtgctcaggaggaaacattcagacaactgatgctctctgtgtaccccagattcaggtattggggtagttagttgtgctcatgtatgtgctagatatattagcacagcctgccttctgctgcacaacgccttagagacccggcctttcaatgagcttagcttgtgctctgtttctgctctcttaggtctaaactatggtgtcagttttaatagaacaaaagtatgcatcttgccttggcttgagccttttcgttttcaatgctgacttctcccctttctctcctgtgctcaccttacctttccagagtgtaagggacaacttttaaggaggcgtgtccctggtaggggcatccctgttcaccaggtgcctgtcatcaccccacttgactgacatctaccctggtgactatgggttcctcttgtttgtagggaacggtggctccaggtggaggcatcaatctgttgggttctggttcccggctgcctttggttttgaaagtctcttctctgtatattcctaccctgcatttgctttgtgtggtgctgatgctgtgcgcagcaggattcttggatgactctccatcagtcacagactccccctgttgcaaagtgtcaggctgactcgacagtcaccgtaaaatctgagtcagtcacacacaggctgtcagccacggcttccacttgcatggctattctattttcacacgtgagtttctgttgctggctggctgactggcattatctatgctaagttgaaatcaggggtgcccagcagagcccatcattctcactgtctttgaaacaaagctgtacggtttgatcgatgaacgtatttaaagcatttcatgcaatgacaaagtgctcagtagtggaaggcaggctgtgaccagtctgcctgctccttactataattgtgaggatttgttactggaacagtacatggaggcctgaccttgtgggggcacagggtggaaccttagctgaatatagtgtgtgtctcaagaggaagtcagggtactagctcagtgctcaatctccaggtactatatatacatttgcccgttttatctctaatgtgaaataaatccccaaacacttgtttatcgtgtagcgtacctaaaagactattctattatgggtgtccccactttcttggtttggtcaccccgatcccccggtcttctgctgtatctagaacagtgactataaatgatgtatgggaatagtgtttccatatgatctgttgtctggagtatatgctacatgttcatttactgtacaaaaacccagtgcagctgatgatgcaaagcagtctctctctgtgtacagtgccccacctatttaaaaatcacgtacttgcccagaacactgtgaaacacttaacataagaacaaacgcagcgtctggattctttccaaggagagcagctttctccacaggaacacagtaacaaaagaggtccgccgccatccacacccagccaagacacctcagaggccatagggacaacctccttgctggccaacacctgctggagcaggggcacaggtcccagcaactgatcctcagtggatgggtctgcagccaaagccttaatgggctctcttttgaaggggaaagaaagaatttcaagcttatgatatccaatattattatagttgatgagttagtaaattccaaaaaaaaaagatgattttatatgtatgacataaaaaaaatctttgtaaagtgcgcaagtgcaataatttaaagaggtcttatctttgcatttataaattataaatattgtacatgtgtgtaatttttcatgtattcatttgcagtctttgtatttaaaaaaactttactgttatgtttgtataatagaacattaatcatttattataactcagacaaggtgtaaataaattcataattcaaacagccagtatatatgcatatatgggtgttacattgcaaaaatctctatctttgttctattcacatgcttaaagaagtaagaaatcttttgtggatatgtaattatacatataaagtatatatatatgtatgatacatgaaatatatttagaaatgttcataattttaatggatattctttggtgtgaataattgaatacaacatttttaaaataaaaaaaaaaaaaaaaaaaaaaaaaaaaaaaaaattttttttttttttttttattccagagattaaagacactagatctttaaccttgaagggcaggcaagaggtcggcaatgctgtcaacatagaagtcagggaccattttcttcttgaacatgcagtcactttcctgattgctcttcacatcctcaaggctccggaattccgggggtgtggtgggctttgatctcaggactctggaggcagaagcaggcagatctctgtgaatatgaggccagcctgcactacacagagctccagaccagtcatggctacatcatgaaaccctgtctcaaaaagaaaataaaaactgttgtgtttctaccatagtgttaaactcagagtctgagtaatgtcgggctgacatgctcgggtgtttaacataccttcagctttgacgaggcgctgaacagtcaaagtctggccttggggagcggtggctgtgtttgtgctcaagtccaccgtgaaatcctgattgtgaatttggacaaccgtgtccttcttcttggccttccatgcaacctccaacttcatgttggtcattttgtcaaaacactgtgtgatgtttttatcaatatactgccattccacatatgtagagatgtagtctgcctggctttccttttctttagccaatcgaatgctcttgatcatgccctcaatctcatctctagcttttatcacgtctctgctaattcctgaaacttgaatcgaagttttcttctggttcatctcaatggtgatgttcagttccttctgaatctcattcagtttctcgtactcctccatgtcaaagtcactgacacactcatcgtcattggtgtaggaaagctgctctttggtaatcagttcctttagccaggagattgttttgttcacactgtctacccctgaaccacatacctggaaaactgtgtgctctattttcttttccaaaaccagggtgttctttttgggggaagcttgcttgggaaagccaagaaaggctaaagagaaaatggaaattaatgtttcttttactcccttcaacatcaaggttaggaatatgtatttcataaaagctaacaactcacaggcaatcttagacatcactgactgcttggcaggcgactgcttggggggagctggagagccttctctttctttcatgttgtcgtaaaaaaattgcagaatatggggctggaagataacaactttaactctcttcacagcctgcactgattttttctggacaaattcttcaatggcatctattatcgcttttgctactacgtttgggtcctgttgagcatttccttcaaaaacaaaaaaagcacatttttaaaaagtcaaggttaagatccacctgcaaaaaaaagctgcaatataagcgaggaattctagttgtcacaggaaataaaaatgtctgttcccactataatcaatgtagactgataatattatgccagcaaatagttttgaagtcctaggcacagtgggaggaggttttgttccacgctgttcataagccaataccccagcaaaagaccttaaaggacaacttgtaatttgggacattcacatctgtcctcttcatctgatctggctcccagtgtcactctctaacacggtccttagagggacaatttatccctgcctctgcttgatcttatgcatgtatctgtattcttccagccatccctggcgacctgatttttctaaggcacccaaaactgtaagctacttcttataatctataattctgagcatattagttagcctgagcctccaggatatctttcttccctatactcagtccagttttagctgcccagaaggattcaaagctgatctacgagtagatcactcctgtctacagcttgttccagatcttgtttctcaagccctggaagccatcagccaggtaagattgtaaaacaatccctttctaatcatgggtgtggcccaaagtgaatggccggaattc

SEQ ID NO: 3 - DNA, 475 bp, mouse
tgtatgggaatagtgtttccatatgatctgttgtctggagtatatgctacatgttcatttactgtacaaaaacccagtgcagctgatgatgcaaagcagtctctctctgtgtacagtgccccacctatttaaaaatcacgtacttgcccagaacactgtgaaacacttaacataagaacaaacgcagcgtctggattctttccaaggagagcagctttctccacaggaacacagtaacaaaagaggtccgccgccatccacacccagccaagacacctcagaggccatagggacaacctccttgctggccaacacctgctggagcaggggcacaggtcccagcaactgatcctcagtggatgggtctgcagccaaagccttaatgggctctcttttgaaggggaaagaaagaatttcaagcttatgatatccaatattattatagttgatgagttagtaaattccaaaaaaaaaa

SEQ ID NO: 4 - DNA, 20 bp, Artificial Sequence
  primer
agggctgtcaatcatgctgg

SEQ ID NO: 5 - DNA, 20 bp, Artificial Sequence
  primer
aaactcacggtcggtgcagc

SEQ ID NO: 6 - DNA, 24 bp, Artificial Sequence
  probe
attaaccctcactaaatgctgtat

SEQ ID NO: 7 - DNA, 30 bp, Artificial Sequence
  probe
cattatgctgagtgatatctttttttttcg

SEQ ID NO: 8 - DNA, 38 bp, Artificial Sequence
  probe
gaacatgtagcatatactccagacaacagatcatatgg

SEQ ID NO: 9 - DNA, 32 bp, Artificial Sequence
  probe
cagcttctccacaggaacacagtaacaaagag

SEQ ID NO: 10 - DNA, 35 bp, Artificial Sequence
  primer
ctatttcacaagagactgaccagccaataaatctc

SEQ ID NO: 11 - DNA, 7581 bp, unknown
  cDNA
cgcccgggcaggtctgttggagggcagttggtcaacctgaccagagagagctgagctggaagaccccactgatggtgtgctgcctttcagtccaggaagaaagaaaggaaggattctgaggatttgggcaaagccacattcctggagaagtctgtatactgatgccaaacccaagagctgagctgctgatgaggcccagggagtagcccacgcgccctgagctgttggctagcaaggccttcctgctccatgtggcatggaaaaattatatggtttgacggatgaaaaggtgaaggcctatctttctctccatccccaggtattagatgaatttgtttctgaaagtgttagtgcagagactgtggaaaagtggctgaagaggaaaaccaacaaagcaaaagatgaaccatctcccaaggaagtcagcaggtaccaggatacgaatatgcagggagtcgtgtacgagctgaacagctacatagagcagcgcctggacacgggcggggacaaccacctgctcctctatgagctcagcagcatcatcaggatagccacaaaagccgacggatttgcactgtacttccttggagagtgcaataatagcctgtgtgtgttcataccacccgggatgaaggaaggccaaccccggctcatccctgcagggcccatcacccagggtaccaccatctctgcctacgtggccaagtctaggaagacgttgttggtagaggatatccttggggatgagcgatttcctcgaggtactggcctggaatcaggaacccgcatccagtctgttctttgcttgcccattgtcactgccattggagacttgattggcatccttgaactgtacaggcactggggcaaagaggccttctgcctcagccatcaggaggttgcaacagccaatcttgcttgggcttccgtagcaatacaccaggtgcaggtgtgtagaggtctcgccaaacagaccgaactgaatgacttcctactcgacgtatcaaagacatactttgataacatagttgccatagactctctacttgaacacatcatgatatatgcaaaaaatctagtgaacgccgaccgctgcgcgctcttccaggtggaccacaagaacaaggagctgtactcggacctgtttgacattggggaggagaaggaggggaagcccatcttcaagaagaccaaggagatcagattttccattgagaaagggattgctggtcaagtggcaagaacaggcgaagtcttgaacattcccgatgcctacgcggaccctcgctttaacagggaggtggacctgtacacaggctacaccacgaggaacattctgtgtatgcccatagtgagccgaggcagcgtgattggcgtggtgcagatggtgaacaagatcagcggtagcgccttctccaagacagacgagaacaacttcaagatgtttgctgtcttctgcgcactggccttgcactgtgctaacatgtaccacaggatccgccactcagaatgcatctacagggttaccatggagaagctttcctaccacagcatctgcacctccgaggagtggcaaggcctcatgcgcttcaacctaccagcacgcatctgccgggacatcgagctattccactttgacattggtcctttcgagaacatgtggcctgggatctttgtctacatgatccatcggtcttgtgggacatcctgttttgaacttgaaaaattgtgccgttttatcatgtctgtgaagaagaactatcggcgggttccttaccacaactggaagcatgcagtcacggtggcacactgcatgtatgccatacttcaaaacaacaatggcctcttcacagacctcgagcgcaaaggcctgctaattgcgtgtctgtgccatgacctggaccacaggggcttcagtaacagctacctgcagaagttcgaccaccccctggcggcgctgtactccacctccaccatggagcaacaccacttctcccagacggtgtccatccttcagctggaagggcacaatatcttctccaccctgagctccagcgagtacgagcaggtgctggagatcatccgcaaagccatcatcgccaccgacctcgccctatactttgggaacaggaagcagttggaggagatgtaccagacagggtcgctgaacctccacaaccagtcccatcgagaccgtgtcatcggcttgatgatgactgcctgtgatctttgctctgtgaccaaactatggccagttacaaaattgacagcgaatgatatatatgcagaattctgggctgagggtgatgagatgaagaagctgggcatacagcccattcctatgatggacagagacaagcgagatgaagtccctcaagggcagctcggattctacaatgctgtggccattccctgctataccaccttgacgcagatcctcccacccacagagcctctgctgaaggcctgcagggataacctcaatcagtgggagaaggtaattcgcggggaagagacagcaatgtggatttcaggcccaggcccggcgcctagcaagagcacacctgagaagctgaacgtgaaggttgaagactgatcctgaagtgacgtcctgatgtctgcccagcaaccgactcaacctgcttctgtgacttcgttctttttgttttcaaggggtgaaaaccccctgtcagaaggtaccgtcgcatatccatgtgaagcagacgactccctgcttgccgcacacacctcggacagtgagcaacccaggctctgccgtgttcagacgtcggctactccgtggctccacctgacctccgaatgctatttgctcccaggccagcactgcactgtctggagggggcagagaccacaggagaggttcttgcctgcatcctcccatgagggtgtggccagttccctagttctgtgccatgctgctgcttggtggcattggttaggaatgggacacacgccccttgttgtgaagtttacatgtgaccttcttataggttaactgagtttgtggcctggacacatgtaatgaaggtcacagtccacaggtgacagagaaatccaaactgttgattacaggtgcactacaggtatgctctttcagtctatctgggggcacataggtgagtctgctccactcagaannaagcatacctctgccctcatccaggggacacagggtacatcccaggcatcggggaactgaagctctcacttcaaaccatgtcaaagaattaaaacacctcccctccccctcactgtagccttcgacaactgcgccaatccctttatacaaagaaaataaaagtaaggcatataaatttcctccagcaagcaaatcttgtgggtaaaaaaaaagcatgtgaatnntaacaacntctanantntcncngnatgttatggcagaattttagtcacgtccaaaacaaaaagattattccagaagatacctcatcctatgcctgaaaggctccacagcatggcgtccgtctcccagggttctgatccgtctcctcacggtgcaatcaggcaggacagagaggagggctgcagggctaccacattgacccagaaggtatctcctctcaccattcagacatccataaggaatgccaaatgctgtattgaatagttctctgtgtgactttctagagaagccaggacaccctgagcctttccnggggaactctaaggagtcacaggttcacaccgtggggattttcaggatagcatggagacagagatccggtcgttgttctcactcgtgagccttgagaaggagagactgaccagaaacactcactcagcactctgcaggagcaggagaagatactttaagatgaatcttggatagattttgatacacccaataccatacacacaggagcttggcatttgcaaagtctattcagtttccttccgcgctctgacccacggttgtagcggagtgggctgaacactgtaacactgtacatgcgatttccccatgggcttctaaaatgtcaccatctcctcccctgctgtgtcctactccatttactggttacaaggtgatgtcaacaagagaagctatcacaacaccagggctgtgcacacgtgcacacacatgtatgcacaagcacacagatgtatgtacagcacacacacacacacacaccccaaaaggagagaaaaggaagaaaacatttataaaaagcgacagctaccccatatcaaaatagtctttcctgtaggaaacaggagctctccataaggaattatcatgagtgtgttctcccatcagtgcactctcccaggggtgctcactgaagctggtccacrtctataaacaggtgacactggctgcagcaaaaagccattcgatccacacaaattgatcttctatcatcttggaatctgaattgcagggaggagcagyatgtaagacgaccgtttaattcaggcattccgaaggcatgagcgcatggattctrtcaccaagcgtataaaaggaccctggcattgggaaacctatgacggactgtttttgctgtagaagtagggattttacagaagtctccttgratttgccctgcctggggcagttttgcagaggaacctgccagagatttattggctggtcagtctcttgtgaaatagtatcatgtgagaaacagtttgtagaaaaaaactatacctgggaagacctttgcaacattgttccttccatgggccaagactcagttaggaggcataaatctgcccggaataaactaggccaggatacagccatgtttagttaataatttggttttagaattcacacaggcaggattggtttttttgtgtcttggcaagtggagcatatttaacatacaggcatgggaatcctgcctcttagcttttcccaccctcttgtctcaccaagttttttctctccaaaggtttccaggaatttctcattaatggctgatgcaaacttagtgaataataatgaatataaacaatgctcacctcaccaaaattatattatttgcagtcatttgtgataacacaaattttatcgcaatggttattatttaatttgtggccacacactgtggttatcttttgttgtggttgtttctgagaaaatgttcttggatatgtaagtgccaataccagtgtgaagtattgatcccgggcagcaaaatacagcctaaggtttgtaaacatcaattctatctcagttcatcagagggcctgagaagctgcggggcagtgtaaagtaaagtatgctgggctggtggtggtcagcctccccttgccaagaagagagcaattgaatcctgtccccagctccctccacgcctgaagagtgaccagtgctggcccgacggatcgctgagatattctcccataatggcaaaaaaataggcagtttgatgtgacctgtttagtgtggctctcctcttttgagcatgtgttagcatttttattttatactcatccagtgaactctgctcttccaagtgtgttcatgtatgtgctagatatattagcacagcctgccttctgctgcacaacgccttagagacccggcctttcaatgagcttagcttgtgctctgtttctgctctcttaggtctaaactatggtgtcagttttaatagaacaaaagtatgcatcttgccttggcttgagccttttcgttttcaatgctgacttctcccctttctctcctgtgctcaccttacctttccagagtgtaagggacaacttttaaggaggcgtgtccctggtaggggcatccctgttcaccaggtgcctgtcatcaccccacttgactgacatctaccctggtgactatgggttcctcttgtttgtagggaacggtggctccaggtggaggcatcaatctgttgggttctggttcccggctgcctttggttttgaaagtctcttctctgtatattcctaccctgcatttgctttgtgtggtgctgatgctgtggcagtaggatcttggatgactctccatcagtcacagactccccctgttgcaaagtgtcaggctgactcgacagtcaccgtaaaatctgagtcagtcacacacaggctgtcagccacggcttccacttgcatggctattctattttcacacgtgagtttctgttgctggctggctgactggcattatctatgctaagttgaaatcaggagtgtgcccagcagagcccatcattctcactgtctttgaaacaaagctgtacggtttgatcgatgaacgtatttaaagcatttcatgcaatgacaaagtgctcagtagtggaaggcaggctgtgaccagtctgcctgctccttactataattgtgaggatttgttactggaacagtacatggaggcctgaccttgtgggggcacagggtggaaccttagctgaatatagtgtgtgtctcaagaggaagtcagggtactagctcagtgctcaatctccaggtactatatatacatttgcccgttttatctctaatgtgaaataaatccccaaacacttgtttatcgtgtagcgtacctaaaagactattctattatgggtgtccccactttcttggtttggtcaccccgatcccccggtcttctgctgtatctagaacagtgactataaatgatgtatgggaatagtgtttccatatgatctgttgtctggagtatatgctacatgttcatttactgtacaaaaacccagtgcagctgatgatgcaaagcagtctctctctgtgtacagtgccccacctatttaaaaatcacgtacaancccagaacactgtgaaacacttaacataagaaacaaacgcagcgtctggattctttccaaggagagcagctttctccacaggaacacagtaacaaaagaggtccgccgccatccacacccagccaagacacctcagaggccatagggacaacctccttgctggccaacacctgctggagcagggcacaggtcccagcaactgatcctcagtggatgggtccgcagtcaaagccttaatgggctctcttttgaaggggaaagaaanntttcaagcttatgatatccaacattattatagttgatgagttagtaaattccgaaaaaaaaagatgattttatatgtatgacataaaaaaaatctttgtaaagtgcgcaagtgcaataatttaaagaggtcttatctttgcatttataaattataaatattgtacatgtgtgtaatttttcatgtattcatttgcagtctttgtatttaaaaaaactttactgttatgtttgtataatagaacattaatcatttattataactcagacaaggtgtaaataaattcataattcaaacagccagtatatatgcatatatgggtgttacattgcaaaaatctctatctttgttctattcacatgcttaaagaagtaagaaatcttttgtggatatgtaattatacatataaagtatatatatatgtatgatacatgaaatatatttagaaatgttcataattttaatggatattctttggtgtgaataattgaatacaacatttttaaaatgaaaaaaaaaaaaaac

SEQ ID NO: 12 - DNA, 7618 bp, mouse
  unsure (7075)...(7076): a or g or c or t/u, unknown or other
cgcccgggcaggtctgttggagggcagttggtcaacctgaccagagagagctgagctggaagaccccactgatggtgtgctgcctttcagtccaggaagaaagaaaggaaggattctgaggatttgggcaaagccacattcctggagaagtctgtatactgatgccaaacccaagagctgagctgctgatgaggcccagggagtagcccacgcgccctgagctgttggctagcaaggccttcctgctccatgtggcatggaaaaattatatggtttgacggatgaaaaggtgaaggcctatctttctctccatccccaggtattagatgaatttgtttctgaaagtgttagtgcagagactgtggaaaagtggctgaagaggaaaaccaacaaagcaaaagatgaaccatctcccaaggaagtcagcaggtaccaggatacgaatatgcagggagtcgtgtacgagctgaacagctacatagagcagcgcctggacacgggcggggacaaccacctgctcctctatgagctcagcagcatcatcaggatagccacaaaagccgacggatttgcactgtacttccttggagagtgcaataatagcctgtgtgtgttcataccacccgggatgaaggaaggccaaccccggctcatccctgcagggcccatcacccagggtaccaccatctctgcctacgtggccaagtctaggaagacgttgttggtagaggatatccttggggatgagcgatttcctcgaggtactggcctggaatcaggaacccgcatccagtctgttctttgcttgcccattgtcactgccattggagacttgattggcatccttgaactgtacaggcactggggcaaagaggccttctgcctcagccatcaggaggttgcaacagccaatcttgcttgggcttccgtagcaatacaccaggtgcaggtgtgtagaggtctcgccaaacagaccgaactgaatgacttcctactcgacgtatcaaagacatactttgataacatagttgccatagactctctacttgaacacatcatgatatatgcaaaaaatctagtgaacgccgaccgctgcgcgctcttccaggtggaccacaagaacaaggagctgtactcggacctgtttgacattggggaggagaaggaggggaagcccatcttcaagaagaccaaggagatcagattttccattgagaaagggattgctggtcaagtggcaagaacaggcgaagtcttgaacattcccgatgcctacgcggaccctcgctttaacagggaggtggacctgtacacaggctacaccacgaggaacattctgtgtatgcccatagtgagccgaggcagcgtgattggcgtggtgcagatggtgaacaagatcagcggtagcgccttctccaagacagacgagaacaacttcaagatgtttgctgtcttctgcgcactggccttgcactgtgctaacatgtaccacaggatccgccactcagaatgcatctacagggttaccatggagaagctttcctaccacagcatctgcacctccgaggagtggcaaggcctcatgcgcttcaacctaccagcacgcatctgccgggacatcgagctattccactttgacattggtcctttcgagaacatgtggcctgggatctttgtctacatgatccatcggtcttgtgggacatcctgttttgaacttgaaaaattgtgccgttttatcatgtctgtgaagaagaactatcggcgggttccttaccacaactggaagcatgcagtcacggtggcacactgcatgtatgccatacttcaaaacaacaatggcctcttcacagacctcgagcgcaaaggcctgctaattgcgtgtctgtgccatgacctggaccacaggggcttcagtaacagctacctgcagaagttcgaccaccccctggcggcgctgtactccacctccaccatggagcaacaccacttctcccagacggtgtccatccttcagctggaagggcacaatatcttctccaccctgagctccagcgagtacgagcaggtgctggagatcatccgcaaagccatcatcgccaccgacctcgccctatactttgggaacaggaagcagttggaggagatgtaccagacagggtcgctgaacctccacaaccagtcccatcgagaccgtgtcatcggcttgatgatgactgcctgtgatctttgctctgtgaccaaactatggccagttacaaaattgacagcgaatgatatatatgcagaattctgggctgagggtgatgagatgaagaagctgggcatacagcccattcctatgatggacagagacaagcgagatgaagtccctcaagggcagctcggattctacaatgctgtggccattccctgctataccaccttgacgcagatcctcccacccacagagcctctgctgaaggcctgcagggataacctcaatcagtgggagaaggtaattcgcggggaagagacagcaatgtggatttcaggcccaggcccggcgcctagcaagagcacacctgagaagctgaacgtgaaggttgaagactgatcctgaagtgacgtcctgatgtctgcccagcaaccgactcaacctgcttctgtgacttcgttctttttgttttcaaggggtgaaaaccccctgtcagaaggtaccgtcgcatatccatgtgaagcagacgactccctgcttgccgcacacacctcggacagtgagcaacccaggctctgccgtgttcagacgtcggctactccgtggctccacctgacctccgaatgctatttgctcccaggccagcactgcactgtctggagggggcagagaccacaggagaggttcttgcctgcatcctcccatgagggtgtggccagttccctagttctgtgccatgctgctgcttggtggcattggttaggaatgggacacacgccccttgttgtgaagtttacatgtgaccttcttataggttaactgagtttgtggcctgggacacatgtaatgaaggtcacagtccacaggtgacagagaaatccaaactgttgattacaggtgcactacaggtatgctctttcagtctatctgggggcacataggtgagtctgctccactcagaaggaagcatacctctsccctcatccaggggacacagggtacatcccaggcatcggggaactgaagctctcacttcaaaccatgtcaaagaattaaaacacctcccctccccctcactgtagccttcggcaactgcgccaatccctttatacaaagaaaatataagtaaggcatataaatttcctccagcaagcaaatcttgtgggtaaaaaaaaaaaatgtgaattttaacaacctctatattttcactgtatgttatggcagaattttagtcacgtccaaaacaaaagattattccagaagatacctcatcctatgcctgaaagctccacagcatggcgtccgtctcccagggttctgatccgtctcctcacggtgcaatcaggcaggacaggaggaggtgcagggctaccacattgacccagatggtatctcctctcaccattcagacatccataaggaatgccaaatgctgtattgaatagttctcctgtgtgactttctagagaagccaggacacccctgagcctttcctgggaactcctaaggaagtcacaggttcacaccgtggggattttcaggatagcatggagaccagagaatcccggttcggttgttctcactcggtgagccttgagaaggaagagactgaccagaaacactcactcagcactctggcaggagcaggagaagatactttaagatgaatctttgggatagattttgatacacccaataccatacacacaggagcttggcatttgcaaagtctattcagtttccttccacactctgacccacggttgtagcggagtgggctgaacactgtaacactgtacatgcgatttccccatgggcttctaaaatgtcaccatctcctcccctgctgtgtcctactccatttactggttacaaggtgatgtcaacaagagaagctatcacaacaccagggctgtgcacacgtgcacacacatgtatgcacaagcacacagatgtatgtacagcacacacacacacacacaccccaaaaggagagaaaaggaagaaaacatttataaaaagcgacagctacccccatattcaaaaatagttcttttccctgtagggaaacaggtagctctccataaggaaattatcatgagtgtgttctcccatcagtgcacttctcccaggggtgctcactgaagctggtccacgtctataaacaggtgacactggctgcagcaaaaagccattcgatccacacaaattgatcttctatcatcttggaatctgaattgcagggaggagcagcatgtaagacgaccgtttaattcaggcattccgaaggcatgagcgcatggattctgtcaccaagcgtataaaaggaccctggcattgggaaacctatgacggactgtttttgctgtagaagtagggattttacagaagtctccttggatttgccctgcctggggcagttttgcagaggaacctgccagagatttattggctggtcagtctcttgtgaaatagtatcatgtgagaaacagtttgtagaaaaaaactatacctgggaagacctttgcaacattgttccttccatgggccaagactcagttaggaggcataaatctgcccggaataaactaggccaggatacagccatgtttagttaataatttggttttagaattcacacaggcaggattggtttttttgtgtcttggcaagtggagcatatttaacatacaggcatgggaatcctgcctcttagcttttcccaccctcttgtctcaccaagttttttctctccaaaggtttccaggaatttctcattaatggctgatgcaaacttagtgaataataatgaatataaacaatgctcacctcaccaaaattatattatttgcagtcatttgtgataacacaaattttatcgcaatggttattatttaatttgtggccacacactgtggttatcttttgttgtggttgtttctgagaaaatgttcttggatatgtaagtgccaataccagtgtgaagtattgatcccgggcagcaaaatacagcctaaggtttgtaaacatcaattctatctcagttcatcagagggcctgagaagctgcggggcagtgtaaagtaaagtatgctgggctggtggtggtcagcctccccttgccaagaagagagcaattgaatcctgtccccagctccctccacgcctgaagagtgaccagtgctggcccgacggatcgctgagatattctcccataatggcaaaaaaataggcagtttgatgtgacctgtttagtgtggctctcctcttttgagcatgtgttagcatttttattttatactcatccagtgaactctgctcttccaagtgtgttcatgtatgtgctagatatattagcacagcctgccttctgctgcacaacgccttagagacccggcctttcaatgagcttagcttgtgctctgtttctgctctcttaggtctaaactatggtgtcagttttaatagaacaaaagtatgcatcttgccttggcttgagccttttcgttttcaatgctgacttctcccctttctctcctgtgctcaccttacctttccagagtgtaagggacaacttttaaggaggcgtgtccctggtaggggcatccctgttcaccaggtgcctgtcatcaccccacttgactgacatctaccctggtgactatgggttcctcttgtttgtagggaacggtggctccaggtggaggcatcaatctgttgggttctggttcccggctgcctttggttttgaaagtctcttctctgtatattcctaccctgcatttgctttgtgtggtgctgatgctgtggcagtaggatcttggatgactctccatcagtcacagactccccctgttgcaaagtgtcaggctgactcgacagtcaccgtaaaatctgagtcagtcacacacaggctgtcagccacggcttccacttgcatggctattctattttcacacgtgagtttctgttgctggctggctgactggcattatctatgctaagttgaaatcaggagtgtgcccagcagagcccatcattctcactgtctttgaaacaaagctgtacggtttgatcgatgaacgtatttaaagcatttcatgcaatgacaaagtgctcagtagtggaaggcaggctgtgaccagtctgcctgctccttactataattgtgaggatttgttactggaacagtacatggaggcctgaccttgtgggggcacagggtggaaccttagctgaatatagtgtgtgtctcaagaggaagtcagggtactagctcagtgctcaatctccaggtactatatatacatttgcccgttttatctctaatgtgaaataaatccccaaacacttgtttatcgtgtagcgtacctaaaagactattctattatgggtgtccccactttcttggtttggtcaccccgatcccccggtcttctgctgtatctagaacagtgactataaatgatgtatgggaatagtgtttccatatgatctgttgtctggagtatatgctacatgttcatttactgtacaaaaacccagtgcagctgatgatgcaaagcagtctctctctgtgtacagtgccccacctatttaaaaatcacgtacaancccagaacactgtgaaacacttaacataagaaacaaacgcagcgtctggattctttccaaggagagcagctttctccacaggaacacagtaacaaaagaggtccgccgccatccacacccagccaagacacctcagaggccatagggacaacctccttgctggccaacacctgctggagcagggcacaggtcccagcaactgatcctcagtggatgggtccgcagtcaaagccttaatgggctctcttttgaaggggaaagaaanntttcaagcttatgatatccaacattattatagttgatgagttagtaaattccgaaaaaaaaagatgattttatatgtatgacataaaaaaaatctttgtaaagtgcgcaagtgcaataatttaaagaggtcttatctttgcatttataaattataaatattgtacatgtgtgtaatttttcatgtattcatttgcagtctttgtatttaaaaaaactttactgttatgtttgtataatagaacattaatcatttattataactcagacaaggtgtaaataaattcataattcaaacagccagtatatatgcatatatgggtgttacattgcaaaaatctctatctttgttctattcacatgcttaaagaagtaagaaatcttttgtggatatgtaattatacatataaagtatatatatatgtatgatacatgaaatatatttagaaatgttcataattttaatggatattctttggtgtgaataattgaatacaacatttttaaaatgaaaaaaaaaaaaaaaaaaaaaaaaaaaaaaaaa